(19) United States
(12) Patent Application Publication
(10) Pub. No.: US 2006/0035246 A1
(43) Pub. Date: Feb. 16, 2006

(54) Chromogenic in situ hybridization methods, kits, and compositions

(76) Inventors: Rina Wu, Brisbane, CA (US); Abbey Marquez, Daly City, CA (US)

Correspondence Address: CASTELLANO MALM FERRARIO & BUCK PLLC, 2121 K STREET, NW, SUITE 800, WASHINGTON, DC, 20037, US

(21) Appl. No.: 11/114,798

(22) Filed: Apr. 26, 2005

Related U.S. Application Data
(63) Continuation-in-part of application No. 10/173,525, filed on Jun. 17, 2002.
Application No. 10/173,525 is a continuation-in-part of application No. 09/952,851, filed on Sep. 14, 2001, now Pat. No. 6,942,970.

(51) Int. Cl. C12Q 1/68; C40B 40/08

(52) U.S. Cl. 435/6

(57) ABSTRACT

The present invention relates to chromogenic (calorimetric) in situ hybridization (CISH) and nucleic acid probes useful for in situ hybridization. Specifically, the present invention provides methods, kits, and compositions for performing bright field cancer diagnostics employing chromogenic in situ hybridization (e.g. to detect gene amplifications, gene translocations, and chromosome polysomy).

The present application is a continuation-in-part of U.S. application Ser. No. 10/173,525, filed Jun. 17, 2002, which is a continuation-in-part of U.S. application Ser. No. 09/952,851, filed Sep. 14, 2001, both of which are herein incorporated by reference.

Filed herewith as Table 15 and expressly incorporated by reference is an appendix contained on one compact disc, submitted as an identical set labeled “Copy 1” and “Copy 2,” for a total of two compact discs. Each compact disc was prepared in IBM-PC format and is compatible with the MS-Windows operating system. The compact disc contains the following thirteen files, in ASCII format:

| | | Creation | Size | |
|---|---|---|---|---|
| | File Name | Date | (bytes) | SEQ ID NO |
| 1. | CTD-2307E2.txt | Apr. 25, 2005 | 89,420 | SEQ ID NO: 46 |
| 2. | CTD-2504F18.txt | Apr. 25, 2005 | 205,131 | SEQ ID NO: 47 |
| 3. | CTD-2270N9.txt | Apr. 25, 2005 | 179,668 | SEQ ID NO: 48 |
| 4. | CTD-3170A23.txt | Apr. 25, 2005 | 126,606 | SEQ ID NO: 49 |
| 5. | CTD-2021M22.txt | Apr. 25, 2005 | 95,889 | SEQ ID NO: 50 |
| 6. | RP11-375I18.txt | Apr. 25, 2005 | 209,365 | SEQ ID NO: 51 |
| 7. | CTD-3051O18.txt | Apr. 25, 2005 | 172,549 | SEQ ID NO: 52 |
| 8. | CTD-2556F3.txt | Apr. 25, 2005 | 204,586 | SEQ ID NO: 53 |
| 9. | RP11-715E11.txt | Apr. 25, 2005 | 206,937 | SEQ ID NO: 54 |
| 10. | CTD-2559C16.txt | Apr. 25, 2005 | 180,046 | SEQ ID NO: 55 |
| 11. | RP11-718I15.txt | Apr. 25, 2005 | 191,058 | SEQ ID NO: 56 |
| 12. | CTD-2571L23.txt | Apr. 25, 2005 | 185,691 | SEQ ID NO: 57 |
| 13. | bk256d12.txt | Apr. 25, 2005 | 327,588 | SEQ ID NO: 58 |

### FIELD OF THE INVENTION

The present invention relates to chromogenic (colorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, deletion, and chromosome aneuploidy).

### BACKGROUND OF THE INVENTION

Characterization chromosome aberrations have been studied in a wide range of tumors. Specific oncogene and tumor suppressor gene targets affected by these chromosomal abnormalities have been characterized in many tumors. One such target is the HER2 gene. HER2 gene amplification or HER2 protein overexpression has been identified in 10-34% of invasive breast cancers according to a series of 52 published studies including more than 16,000 patients and using different methodologies (See, Ross et al., Am. J. Clin. Pathol., 1999; 112:S53-67, herein incorporated by reference).

Identification of HER2 status is important for determining the prognosis of patients who have invasive breast cancer, as well as for selecting a subgroup with metastasis HER2 overexpression for therapy with trastuzumab (HERCEPTIN), a humanized anti-HER2 monoclonal antibody (See, Shak et al., Cancer Res. 199; 6:71-7; and Cobleigh et al., J. Clin. Oncol., 1999; 17:2639-48, both of which are herein incorporated by reference). HERCEPTIN has been found to be effective only in patients whose tumors show HER2 gene amplification and/or HER protein overexpression. As such, accurate, consistent, and straightforward methods for evaluation of HER2 status have become increasingly important.

Immunohistochemical (IHC) staining has been the predominant method of determining HER2 status in breast cancer specimens. It is relatively easy to perform and has a rapid turnaround time, and a relatively low cost (See, Ross et al. above, and Hanna et al., Mod. Pathol., 1999, 12:827-34, herein incorporated by reference). However, many commercially available antibodies have demonstrated wide variation in sensitivity and specificity for FFPE (formalin fixed paraffin embedded) tissue samples, and the effect of the tissue fixative and pretreament have a substantial effect on HER2 IHC staining (See, Ross et al. above; Jacobs et al., J. Clin. Oncol. 1999, 17:1974-1987; Espinoza et al., J. Clin. Oncol. 1999, 17:2293B; and Penault-Llorca et al., J. Pathol. 1994, 173:65-75, all of which are herein incorporated by reference). In addition, the lack of a universal scoring system and interobserver differences in interpretation of HER2 IHC results is also source of unwanted variation.

Overexpresion of the HER2 protein generally (>95%) results from HER2 gene amplification (See, Slamon et al., Science, 1989; 244:707-12, herein incorporated by reference). Fluorescence in situ hybridization (FISH) is believed by many to be the most sensitive technique for quantitative evaluation of HER2 gene status in breast cancer cells and also believed to be a valid alternative to IHC in FFPE tissue sections (See, Pauletti et al., J. Clin. Oncology, 2000, 18:3651-64, herein incorporated by reference.). Patients who were positive by FISH but negative by IHC had a worse survival rate than those who had HER2 overexpression but an absence of gene amplification (See, Pauletti et al., above). Therefore, HER2 amplification could provide more meaningful prognostic information than HER2 overexpression in breast cancer patients. In addition, FISH quantifies the number of gene copies in the cancer cell, which objectively reflects the HER2 gene status of tumors, whereas IHC is a more subjective test. Therefore, FISH can be easier to interpret than IHC. However, FISH methodology also has many disadvantages.

Evaluation of FISH requires a modern and expensive fluorescence microscope equipped with high-quality 60× or 100× oil immersion objectives and multi-band-pass fluorescence filters, which is not used in most routine diagnostic laboratories. The fluorescence signals can fade within several weeks, and the hybridization results are typically recorded with an expensive CCD camera. Therefore, analysis and recording of FISH data is expensive and time consuming. Most importantly, tissue section morphology is not optimal in FISH on FFPE, a particular problem for distinguishing invasive breast cancer and breast carcinoma in situ, where HER2 gene amplification or protein overexpression may have different clinical significance. All of these limitations make FFPE FISH cumbersome for routine work (See, Jacobs et al. above, and Tanner et al., Am. J. Pathol. 2000, 157:1467-72, herein incorporated by reference).

Therefore, what is needed are methods, kits and compositions that accurately identify cancer marker gene status, such as HER2 gene status, that do not require expensive fluorescence detection equipment, allow cell morphology and ISH signal to be viewed at the same time, and provide accurate results using standard equipment, such as bright field-microscopes.

### SUMMARY OF THE INVENTION

The present invention relates to chromogenic (calorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, and chromosome polysomy). In preferred embodiments, the present invention provides CISH methods, kits and compositions for detecting HER2 gene status.

In some embodiments, the present invention provides methods for performing chromogenic in-situ hybridization, comprising; a) providing; i) a biological sample (e.g. tumor biopsy), ii) a labeled subtracted probe library, wherein the subtracted probe library is configured to hybridize to a target region, iii) pretreatment buffer, iv) enzyme digestion solution, v) a colorimetric substrate, and vi) a detection molecule conjugated to a colorimetric substrate enzyme; b) preheating the biological sample in the pretreatment buffer at a temperature of at least 96 degrees Celsius, c) exposing the biological sample to the enzyme digestion solution, d) contacting the biological sample with the subtracted probe library under conditions such that the subtracted probe library hybridizes to the target region, e) adding the detection molecule to the biological sample under conditions such that the detection molecule binds; i) to the labeled subtracted probe library, or ii) an intermediate molecule linked to the subtracted probe library, f) adding the colorimetric substrate to the biological sample under conditions such that the subtracted probe library is detected.

In particular embodiments, the present invention provides methods for performing chromogenic in-situ hybridization, comprising; a) preheating a biological sample (e.g. tumor biopsy) in a pretreatment buffer at a temperature of at least 96 degrees Celsius, b) exposing the biological sample to a enzyme digestion solution, c) contacting the biological sample with a subtracted probe library under conditions such that the subtracted probe library hybridizes to a target region in the biological sample, d) adding a detection molecule linked to an enzyme to the biological sample under conditions such that the detection molecule binds; i) to the labeled subtracted probe library, or ii) an intermediate molecule linked to the subtracted probe library, and e) adding a colorimetric substrate to the biological sample. In other embodiments, the method further comprises step f) detecting the presence or absence of the target region in the biological sample. In additional embodiments, the detecting comprising visualizing the colorimetric substrate with a microscope (e.g. bright-field microscope).

In some embodiments, the subtracted probe library is configured for detecting HER2 gene amplification. In particular embodiments, the target region comprises the HER2 gene. In other embodiments, the subtracted probe library is configured for detecting topoIIα gene amplification. In certain embodiments, the target region comprises the topoIIα gene (e.g. and does not encompass the HER2 gene sequence). In some embodiments, the subtracted probe library is configured for detecting EGFR (epidermal growth factor receptor) gene amplification. In particular embodiments, the target region comprises the EGFR gene. In other embodiments, the subtracted probe library is configured for detecting N-MYC gene amplification. In additional embodiments, the target region comprises the N-MYC gene.

In particular embodiments, the subtracted probe library is configured for detecting a chromosome 19q deletion (e.g. deletion between 19q13.3 and 19q13.4). In certain embodiments, a subtracted probe library generated from clone CDT-2571L23 (see Table 15) or a clone substantially similar thereto (e.g. able to hybridize to said clone) is employed for such detection. In other embodiments, the subtracted probe library is configured for detecting a chromosome 18q deletion (e.g. a deletion of the long arm of chromosome 18). In certain embodiments, the subtracted probe library is configured for detecting a deletion of chromosome 18q21. In some embodiments, a subtracted probe library generated from clones RP11-375I18, CTD-3051O18, and RP11-718I15 (see Table 15) or a collection of clones substantially similar thereto (e.g. able to hybridize to said clones) is employed for such detection. In additional embodiments, the subtracted probe library is configured for detecting a chromosome 1p deletion (e.g. a deletion in 1p36). In some embodiments, a subtracted probe library generated from clones CTD-2307E2, CTD-2504F18, and CTD-2270N9 (see Table 15) or a collection of clones substantially similar thereto (e.g. able to hybridize to said clones) is employed for such detection. In additional embodiments, the subtracted probe library is configured for detecting a deletion of the NF2 gene and/or a chromosome 22q deletion. In some embodiments, a subtracted probe library generated from clones bk256d12, CTD-3170A23, and CTD-2021M22 (see Table 15) or a collection of clones substantially similar thereto (e.g. able to hybridize to said clones) is employed for such detection.

In some embodiments, the subtracted probe library comprises a probe pair library. In other embodiments, the probe pair comprises a split-apart probe pair. In particular embodiments, the probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene. In other embodiments, the probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene. In further embodiments, the probe pair comprises; i) a first subtracted probe library configured to hybridize to a first region of chromosome 18 that is centromeric with respect to the MALT1 gene, and ii) a second subtracted probe library configured to hybridize to a second region of chromosome 18 that is teleomeric with respect to the MALT1 gene. In some embodiments, the first subtracted probe library is generated from clones RP11-375I18, CTD-305O18, and RP11-718I15 (see Table 15) or a collection of clones substantially similar thereto (e.g. able to hybridize to said clones), and the second subtracted probe library is generated from clones CTD-2556F3, RP11-715E11, and CTD-2559C16 (see Table 15) or a collection of clones substantially similar thereto (e.g. able to hybridize to said clones). In certain embodiments, the probe pair comprises; i) a first probe library configured to detect a chromosome 19q deletion; and ii) a second probe library configured to detect a chromosome 1p deletion.

In certain embodiments, the preheat temperature is at least 98 degrees Celsius (e.g. 98, 99 or 100 degrees Celsius). In other embodiments, the preheat temperature is from 96 degrees Celsius to 100 degrees Celsius (e.g. 98-100 degrees Celsius). In some embodiments, the preheating is accomplished with a pressure cooker, a hot plate, or a microwave oven. In other embodiments, the biological sample, during the preheating step, is inside an enclosed container.

In some embodiments, the enzyme digestion solution comprises pepsin (e.g., a solution having about 0.0625% pepsin, pH 2.3, or a solution having about 0.075% pepsin, pH 2.0). For example, in some embodiments, the enzyme digestion solution comprises between 0.060% pepsin and 0.080% pepsin. In other embodiments, the enzyme digestion solution comprises between 0.070% pepsin and 0.080% pepsin. In other embodiments, the pretreatment buffer comprises TRIS-EDTA (e.g. 0.1 M Tris/0.05 EDTA, pH 7.0). In other embodiments, the pretreament buffer is TRIS.

In certain embodiments, the detection molecule is avidin, streptavidin or biotin. In some embodiments, the detection molecule is an antibody. In particular embodiments, the detection molecule is linked to a plurality of enzymes via a polymer. In additional embodiments, the intermediate molecule is a primary antibody, and the detection molecule is a secondary antibody that binds to the primary antibody.

In some embodiments, the enzyme comprises a peroxidase (e.g. a horseradish peroxidase). In other embodiments, the enzyme is HRP or AP. In other embodiments, the method further comprises performing immunohistochemistry on the biological sample with antibodies specific for proteins expressed by the target region. In some embodiments, the subtracted probe library comprises digoxigenin, FITC, avidin, streptavidin, or biotin. In additional embodiments, the colorimetric substrate comprises diaminobenzidine or FAST RED.

In certain embodiments, the subtracted probe library comprises a heterogeneous mixture of labeled nucleic acid probes about 0.1 kb to about 8 kb in length (e.g. about 0.5 to about 4 kb in length, or about 0.2 to 0.5 kb in length). In some embodiments, the target region is about 50 kb to about 500 kb, or 1.5 to 5.0 megabases in length. In other embodiments, the target region is associated with human cancer gene aberrations. In certain embodiments, the biological sample is a tumor sample (e.g. a breast cancer biopsy tissue sample). In some embodiments, the biological sample is fixed on a surface (e.g. microscope slide).

In some embodiments, the subtracted probe library is about 90 percent free of repeat sequences. In other embodiments, the subtracted probe library is about 95 percent free of repeat sequences. In certain embodiments, the biological sample is a paraffin-embedded tissue sample (e.g. formalin-fixed paraffin-embedded tissue sample).

In particular embodiments, the preset invention provides kits for performing chromogenic in-situ hybridization, comprising; a) a labeled subtracted probe library, wherein the subtracted probe library is configured to hybridize to a target region, b) a written insert component, wherein the written inert component comprises instructions for performing chromogenic in-situ hybridization. In other embodiments, the kit further comprises at least one of the following; a pretreatment buffer, an enzyme digestion solution, a colorimetric substrate, and a detection molecule conjugated to a calorimetric substrate enzyme.

In additional embodiments, the instructions for performing chromogenic in-situ hybridization comprises instructions for visualizing the calorimetric substrate with a bright-field microscope. In certain embodiments, the subtracted probe library is configured for detecting HER2 gene amplification, topoIIα gene amplification, EGFR gene amplification, N-MYC gene amplification, chromosome 19q deletion, chromosome 18q deletion, chromosome 22q deletion, or chromosome 1p deletion.

In some embodiments, the subtracted probe library comprises a probe pair library. In other embodiments, the probe pair comprises a split-apart probe pair. In particular embodiments, the probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene. In other embodiments, the probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene. In further embodiments, the probe pair comprises; i) a first probe library configured to hybridize to a first region of chromosome 18 that is centromeric with respect to the MALT1 gene, and ii) a second probe library configured to hybridize to a second region of chromosome 18 that is teleomeric with respect to the MALT1 gene. In certain embodiments, the probe pair comprises; i) a first probe library configured to detect a chromosome 19q deletion; and ii) a second probe library configured to detect a chromosome 1p deletion.

In other embodiments, the written insert component comprises instructions for preheating a biological sample in a pretreament buffer to a temperature of at least 96 degrees Celsius. In some embodiments, the written insert component comprises instructions for preheating a biological sample in a pretreament buffer to a temperature of at least 98 degrees Celsius (e.g. 98-100 degrees Celsius). In certain embodiments, the instructions for preheating indicate that the temperature is accomplished with a pressure cooker, a hot plate or a microwave oven. In particular embodiments, the instructions for preheating further indicate that the biological sample, during the preheating step, should be inside an enclosed container.

In some embodiments, the present invention provides methods for diagnosing and treating a subject, comprising; a) preheating a biological sample from a subject in a pretreatment buffer, b) exposing the biological sample to a enzyme digestion solution, c) contacting the biological sample with a subtracted probe library under conditions such that the subtracted probe library hybridizes to a target region in the biological sample, wherein the target region comprises the HER2 gene sequence, d) adding a detection molecule linked to an enzyme to the biological sample under conditions such that the detection molecule binds; i) to the labeled subtracted probe library, or ii) an intermediate molecule linked to the subtracted probe library, e) adding a calorimetric substrate to the biological sample, f) detecting the target region by visualizing the colorimetric substrate with a bright-field microscope, thereby determining that the biological sample has amplification of the HER2 gene sequence, and g) identifying the subject as suitable for treatment with anti-HER2 antibodies. In particular embodiments, the method further comprises step h) administering the anti-HER2 antibodies (e.g. HERCEPTIN) to the subject.

In some embodiments, the present invention provides methods for identifying suitable treatment for a subject, comprising: screening a biological sample for the presence or absence of gene amplification in both HER-2/neu and topoIIα, wherein the biological sample is suspected of containing breast cancer cells and is obtained from the subject.

In other embodiments, the present invention provides methods for identifying suitable treatment for a subject, comprising: a) screening a biological sample for the presence or absence of: i) gene amplification in topoIIα and ii) gene amplification in HER-2/neu or overexpression of HER2, wherein the biological sample is suspected of containing breast cancer cells and is obtained from the subject, and b) identifying the subject as suitable for; i) anti-HER2 antibody-free anthracycline treatment, or ii) anthracycline-free anti-HER2 antibody treatment.

In some embodiments, the identifying the subject as suitable for anti-HER2 antibody-free anthracycline treatment comprises determining the presence of gene amplification in both said HER-2/neu and said topoIIα, or determining the presence of gene amplification in said topoIIα gene and overexpression of HER2. In other embodiments, the identifying the subject as suitable for anthracyline-free anti-HER2 antibody treatment comprises determining: i) the presence of gene amplification in the HER-2/neu or overexpression of HER2, and ii) the absence of gene amplification in the topoIIα.

In certain embodiments, the determining comprises performing in-situ hybridization methods on the biological sample with HER-2/neu and topoIIα specific probes. In additional embodiments, the in-situ hybridization methods comprise fluorescent in situ hybridization and/or chromogenic in situ hybridization. In other embodiments, the determining comprises performing in situ hybridization on the biological sample with a topoIIα specific probe, and performing immunohistochemical methods on the biological sample with anti-HER2 antibodies. In some embodiments, the methods further comprise step c) administering an anthracycline to the subject without administering anti-HER2 antibodies.

In certain embodiments, the identifying the subject as suitable for anthracyline-free anti-HER2 antibody treatment comprises determining: i) the presence of gene amplification in the HER-2/neu or overexpression of HER2, and ii) the absence of gene amplification in the topoIIα. In certain embodiments, the determining comprises performing in situ hybridization methods on the biological sample with HER-2/neu and topoIIα specific probes. In additional embodiments, the in situ hybridization methods comprise fluorescent in-situ hybridization and/or chromogenic in situ hybridization. In other embodiments, the determining comprises performing in situ hybridization on the biological sample with a topoIIα specific probe, and performing immunohistochemical methods on the biological sample with anti-HER2 antibodies. In particular embodiments, the methods further comprise step c) administering anti-HER2 antibodies (e.g. HERCEPTIN) to the subject without administering an anthracycline.

In some embodiments, the present invention provides kits for identifying suitable treatment for a subject, comprising: a) reagents for screening a biological sample from a subject, suspected of containing breast cancer cells, for the presence or absence of; i) gene amplification in topoIIα, and ii) gene amplification in HER-2/neu or HER overexpression, and b) a written insert component, wherein the written insert component comprises instructions for employing the reagents for identifying the subject as suitable for; i) anti-HER2 antibody-free anthracycline treatment, or ii) anthracycline-free anti-HER2 antibody treatment. In particular embodiments, the instructions for identifying the subject as suitable for anti-HER2 antibody-free anthracycline treatment comprises instructions for determining the presence of gene amplification in both the HER-2/neu and the topoIIα, or determining the presence of topoIIα gene amplification and HER-2/neu amplification or HER2 overexpression, employing the reagents.

In additional embodiments, the instructions for determining comprises instructions for performing in-situ hybridization methods (e.g., FISH and/or CISH) on the biological sample with HER-2/neu and topoIIα specific probes. In some embodiments, the instructions for determining comprises instructions for performing in-situ hybridization on the biological sample with a topoIIα specific probe, and instructions for performing immunohistochemical methods on the biological sample with anti-HER2 antibodies.

In other embodiments, the reagents comprise at least one of the following: a labeled subtracted probe library, wherein the subtracted probe library is configured to hybridize to a HER-2/neu or topoIIα, pretreatment buffer, an enzyme digestion solution, a colorimetric substrate, and a detection molecule conjugated to a colorimetric substrate enzyme. In some embodiments, the instructions for identifying the subject as suitable for anthracyline-free anti-HER2 antibody treatment comprises instructions for determining: i) the presence of gene amplification in the HER-2/neu or HER2 overexpression, and ii) the absence of gene amplification in the topoIIα. In certain embodiments, the instructions for determining comprises instructions for performing in-situ hybridization methods (e.g. FISH and/or CISH) on the biological sample with HER-2/neu and topoIIα specific probes.

In other embodiments, the instructions for determining comprises instructions for performing in-situ hybridization on the biological sample with a topoIIα specific probe, and instructions for performing immunohistochemical methods on the biological sample with anti-HER2 antibodies.

The present invention provides methods for diagnosing and treating cancer, and in particular methods for determining the susceptibility of subjects suspected of having breast cancer to treatment with topoisomerase II inhibitors. The present invention also provides in situ hybridization probes and kits for specifically detecting topoIIα gene sequences.

In some embodiments, the present invention provides methods for identifying a candidate for topoisomerase II inhibitor treatment, comprising: a) providing a candidate subject suspected of having cancer cells; b) detecting a copy number for both HER-2/neu and topoIIα in the cancer cells; and c) identifying the candidate subject as being suitable for treatment with a topoisomerase II inhibitor, wherein the identifying comprises demonstrating amplification of the copy number for both HER-2/neu and topoIIα. In some embodiments, the candidate subject has cancer cells. In other embodiments, the candidate subject has been previously diagnosed as having cancer cells from diseases including, but not limited to, leukemia, brain cancer, kidney cancer, lymphoma, eye cancer, connective tissue cancer, Hodgkin's disease, bone cancer, testicular cancer, cervical cancer, thyroid cancer, melanoma, skin cancer, uterine cancer, lung cancer, colon cancer, rectal cancer, ovarian cancer, bladder cancer, larynx cancer, prostate cancer, stomach cancer, breast cancer, and pancreatic cancer. In preferred embodiments, the candidate subject has breast cancer cells. In particularly preferred embodiments, the candidate subject has metastatic breast cancer cells.

The present invention provides methods for identifying candidates for topoisomerase II inhibitor treatment, comprising: a) providing a candidate subject suspected of having breast cancer cells; b) detecting a copy number for both HER-2/neu and topoIIα in the breast cancer cells; and c) identifying the candidate subject as suitable for treatment with a topoisomerase II inhibitor, wherein the identifying comprises demonstrating amplification of the copy number for both HER-2/neu and topoIIα. In certain embodiments, the demonstrating comprises comparing the copy number of both HER-2/neu and topoIIα to a control copy number. In further embodiments, the copy number of HER-2/neu is at least 1.5 times greater than the control copy number. In additional embodiments, the copy number of topoIIα is at least 1.5 times greater than the control copy number. In further embodiments, the method further comprises step d) treating the candidate subject with a topoisomerase II inhibitor.

In some particularly preferred embodiments, the candidate subject is a human. In other embodiments, the candidate subject is a non-human animal. In some embodiments, the animal is a mammal (e.g., human, cat, dog, pig, or cow). In some preferred embodiments, the animal is a female, in other embodiments, the animal is a male. In some embodiments, the candidate subject has breast cancer cells (e.g., previously diagnosed as having breast cancer cells). In some preferred embodiments, the breast cancer cells are metastatic.

In some embodiments of the present invention, the detecting step comprises obtaining a tissue sample (e.g., biopsy) comprising the breast cancer cells from the candidate subject. In further embodiments, the detecting step further comprises contacting the tissue sample comprising the breast cancer cells with a first probe specific for the HER-2/neu and a second probe specific for the topoIIα. In certain embodiments, the second probe comprises at least about 100,000 nucleotides (e.g. a probe library comprising 100,000 nucleotides) and hybridizes to a target region of human chromosome seventeen under in situ hybridization conditions, and wherein the target region contains topoIIα gene sequence, but does not contain HER-2/neu gene sequence.

In other embodiments, the first and second probes are detectably labeled nucleic acid. In further embodiments, the first probe is nucleic acid capable of hybridizing to HER-2/neu. In additional embodiments, the second probe is nucleic acid capable of hybridizing to topoIIα. In further embodiments, the first and second probes are detectably labeled. In particular embodiments, the detecting step comprises fluorescent in situ hybridization. In some embodiments, the detecting step comprises Southern blotting (hybridization) or Northern blotting (hybridization). In additional embodiments, the detecting step comprises Western blotting. In further embodiments, the detecting step comprises enzyme immunoassay (EIA). In certain embodiments, the detecting step comprises enzyme-linked immunosorbent assay (ELISA). In certain embodiments, the first and/or second probe is labeled with digoxigenin, and the first and/or second probe is fluorescently labeled. In other embodiments, the first and/or second probe is detected by chromogenic in situ hybridization. In certain embodiments, the first and/or second probe is detected by fluorescent in situ hybridization. In further embodiments, the detecting step comprises contacting the tissue sample comprising the breast cancer cells with an antibody specific for HER2 (e.g., in order to detect a copy number for HER-2/neu) and a nucleic acid probe specific for topoIIα. In some particularly preferred embodiments, the detecting step comprises immunohistochemical detection and fluorescent in situ hybridization (FISH). However, it should be noted that any suitable method for detection of topoIIα and HER-2/neu finds use with the present invention.

The present invention further provides methods for identifying candidates for topoisomerase II inhibitor treatment, comprising: a) providing a candidate subject suspected of having breast cancer cells; b) detecting a copy number for both HER-2/neu and topoIIα in the breast cancer cells, wherein the detecting comprises contacting the breast cancer cells with a first probe specific for HER-2/neu, a second probe specific for topoIIα (e.g. a topoIIα probe library comprising fragments), and a control probe; and c) identifying the candidate subject as being suitable for treatment with a topoisomerase II inhibitor, wherein the identifying comprises demonstrating amplification of the copy number for both HER-2/neu and the topoIIα. In particular embodiments, the control probe is specific for human chromosome 17. In some particularly preferred embodiments, the topoisomerase II inhibitor is an anthracycline. In other embodiments, the anthracycline is selected from doxorubicin and epirubicin. In further embodiments, the breast cancer cells are metastatic.

The present invention provides methods for identifying candidates for topoisomerase II inhibitor treatment, comprising: a) providing a candidate subject comprising breast cancer cells, wherein the breast cancer cells comprise an amplified copy number for HER-2/neu, b) detecting a copy number topoIIα in the breast cancer cells; and c) identifying the candidate subject as suitable for treatment with a topoisomerase II inhibitor, wherein the identifying comprises demonstrating amplification of the copy number for topoIIα. In particular embodiments, the demonstrating comprises comparing the copy number for topoIIα to a control copy number. In further embodiments, the copy number of the topoIIα is at least 1.5 times greater than the control copy number. In certain embodiments, the candidate subject is known to have an amplified copy number for HER-2/neu (e.g., previously determined by immunohistochemistry, FISH, chromogenic in situ hybridization, CISH, ELISA, etc.).

The present invention further provides methods comprising; a) providing a subject with cancer, wherein the subject comprises cancer cells with an amplified copy number of HER-2/neu and topoIIα, and b) treating the subject with a topoisomerase II inhibitor. In other embodiments, the candidate subject has been previously diagnosed as having cancer cells from diseases including, but not limited to, leukemia, brain cancer, kidney cancer, lymphoma, eye cancer, connective tissue cancer, Hodgkin's disease, bone cancer, testicular cancer, cervical cancer, thyroid cancer, melanoma, skin cancer, uterine cancer, lung cancer, colon cancer, rectal cancer, ovarian cancer, bladder cancer, larynx cancer, prostate cancer, stomach cancer, breast cancer, and pancreatic cancer. In preferred embodiments, the candidate subject has breast cancer cells. In particularly preferred embodiments, the candidate subject has metastatic breast cancer cells.

The present invention also provides methods comprising: a) providing a subject with breast cancer, wherein the subject comprises breast cancer cells with an amplified copy number of HER-2/neu and topoIIα, and b) treating the subject with a topoisomerase II inhibitor. In some embodiments, the topoisomerase II inhibitor is an anthracycline. In particular embodiments, the anthracycline is selected from doxorubicin and epirubicin. In further embodiments, the breast cancer cells are metastatic. In particularly preferred embodiments, the subject is a human. In other embodiments, the subject is a non-human animal. In still further embodiments, the animal is a mammal (e.g., human, cat, dog, pig, and cow). In preferred embodiments, the animal is a female, while in other embodiments, the animal is a male.

The present invention also provides compositions comprising a probe, the probe comprising at least about 100,000 nucleotides, wherein the probe hybridizes to a target region of human chromosome seventeen under in-situ hybridization conditions, and wherein the target region contains topoIIα gene sequence, but does not contain HER-2/neu gene sequence. In preferred embodiments, the probe comprises a library of fragments ranging in size from about 0.1 kb to about 15 kb, preferably about 0.3 kb about 10 kb, and more preferably about 0.5 to about 4 kb. In certain embodiments, the probe comprises a library of fragments that hybridize to a region about 170 kb in size (e.g. 100 kb to 250 kb) containing the topoIIα gene, but does not contain the HER2 gene sequence.

In certain embodiments, the probe comprises no more than 1 million nucleotides. In other embodiments, the probe comprises no more than 500,000 nucleotides, while in other embodiments, the probe comprises no more than 250,000 nucleotides. In further embodiments, the probe comprises about 140,00 to 200,000 nucleotides (e.g. as a probe library of fragments). In preferred embodiments, the probe comprises about 170,000 nucleotides. In particular embodiments, the probe comprises at least about 125,000, 140,000, 150,000, or 160,000 nucleotides. In some embodiments, the probe contains less than ten, less than five, or less three percent repetitive nucleic acid sequences (e.g., ALU and LINE elements). In other embodiments, the probe contains less than two percent, or less than 1 percent repetitive nucleic acid sequences.

In particular embodiments, the probe further comprises a label. In certain embodiments, the label comprises digoxigenin. In other embodiments, the label is florescent. In particular embodiments, the label comprises biotin.

In certain embodiments, the target region is at least about 500,000 nucleotides from the HER-2/neu gene sequence (e.g. the site where the probe hybridizes on human chromosome 17 is at least 500,000 bases away from the HER2/neu gene). In other embodiments, the target region is at least about 400,000 or 300,000 or 200,000 nucleotides from the HER2/neu gene. In some preferred embodiments, the probe does not falsely detect HER2/neu instead of topoIIα. Also in some preferred embodiments, the target region target region comprises human chromosome locus 17q11-21.

In certain embodiments, the present invention provides kits and systems comprising the probe described above and at least one additional component. In some embodiments, the kits and systems of the present invention comprise; a) a composition comprising a probe (e.g. a library of fragments ranging in size from about 0.1 kb to about 10 kb), the probe comprising at least about 100,000 nucleotides, wherein the probe hybridizes to a target region of human chromosome seventeen under in-situ hybridization conditions, and wherein the target region contains topoIIα gene sequence, but does not contain HER-2/neu gene sequence, and b) at least one other component (e.g. insert component, primary antibody, secondary antibody, HER2 or HER2/neu probe, one or more buffers, digestion solution, cover slips, slides, graded alcohols, SSC buffer, etc). Examples 10 and 11 provide additional components for inclusion in the kits of the present invention.

In some embodiments, the insert component comprises written material. In certain embodiments, the written material comprises instructions for using the probe (e.g. in an ISH procedure such as FISH or CISH). In other embodiments, the written material comprises instructions for testing patient breast cancer tissue samples to determine if a patient should be treated with a topoisomerase II inhibitor or an anti-HER2 antibody.

In certain embodiments, the probe further comprises a label (as detailed above). In some embodiments, the kits and systems of the present invention further comprise a first antibody specific for the label (e.g., FITC-anti-digoxigenin antibody). In particular embodiments, the kits and systems of the present invention further comprise a second antibody specific for the first antibody (e.g., HRP-anti-FITC antibody).

In other embodiments, the kits and systems of the present invention further comprise a second probe, wherein the second probe specifically detects HER2 or HER2/neu. In preferred embodiments, the second probe does not falsely detect topoIIα.

In some embodiments, the present invention provides methods for performing chromogenic in-situ hybridization, comprising; a) exposing a biological sample to a enzyme digestion solution at about room temperature for about 10 minutes (e.g., 5-15 minutes, or 9-11 minutes), wherein the enzyme digestion solution comprises about 0.075% pepsin (e.g. 0.070-0.080% pepsin, with a pH of about 2.0), b) contacting the biological sample with a labeled subtracted probe library configured for detecting a target region, under conditions such that the labeled subtracted probe library hybridizes to the target region in the biological sample, c) adding a detection molecule linked to an enzyme to the biological sample under conditions such that the detection molecule binds; i) to the labeled subtracted probe library, or ii) an intermediate molecule linked to the subtracted probe library, and d) adding a calorimetric substrate to the biological sample.

In certain embodiments, the methods further comprise a step prior to step a) of preheating the biological sample in a pretreatment buffer at a temperature of at least 98 degrees Celsius. In particular embodiments, the sample is a formalin-fixed paraffin-embedded sample. In other embodiments, the methods further comprise step f) detecting the target region. In additional embodiments, the contacting comprises a stringency wash step, where the stringency wash step is performed at a temperature of at least 75° C., but not exceeding 80° C. (e.g. 75° C., 76° C., 77° C., 78° C., 79° C., and 80° C.).

In other embodiments, the present invention provides methods for performing chromogenic in-situ hybridization, comprising; a) preheating a biological sample in a pretreatment buffer at a temperature of at least 98 degrees Celsius (e.g. a FFPE biological sample), b) exposing the biological sample to a enzyme digestion solution (e.g. comprising 0.06%-0.08% pepsin), c) contacting the biological sample with a labeled subtracted probe library configured for detecting a target region selected from the group consisting of: chromosome 19q (e.g. chromosome 19q13.3-13.4), chromosome 18q (e.g. 18q21), and chromosome 1p (e.g. 1p36), under conditions such that the labeled subtracted probe library hybridizes to the target region in the biological sample, d) adding a detection molecule linked to an enzyme to the biological sample under conditions such that the detection molecule binds; i) to the labeled subtracted probe library, or ii) an intermediate molecule linked to the subtracted probe library, and e) adding a colorimetric substrate to the biological sample.

In certain embodiments, the methods further comprise step f) detecting the target region. In other embodiments, the methods further comprise step f) detecting the presence of a chromosome 19q deletion, a chromosome 18q deletion, a chromosome 22q, or a chromosome 1p deletion in the biological sample. In some embodiments, the methods further comprise step f) detecting the presence of normal diploid expression of chromosome 19q, chromosome 18q, or chromosome 1p in the biological sample.

In additional embodiments, the present invention provide methods for performing chromogenic in-situ hybridization, comprising; a) preheating a biological sample in a pretreatment buffer at a temperature of at least 98 degrees Celsius, b) exposing the biological sample to a enzyme digestion solution (e.g. comprising 0.06%-0.08% pepsin), c) contacting the biological sample with a labeled subtracted probe pair library comprising: i) a first subtracted probe library configured to hybridize to a first region of chromosome 18 that is centromeric with respect to the MALT1 gene (e.g. human MALT1 gene); and ii) a second subtracted probe library configured to hybridize to a second region of chromosome 18 that is teleomeric with respect to the MALT1 gene (e.g. human MALT1 gene), under conditions such that the labeled subtracted probe pair library hybridizes to the first and second target regions in the biological sample, d) adding a detection molecule linked to an enzyme to the biological sample under conditions such that the detection molecule binds; i) to the labeled subtracted pair probe library, or ii) an intermediate molecule linked to the subtracted probe pair library, and e) adding a calorimetric substrate to the biological sample.

In particular embodiments, the method further comprise step f) detecting the first and second target regions. In other embodiments, the methods further comprise step f) detecting the presence of the first and second subtracted probe libraries in close proximity within the cells in the biological sample, thereby indicating no translocation. In some embodiments, the methods further comprise step f) detecting the presence of the first and second subtracted probe libraries spaced apart within the cells in the biological sample, thereby indicating translocation.

In certain embodiments, the detecting comprising visualizing the calorimetric substrate with a microscope. In preferred embodiments, the microscope is a bright-field microscope. In some embodiments, the visualizing is performed with the microscope at a magnification of 35× to 45× (e.g. a dry objective with about 40× magnification is employed). In certain embodiments, the pretreatment temperature is from 98 degrees Celsius to 100 degrees Celsius.

In some embodiments, the present invention provides compositions comprising a subtracted probe library configured for detecting a target region selected from the group consisting of: chromosome 19q33, chromosome 18q21, and chromosome 1p36, wherein the subtracted probe library comprises a plurality of labeled nucleic fragments ranging in size from about 0.2 kb to about 0.5 kb, and wherein the subtracted probe library is at least 100 kb in length when hybridized to the target region. In certain embodiments the subtracted probe library, when hybridized is about 150-200 kb in length or about 50-500 kb in length.

In other embodiments, the present invention provides compositions comprising a labeled subtracted probe pair library comprising: i) a first subtracted probe library configured to hybridize to a first region of chromosome 18 that is centromeric with respect to the MALT1 gene; and ii) a second subtracted probe library configured to hybridize to a second region of chromosome 18 that is teleomeric with respect to the MALT1 gene, wherein the first and second subtracted probe libraries both comprise a plurality of labeled nucleic fragments ranging in size from about 0.2 kb to about 0.5 kb, and wherein the first and second subtracted probe libraries are each at least 100 kb in length when hybridized to the target regions. In certain embodiments, the first subtracted probe library is about 500-550 kb in length when hybridized to the first region and the second subtracted probe library is about 550-600 kb in length when hybridized to the second region.

In some embodiments, the present invention provides methods for performing in situ hybridization comprising generating a collection of probes from the chromosomal region analagous to: 1) BAC clones CTD-2307E2, CTD-2504F18, and CTD-2270N9; 2) BAC clone CTD-2571L23; 3) bk256d12, CTD-3170A23, and CTD-2021M22; 4) RP11-375I18, CTD-3051O18, and RP11-718I15; or 5) CTD-2556F3, RP11-715E11, and CTD-2559C16; and performing in situ hybridization using the collection of probes, wherein the nucleic acid molecules of the collection of probes are between 0.2 and 0.5 kb in length or between 0.1 and 50 kb in length, and wherein the collection of probes comprises less than 50%, 40%, 30%, 25%, 20%, 10%, 5%, 4%, 3%, 2%, or 1% of the highly repetitive elements in this chromosomal region. In certain embodiments, the collection of probes comprises a subtracted probe library. In particular embodiments, the collection of probes comprises at least 10, 20, 25, 50, 100, 150, 200, 250, 500, 1000, etc. nucleic acid probes. In particular embodiments, the analagous chromosomal region includes at least 3 MB (e.g. 1-3 MB) more or less of the chromosoml region that corresponds to the referenced BAC clone(s).

In certain embodiments, the present invention provides a subtracted probe library generated using subtractive hybridization (e.g. as described in the Examples below and as described in WO0026415 to Fletcher et al. or U.S. Pat. No. 6,569,621 to Craig and Cremer) on a collection of nucleic acid fragments generated using a BAC clone selected from CTD-2307E2, CTD-2504F18, CTD-2270N9, CTD-2571L23, bk256d12, CTD-3170A23, CTD-2021M22, RP11-375I18, CTD-3051O18, RP11-718I15, CTD-2556F3, RP11-715E11, and CTD-2559C16, wherein the nucleic acid molecules of the subtracted probe library are between 0.2 and 0.5 kb in length (or between 0.1 and 50 kb), and wherein the subtracted probe library comprises less than 50%, 40%, 30%, 25%, 20%, 10%, 5%, 4%, 3%, 2%, or 1% of the highly repetitive elements in this chromosomal region associated with the BAC clone(s).

In particular embodiments, the present invention provides a collection of nucleic acid probes comprising at least 10, 20, 25, 50, 100, 150, 200, 250, 500, 1000 etc. nucleic acid molecules of between 0.2 and 0.5 kb in length (or between 0.1 and 50 kb), that are complementary along at least 90% of their length to a chromosomal region analagous to a BAC clone selected from CTD-2307E2, CTD-2504F18, CTD-2270N9, CTD-2571L23, bk256d12, CTD-3170A23, CTD-2021M22, RP11-375I18, CTD-3051O18, RP11-718I15, CTD-2556F3, RP11-715E11, and CTD-2559C16, wherein the collection of probes comprises less than 50%, 40%, 30%, 25%, 20%, 10%, 5%, 4%, 3%, 2%, or 1% of the highly repetitive elements in this chromosomal region.

### DESCRIPTION OF THE FIGURES

FIG. 1 shows the results of immunohistochemical and fluorescent in situ hybridization detection in 34 primary breast cancer samples.

FIG. 2 shows the 3′ end of the Exemplary topoIIα probe (SEQ ID NO:9), and the 5′ end of the Exemplary topoIIα probe (SEQ ID NO:10).

FIG. 3 shows chart useful for interpreting ISH results using topoIIα and chromosome 17 probes.

FIG. 4 shows the BAC clones used in Example 14 that flank the ABL gene.

FIG. 5 shows ABL translocations, partner genes involved and Leukemias with ABL translocations.

FIG. 6A shows a schematic diagram of ABL DNA, and FIG. 6B shows various breakpoints in the ABL gene.

FIG. 7 shows BCR-ABL translocations.

FIG. 8 shows simplified scheme of the BCR and ABL genes with indicated breakpoints, along with exemplary BCR/ABL transcripts and proteins originating from individual breaks on the BCR and ABL genes.

FIG. 9 shows clinicopathogic correlates of the most common BCR-ABL fusions.

FIG. 10 shows UCSC genome browser for ABL gene.

FIG. 11 shows a schematic illustration of ABL translocation detection by dual-color in situ hybridization (e.g. CISH or FISH). Black dots represent ABL.c and white dots represent ABL.t. Partial karyotyptes and the corresponding interphase nuclei are shown in the figure. Normal cells without ABL translocations show black and white dots in juxtaposition, while cells with ABL translocation show one pair of black and white dots separated. Cells with ABL translocation and deletion of chromosomal material centromeric to the ABL gene breakpoint show one pair of black and white dots in juxtaposition and the black dot in another pair is disappeared (deleted).

FIG. 12 shows a clone contig map for Malt.c and Malt.t.

### Definitions

To facilitate an understanding of the present invention, a number of terms and phrases are defined below:

As used herein, the term “candidate subject”, “subject” or “patient” refers to an animal like a dog, cat, bird, livestock, and preferably a human. In some embodiments, the subject is suspected of having cancer that may be evaluated for suitability for topoisomerase II inhibitor treatment or anti-HER2 immunotherapy. Examples of subject and candidate subjects include, but are not limited to, human women suspected of having breast cancer and human men suspected of having breast cancer.

As used herein, the term “copy number” as used in reference to specific nucleic acid sequences (e.g., HER-2/neu, topoIIα and control) refers to the actual number of these sequences per single cell. Copy number may be reported for one single cell, or reported as the average number in a group of cells (e.g., tissue sample). When comparing the “copy number” of cells (e.g., experimental and control cells) one need not determine the exact copy number of the cell, but instead need only obtain an approximation that allows one to determine whether a given cell contains more or less of the nucleic acid sequence as compared to another cell. Thus, any method capable of reliably directly or indirectly determining amounts of nucleic acid may be used as a measure of copy number even if the actual copy number is not determined.

As used herein, the term “HER-2/neu” refers to a nucleic acid sequence encoding the HER2 protein, and includes both the wild-type sequence and naturally occurring variations, truncations, and mutations.

As used herein, the term “topoIIα” refers to a nucleic acid sequence encoding TopoIIα protein, or portions thereof, and includes both the wild-type sequence and naturally occurring variations, truncations, and mutations.

As used herein, the term “suitable for treatment with topoisomerase II inhibitors” when used in reference to a candidate subject refers to subjects who are more likely to benefit from treatment with topoisomersase II inhibitors than a subject selected randomly from the population. For example, using the screening methods of the present invention as described in Example 6, 79% of the subjects selected responded to topoisomerase II inhibitor treatment (as compared to 10% or less if subjects were randomly selected from the population, or as compared to approximately 30-40% of metastatic breast cancer patients).

As used herein, the term “amplification” when used in reference to copy number refers to the condition in which the copy number of a nucleic acid sequence (e.g., HER-2/neu) is greater than the copy number of a control sequence (e.g., chromosome 17). In other words, amplification indicates that the ratio of a particular nucleic acid sequence (e.g., HER-2/neu) is greater than 1:1 when compared to a control sequence (e.g., 1.1:1, 1.2:1, or 1.3:1). In preferred embodiments, the ratio of a particular nucleic acid sequence is at least 1.5 times greater than the control sequence copy number (i.e., 1.5:1).

As used herein, the term “nucleic acid molecule” and “nucleic acid sequence” refer to any nucleic acid containing molecule including, but not limited to DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “probe” refers to an oligonucleotide (i.e., a sequence of nucleotides), or a library of nucleotide fragments, whether occurring naturally as in a purified restriction digest or produced synthetically, recombinantly or by amplification (e.g. PCR), which is capable of hybridizing to an oligonucleotide of interest. Probes useful in the present invention may be single-stranded or double-stranded. Probes are useful in the detection, identification and isolation of particular gene sequences (e.g., HER-2/neu, topoIIα, and chromosome 17). It is contemplated that any probe used in the present invention may be labeled with any “reporter molecule,” so that is detectable in any detection system, including, but not limited to enzyme (e.g., ELISA, as well as enzyme-based immuno-histochemical assays), fluorescent (e.g., FISH), radioactive, mass spectroscopy, and luminescent systems. It is not intended that the present invention be limited to any particular detection system or label.

As used herein, the term “label” refers to any molecule which may be detected. For example, labels include, but are not limited to, ³²P, ¹⁴C, ¹²⁵I, ³H, ³⁵S, biotin, digoxigenin, avidin, fluorescent or enzymatic molecules.

As used herein, the phrase “repetitive nucleic acid sequences” refers to nucleic acid sequence within a genome which encompass a series of nucleotides which are repeated many times, often in tandem arrays. The repetitive sequences can occur in the genome in multiple copies ranging from two to hundreds of thousands of copies and may be clustered or interspersed on one or more chromosomes throughout a genome. Although repetitive nucleic acid sequences may be present throughout the genome, a large number of the repetitive nucleic acid sequences are typically located at the centromere of each chromosome. Examples of repetitive nucleic acid sequences include, but are not limited to, ALU and LINE elements.

As used herein, the terms “in situ hybridization” and “ISH” refer to methods for detecting and localizing nucleic acids within a cell or tissue preparation. These methods provide both quantitative and spatial information concerning the nucleic acid sequences within an individual cell or chromosome. ISH has been commonly used in many areas, including prenatal genetic disorder diagnosis, molecular cytogenetics, to detect gene expression and overexpression, to identify sites of gene expression, to map genes, to localize target genes and to identify various viral and microbial infections, tumor diagnosis, in vitro fertilization analysis, analysis of bone marrow transplantation and chromosome analysis. The technique generally involves the use of labeled nucleic acid probes which are hybridized to a chromosome or mRNA in cells that are mounted on a surface (e.g slides or other material). The probes can be labeled with fluorescent molecules or other labels. One example of fluorescent in situ hybridization (FISH) is provided in Kuo et al., Am. J. Hum. Genet., 49:112-119, 1991 (hereby incorporated by reference). Other ISH and FISH detection methods are provided in U.S. Pat. 5,750,340 to Kim et al., hereby incorporated by reference. Further examples of fluorescent in situ hybridization, as well as chromogenic in situ hybridization are provided in Examples 1-10 below. Additional protocols are known to those of skill in the art.

As used herein, the phrase “under in situ hybridization conditions” refers to any set of conditions used for performing in situ hybridization (ISH) that allows the successful detection of labeled oligonucleotide probes. Generally, the conditions used for in situ hybridization involve the fixation of tissue or other biological sample onto a surface, prehybridization treatment to increase the accessibility of target nucleic acid sequences in the sample (and to reduce non-specific binding), hybridization of the labeled nucleic acid probes to the target nucleic acid, post-hybridization washes to remove unbound probe, and detection of the hybridized probes. Each of these steps is well known in the art and has been performed under many different experimental conditions. Again, examples of such in situ hybridization conditions are provided in Kuo et al., U.S. Pat. No. 5,750,340, and Examples 1-10 (below). Further examples of conditions and reagents useful for performing in situ hybridization are provided below.

The tissue or biological sample can be fixed to a surface using fixatives. Preferred fixatives cause fixation of the cellular constituents through a precipitating action which is reversible, maintains a cellular morphology with the nucleic acid in the appropriate cellular location, and does not interfere with nucleic acid hybridization. Examples of fixatives include, but are not limited to, formaldehyde, alcohols, salt solutions, mercuric chloride, sodium chloride, sodium sulfate, potassium dichromate, potassium phosphate, ammonium bromide, calcium chloride, sodium acetate, lithium chloride, cesium acetate, calcium or magnesium acetate, potassium nitrate, potassium dichromate, sodium chromate, potassium iodide, sodium iodate, sodium thiosulfate, picric acid, acetic acid, sodium hydroxide, acetones, chloroform glycerin, and thymol.

After being fixed on a surface, the samples are treated to remove proteins and other cellular material which may cause nonspecific background binding. Agents which remove protein include, but are not limited to, enzymes such as pronase and proteinase K, or mild acids, such as 0.02.-0.2 HCl, as well as RNase (to remove RNA).

DNA on the surface may then denatured so that the oligonucleotide probes can bind to give a signal. Denaturation can be accomplished, for example, by varying the pH, increasing temperature, or with organic solvents such as formamide. The labeled probe may then hybridize with the denatured DNA under standard hybridization conditions. The tissue or biological sample may be deposited on a solid surface using standard techniques such as sectioning of tissues or smearing or cytocentrifugation of single cell suspensions. Examples of solid surfaces include, but are not limited to, glass, nitrocellulose, adhesive tape, nylon, or GENE SCREEN PLUS.

As used herein, the term “polymerase chain reaction” (“PCR”) refers to the method described in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the phrase “anti-HER2 antibody-free topoisomerase II inhibitor treatment” refers to a treatment regimen for a subject that includes administering topoisomerase II inhibitors (e.g. anthracyclines), but does not include anti-HER2 antibody administration at about the same time.

As used herein, the phrase “topoisomerase II inhibitor-free anti-HER2 antibody treatment” refers to a treatment regimen for a subject that includes the administration of anti-HER2 antibodies (e.g. HERCEPTIN), but does not include topoisomerase II inhibitor (e.g. anthracyclines) administration at about the same time.

As used herein, the phrase “subtracted probe library” refers to a mixture of nucleic acid fragments configured to hybridize to a target region (e.g. selected portion of a chromosome containing gene of interest) that comprises at least about 90 percent repeat free fragments.

### DESCRIPTION OF THE INVENTION

The present invention relates to chromogenic (calorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, and chromosome polysomy). In preferred embodiments, the present invention provides CISH methods, kits and compositions for detecting HER2 gene status. The description of the invention is presented below in the following sections: I. Chromogenic In-Situ Hybridization; II. CISH HER-2/neu Detection and Anti-HER2 Antibody Therapy; III. Combined HER2/HER-2/neu and topoIIα detection; IV. Combined CISH and IHC; V. Subtracted Probes; VI. ABL Probe Pairs and Detecting BCR-ABL Translocations; VII. MALT1 Probe Pair; VIII) Chromosome 19q deletion probe and Chromosome 1p deletion probe; IX) Chromosome 18q deletion probe; and X) NF2 (Chromosome 22q) deletion probe.

I. Chromogenic In Situ Hybridization

Chromogenic in situ hybridization (CISH) is a technique that allows in situ hybridization methods to be performed and detected with a bright-field microscope, instead of a fluorescence microscope as required for FISH. While FISH requires a modern and expensive fluorescence microscopes equipped with high-quality 60× or 100× oil immersion objectives and multi-band-pass fluorescence filters (not used in most routine diagnostic laboratories), CISH allows detection with standard light (bright-field) microscopes (which are generally used in diagnostic laboratories). Also, with FISH, the fluorescence signals can fade within several weeks, and the hybridization results are typically recorded with an expensive CCD camera, while the results of CISH do not generally fade allowing the tissue samples to be archived and reviewed later. Therefore, analysis and recording of FISH data is expensive and time consuming. Most importantly, tissue section morphology is not optimal in FISH on FFPE. Generally, histological detail is better appreciated with bright-field detection, which is possible with CISH detection. A further advantage of CISH is that large regions of tissue section can be scanned rapidly after CISH counterstaining since morphological detail is readily apparent using low power objectives (e.g. 10× and 20×), while FISH detection generally requires substantially higher magnification (thus reducing the field of view). These advantages generally make CISH a superior in situ hybridization technique compared to FISH.

General chromogenic/colorimetric in situ hybridization methods are described in WO0026415 to Fletcher et al. (herein incorporated by reference for all purposes). Particular reagents and steps for performing CISH on formalin-fixed, paraffin-embedded (FFPE) tissue samples, as well as cell sample/metaphase chromosome samples are described in WO0026415 and the section presented below. Importantly the description detailed below provides exemplary CISH methods, procedures, and reagents, and is not to be construed as limiting the present invention.

A. Formalin-Fixed, Paraffin-Embedded (FFPE) Tissue Samples

Generally, FFPE tissue samples (e.g. cancer biopsy tissue samples) will measure about 1-2 cm in diameter, but any type of diameter may be employed. This tissue sections (e.g. 4-5 um) may be mounted on treated (e.g. HISTOGRIP treated) microscope slides or other solid support surface (e.g. Superfrost/Plus microscope slides).

i. Pretreatment

In preferred embodiments, the FFPE tissue samples are first subjected to a deparaffinization step. This may be accomplished, for example, by exposing the sample to Xylene for about 10 minutes at room temperature. This may be repeated if necessary. The sample may then be exposed to EtOH (e.g. 100% EtOH) for about 5 minutes at room temperature. In preferred embodiments, this is performed three times. The tissue samples are then allowed to dry (e.g. air dry).

Next, tissue samples are subjected to a heat pretreament step. Specifically, a pretreatment buffer is added to the tissue samples, and the samples are heated to approximately 92-100 degrees Celsius for approximately 15 minutes (although varying incubation times may be used depending on the tissue fixation). Examples of pretreatment buffers included, but are not limited to, Citrate buffer, EDTA-TRIS buffer (e.g. 0.1M Tris/0.05 M EDTA, pH 7.0), and TRIS buffer. In certain embodiments, the preheat temperature is achieved with a microwave, a pressure cooker, a hot plate, or other type of heating device. Also, in preferred embodiments, the preheat temperature is such that the pretreatment buffer boils. For example, a preferred temperature range is 96-100 degrees Celsius. A particularly preferred temperature range is 98-100 degrees Celsius. It was determined that the temperature range of 98-100 gives enhanced CISH detection results (e.g. as compared to 92 degrees Celsius). The tissue samples are then generally washed (e.g. with water or PBS) two or three times (e.g. for 2-4 minutes per wash).

Generally, the next step is an enzyme digestion step. In preferred embodiments, the tissue samples are exposed to pepsin digestion (e.g. at room temperature or at about 37° C.) for about a several minutes (e.g. 1-20 minutes may be required depending on tissue fixation). In preferred embodiments, the enzyme digestion solution comprises about 0.075% pepsin (e.g. 0.070-0.080% pepsin) at pH 2.0 and the enzyme digestion occurs for about 10 minutes at about room temperature. Importantly, excessive digestion may cause loss of nuclei and chromosome structure, while inadequate digestion may result in loss of signal. The tissue samples are then washed again (e.g. with water or PBS) two or three times (e.g. for 2-4 minutes per wash).

After washing, the tissue samples are then dehydrated with graded alcohols. For example, the tissue samples may be exposed to 70%, 85%, 95%, and 100% ethanol for about 2 minutes each time, and then air dried.

ii. Denaturation and Hybridization

Denaturation and hybridization may accomplished as one step (co-denaturing and hybridization, described in this paragraph), or as two steps (separate denaturation and hybridization, described below). One general procedure for co-denaturation and hybridization is as follows. First, add the probe (e.g. 12-20 ul of a subtracted probe library) to the center of a cover slip (e.g. 22×22 mm coverslip, or 24×32 mm coverslip, or coverslips described in WO0138848 to Ventana Medical Systems Inc., herein incorporated by reference). In other embodiments, the probe is added directly to the tissue sample. In other embodiments, the liquid COVERSLIP from Ventana Medical Systems, Inc. is applied over the tissue sample (e.g. to create a humid reaction chamber on the slide). In other embodiments, the Zymed CISH UNDERCOVER slips are employed (available from Zymed Labs.). In some embodiments, the coverslip is then placed probe side down on the tissue sample. The edges of the coverslip may then be sealed, for example, with a thin layer of rubber cement to prevent evaporation during incubation. The slide with the tissue sample is then placed on a slide block of a PCR machine or on a heating block with temperature display (or other heating device, such as a ThermoBrite device from StatSpin). Denaturation may be conducted at approximately 94-95 degrees Celsius for about 5-10 minutes, at about 85-90 degrees Celsius (e.g. on a ThermoBrite device), or anywhere between about 75 and 100 degrees Celsius. The tissue sample (e.g. on the slide) is then incubated at approximately 37 degrees Celsius for about 16-24 hours. Incubation may be conducted, for example, in a dark humidity box (or other humidified chamber) or in the slide block of a PCR thermal cycler.

One general procedure for separate denaturation and hybridization is as follows. This procedures is useful, for example, when a PCR machine or heating block are not readily available. First, the tissue sample is denatured in denaturing buffer (e.g. 4 ml 20×SSC [20×SSC buffer=0.3M Sodium Citrate, with 3M NaCl, ph 7.0], 8 ml ddH₂O, 28 ml formamide) at about 75 degrees Celsius for about 5 minutes. Increases in temperature may be used for additional samples being denatured at the same time (e.g. add about 1 degree Celsius for each additional sample being denatured). Next, the slides are denatured with graded alcohols (e.g. 70% EtOH, 85% EtOH and 95% all for about 2 minutes at negative 20 degrees Celsius, and then 100% EtOH for about 2 minutes twice).

Then the tissue samples are air dried, while the labeled probe (e.g. subtracted probe) is denatured at about 75 degrees Celsius for about 5 minutes. The denatured probe is then placed on ice. About 12-15 ul of the denatured probe is added to the center of a coverslip (e.g. a 22×22 mm coverslip, or other cover). The coverslip is then added to the appropriate tissue sample area, and the tissue sample is placed in a dark humid box (or other humidified chamber) at about 37° C. for at least about 14 hours. Next step, for example, would be the stringency wash below.

B. Cell Sample or Metaphase Chromosome Sample

i. Pretreatment

Initially, slides may be immersed in a pretreament buffer such as 2×SSC buffer (20×SSC buffer=0.3M Sodium Citrate, with 3M NaCl, ph 7.0), or Tris-EDTA, or Tris, at about 37 degrees Celsius for about 60 minutes. In some embodiments, the cell samples are treated with pepsin compositions (e.g. Zymed's SPOT LIGHT Cell Pretreatment Reagent) for about 5 minutes at about 37 degrees Celsius. Incubation time may be, for example, from about 1-10 minutes depending on cell type and slide-making conditions. Excessive pepsin digestion may cause loss of nuclei and chromosome structure. Inadequate digestion may result in loss of signal. Slides may then be washed (e.g. in dH₂O or PBS) for two or three time, for two or three minutes each time at room temperature. In some embodiments, the slides may be immersed in buffered formalin (e.g. 10%) for about a minute at room temperature. The slides may then be washed (e.g. in dH₂O or PBS) two or three times for about 1-3 minutes each time, at room temperature. The slides may then be dehydrated. For example, the slides may be dehydrated in 70%, 85%, 95%, and 100% ethanol for 2 minutes each, and then air dried. Slides may proceed to ISH procedures described below or stored (e.g. in 70% ethanol at −20 degrees Celsius).

ii. Denaturation and Hybridization

First, add the probe (e.g. 12-20 ul of a subtracted probe library, See Subtracted Probe section below) to the center of a cover slip (e.g. 22×22 mm coverslip, or 24×32 mm coverslip, or coverslips described in WO0138848 to Ventana Medical Systems Inc., herein incorporated by reference). In other embodiments, the probe is added directly to the tissue sample. In some embodiments, the liquid COVERSLIP from Ventana Medical Systems, Inc. is applied over the tissue sample (e.g. to create a humid reaction chamber on the slide). In other embodiments, the Zymed CISH UNDERCOVER slips are employed (available from Zymed Labs.). In some embodiments, the coverslip is then placed probe side down on the tissue sample. The edges of the coverslip may then be sealed, for example, with a thin layer of rubber cement to prevent evaporation during incubation. For denaturation, the slide with the tissue sample is then placed on a slide block of PCR machine or on a heating block with temperature display (or other heating device). Denaturation is conducted at approximately 80 degrees Celsius for about 2-5 minutes. The slides may then be placed in a dark humidity box (or other humidity chamber) or in the slide block of a PCR thermal cycler for about 16-24 hours at about 37 degrees Celsius.

iii. Stringency Wash

The remaining steps (e.g. stringency wash, immunodetection, counterstaining/coverslipping) are generally the same for both cell sample and FFPE. After hybridization, the rubber cement (or other sealant used, if a sealant is used) and cover slip (or other cover) is carefully removed. The tissue sample slides are then washed (e.g. in Coplin jar) in order to remove unhybridized probes. For example, the tissue sample slides may be washed in 0.5×SSC at 72° C. for about 5 minutes. The temperature may be adjusted up if more than one slide is being washed (e.g. add 1° C. per slide for more than 2 slides, but preferable no higher than 80° C. The slides are then washed again in, for example, dH₂O or PBS/Tween 20 buffer for about 2-3 minutes. This may be repeated two or three times. In certain preferred embodiments, the stringency wash is conducted at a temperature of at least 75° C., but not more than 80° C.

iv. Immunodetection

Generally, depending on the detection reagents used, the first step in preparation for immunodetection is peroxidase quenching and endogenous biotin blocking. For peroxidase quenching, slides may be submerged in 3% H₂O₂ in absolute methanol (e.g. add part 30% hydrogen peroxide to 9 parts absolute methanol) for about 10 minutes. The slide is then washed with PBS (e.g. 1×PBS (10 mM)/Tween 20 (0.025%)) for 2-3 minutes. This may be repeated two or three times. The tissue samples are then blocked. Blocking can be performed by adding 2 drops per slide (at room temperature) of CAS-BLOCK (which is 0.25% casein, 0.2% gelatin, and 10 mM PBS, pH 7.4). After about 10 minutes, the blocking reagent is blotted off.

Next, the labeled probe library is detected. The probe may be detected by first adding an anti-label primary antibody (e.g. a mouse antibody or antibody with a label such as FITC). In certain preferred embodiments, the probe is labeled with digoxigenin, and the primary antibody is an FITC-anti-dig antibody. In other preferred embodiments, the primary antibody is unlabelled, but is from a particular species such as rat, mouse or goat. In other embodiments, the primary antibody is linked (e.g. conjugated) to an enzyme (e.g. horseradish peroxidase (HRP) or alkaline phosphatase (AP)) able to act on a chromogenic substrate, and does not require the secondary antibody described below. Generally, about two drops of the primary antibody solution is added to the tissue at room temperature for about 30-60 minutes. The tissue sample is then rinsed, for example, with PBS (e.g., 1×PBS/Tween 20 (0.025%) for about 2-3 minutes. This may be repeated two to three times.

In preferred embodiments, a secondary antibody is added to the tissue sample that is able to bind to the primary antibody. For example, if the primary antibody is labeled with FITC, the secondary antibody may be an anti-FITC antibody. Also for example, if the primary antibody is an unlabeled mouse antibody, the secondary antibody may be an anti-mouse antibody (e.g. goat anti-mouse antibody). Generally, the secondary antibody is linked (e.g. conjugated) to an enzyme (e.g. HRP or AP) able to act upon a chromogenic substrate (or chemiluminescent substrate). Generally, about 2 drops of the secondary antibody is added to the tissue sample at room temperature for about 30-60 minutes. The tissue sample is then rinsed, for example, with PBS (e.g., 1×PBS/Tween 20 (0.025%) for about 2-3 minutes. This may be repeated two to three times. Additional antibodies (e.g. tertiary, quaternary antibodies) may be used if desired.

In certain preferred embodiments, the secondary antibody is linked to a polymer that is itself linked to many enzyme molecules (e.g. polymerized HRP or polymerized AP). This allows each individual antibody to connect (via the polymer) to many enzyme molecules in order to increase signal intensity. Such polymerized enzymes are known in the art, and are commercially available from, for example, Nichirei Inc. (Tokyo, Japan) and ImmunoVision.

Once the antibody (or other detection molecule) which is linked to an enzyme (e.g. a secondary or tertiary antibody conjugated to AP or HRP), is added to the biological sample, a substrate for the enzyme is then added. In preferred embodiments, the substrate is a chromogen. Examples of suitable chromogens include, but are not limited to, DAB, FAST RED, AEC, BCIP/NBT, BCIP/INT, TMB, APPurple, ULTRABLUE, TMBlue, and VEGA RED. In other embodiments, the substrate is a chemiluminescent molecule (e.g. BOLD APS 540 chemiluminescent substrate, BOLD APS 450 chemiluminescent substrate, or BOLD APB chemiluminescent substrate, all commercially available from INTERGEN Co.). Therefore, the next step, for example in developing the slide, is to mix DAB (or other substrate), buffer, and hydrogen peroxide (e.g. 0.6%) in a tube, then to add 3 drops per slide to the tissue sample for about 30 minutes. In certain embodiments, chromogen enhancers are added to increase signal intensity (e.g. AEC enhancer, FAST RED enhancer, and DAB enhancer available from INNOVEX Biosciences, ZYMED Labs, etc.). The tissue sample may then be washed (e.g. with running tap water) for about two minutes. In certain embodiments, the immunohistochemistry steps are automated or partially automated. For example, the ZYMED ST 5050 Automated Immunostainer or the ZYMED MOZAIC instrument may be employed to automate this process.

v. Counterstaining and Coverslipping

In some embodiments, the next step is a counterstaining and coverslipping step. This step may be performed by counterstaining the tissue sample. For example, the tissue sample may be counterstained with hematoxylin or other counterstain. This procedure may be performed for about 6 seconds to about 1 minutes, depending on the type of tissue being stained. Preferably, overly dark counterstaining is avoided so as not to obscure the positive signal. The slides may then be washed (e.g. with running tap water) for a couple of minutes, and then, in some embodiments, dehydrated with graded EtOH (e.g. 70%, 85%, 95%, 100%, 100% for about 2 minutes each, repeated two times). In some embodiments, the dehydration is not performed with EtOH, when, for example, FAST RED is the substrate (e.g. a water soluble substrate). The slides may then be exposed to Xylene for about two minutes (this may be repeated at least once). The tissue sample may then be coverslippped (e.g. with HISTOMOUNT, Cytoseal 6.0, cat. # 8310-16, Stephen Scientific). In some embodiments, CLEARMOUNT is employed instead (e.g. when FAST RED is one of the substrates).

vi. Microscopy and Interpretation of Results

Importantly, the slides may be visualized using standard bright-field microscopy using a bright-field microscope (e.g. OLYMPUS, NIKON, LEITZ, etc.). Generally, probes are visible with about 20× magnification (e.g. 15×-25×). In preferred embodiments, probes are visualized with about 30×, or 40× (e.g. 28×-43×) magnification. Higher powers (e.g. 60×, 80×, and 100×) may be employed, but are generally not necessary (and may reduce the field of view). In some embodiments, for evaluating translocation results, a 100× oil lens is employed. In other embodiments, for evaluating amplification and centromere probes, 40× lens is employed. Below are examples of how CISH results may be interpreted for gene amplification/centromere detection, as well as for gene translocation.

As mentioned above CISH detection of gene amplification, translocation, and cetromere detection may be performed with a bright-field microscope, or other type of microscope. For example, in general, CISH staining results are clearly seen using a 40× objective in tissue sections which are counterstained (e.g., hematoxylin). An individual gene or chromosome centromere signal normally appears as a small, single dot. Targeted gene amplification is typically seen as large chromogen-stained (e.g. DAB-stained) clusters or many dots in the nucleus or mixed clusters and multiple dots (e.g., ≧6 dots per nucleus). Tumors with no targeted gene amplification typically show 1 to 5 dots per nucleus. Normally, 3-5 dots per nucleus in more than 50% of tumor cells are due to chromosome polysomy. Table 1 shows an exemplary chart useful for CISH visualization of individual genes for chromosome polysomy. Example 22 also contain a useful table for CISH visualization.

TABLE 1
Exemplary CISH Signal Visualization for an individual gene or
chromosome centromere

| Magnification | CISH Signal |
|---|---|
| 10× | Individual signals are barely visible and may be |
| | missed. |
| 20× | Individual signals are small but clearly discernible. |
| 40× | Individual signals are easily identified. |
| 60× or 100× | Not Necessary |

Examples of CISH detection and interpretation of gene amplification in HER2 and TopoIIα CISH, are presented in Tables 2 and 3 below.

TABLE 2
Exemplary Assessment of HER2 gene status by CISH

| | |
|---|---|
| Amplification | >10 copies or large clusters of HER2 gene (amplicon) per |
| | nucleus in >50% of cancer cells. |
| Low | 6-10 copies of HER2 gene or small cluster of HER2 |
| Amplification | gene (amplicon) per nucleus in >50% of |
| | cancer cells. |
| | Labeled chromosome 17 centromere probe may be |
| | applied for CISH to confirm that 6-10 copies of |
| | HER2 gene (<5% cases) were due to HER2 gene |
| | amplification but not chromosome 17 polysomy. |
| No | 1-5 copies of HER2 gene per nucleus in >50% of |
| Amplification | cancer cells. |
| | 3-5 copies of HER2 gene per nucleus is |
| | due to chromosome 17 polysomy. There is no |
| | need for chromosome 17 centromere CISH. |
| | Occasionally, it is found that HER2 has 3-5 copies and |
| | chr.17cen has 1-2 copies in >50% of cancer cells |
| | (HER2/chr.17cen ratio is ≧2), it is due to what |
| | sometimes was seen by CGH of duplication of |
| | chromosome arm 17q. |

TABLE 3
Exemplary Topo IIα Probe and Chromosome 17 Centromeric Probe Usage

| Topo IIα | | Chromosome 17 |
|---|---|---|
| Status | Topo IIα Results | Centromeric Probe |
| Deletion | When Topo IIα gene copy number is less than the | |
| | centromeric copy number. | |
| Normal diploid | 2 copies | 2 copies |
| Aneuploidy | 3-5 copies | 3-5 copies |
| Amplification | Gene cluster (amplicon) | Gene amplification is highly |
| | or ≧6 separate | likely, Chromosome 17 |
| | copies | Centromeric Probe analysis |
| | | is not necessary |

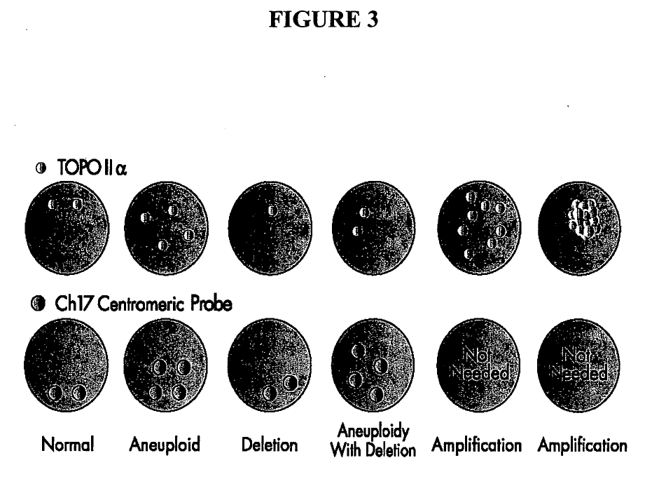

Also, in some normal cells, one gene copy may be missing due to loss of nuclear material during sectioning. Therefore, in general, analysis should be based on the results from the majority of cancer cells (>50%) observed. FIG. 3 presents one interpretation chart for interpreting topoIIα amplification using topoIIα and chromosome 17 centromere probes. It should be noted that these are representative examples only. Copy numbers from actual samples may vary for aneuploidy, deletion, and amplification.

vii. Quality Control Procedures

In some embodiments, quality control procedures are used. Quality control over the accuracy of the above procedures may, in some embodiments, be assured by using some or all of the controls described below.

Positive Tissue Control:

External positive control materials for clinical research generally should be fresh autopsy/biopsy/surgical specimens fixed, processed, and embedded as soon as possible in the same manner as the patient sample(s). Specimens processed differently from the specimen sample(s) validate reagent performance, and do not verify tissue preparation. Positive tissue controls are indicative of correctly prepared tissues and proper staining techniques. One positive tissue control for each set of test conditions may be included in each run. For example, for topoIIα gene detection, tissues used for the positive control materials should be selected from specimens with well-characterized levels of topoIIα gene. Approximately 5-10% of breast cancer tissue has topoIIα gene amplification and may be a useful source of positive control tissue.

Known positive controls may be utilized for monitoring the correct performance of processed tissues and test reagents, rather than as an aid in interpreting sample results. If the positive tissue controls fail to demonstrate positive staining, results with the specimen samples should generally be considered invalid.

Negative or Normal (Diploid) Tissue Control:

Normal tissue can be used as a negative control for gene amplification or deletion. Use a negative tissue control (known to be diploid) fixed, processed, and embedded in the same manner as the sample(s) with each staining run. This will verify the specificity of the ISH probe, and provide an indication of non-specific background staining (false positive staining).

A negative tissue control that is separate from the sample is known as an ‘external’ negative control. If an external negative tissue control is not available then a normal section of the sample can serve as an ‘internal’ negative tissue control.

In certain embodiments, the negative tissue control is examined after the positive tissue control to verify the specificity of hybridization. Generally, the presence of no more than two gene copies in most of the cells in the negative tissue control confirms that the probe and detection reagents are not cross-reacting with cellular or tissue components. Occasionally, 0, 1, 3, or 4 gene copies may be seen in the nucleus. Normal tissue counterparts in an abnormal sample may also be used as negative tissue controls. If non-specific straining occurs in the negative tissue control, results obtained for the sample specimen(s) should generally be considered invalid. Also, non-specific staining usually exhibits a diffuse staining pattern. Sporadic staining of connective tissue may also be observed in sections from excessively formalin-fixed tissues. In preferred embodiments, normal cells are used for interpretation of staining results as necrotic or degenerated cells often stain non-specifically.

Reagent (No-Probe) Control:

A reagent control may be run on a section of sample specimen without the probe. The reagent control is useful in evaluating the possibility of nonspecific staining, particularly when performing ISH in tissue sections. The reagent control may be stained in the same way as the test samples except that hybridization buffer, that does not contain the probe, should generally be used during the hybridization step. Slide pretreatment, denaturation, and immunodetection should generally be performed under the same conditions as test samples.

viii. Automation

In certain preferred embodiments, all or part of the procedures described above for performing CISH are automated. Automation is useful for high throughput processing of many samples (e.g. in a clinical lab). Examples of methods and devices useful for such automation are found in WO9943434, and WO9944030 to Ventana Medical Systems Inc., (Tucson, Ariz.) both of which are herein incorporated by reference. Additional examples are automated in situ hybridization and immunohistochemistry devices commercially available from Ventana Medical Systems Inc, such at the BENCHMARK in-situ hybridization module. In other embodiments, Cytologix Corp. (Cambridge Mass.) equipment is used (e.g. as shown in WO0063670, WO0062064, WO9944032, WO9944031, and WO9901770, all of which are herein incorporated by reference).

II. CISH HER-2/neu Detection and Anti-HER2 Antibody Therapy

The present invention also provides methods, kits, and compositions for detecting HER2 gene amplification (e.g. on a patient) sample using CISH (See, e.g, Examples 7 and 8). Once HER2 gene amplification is detected by CISH, the patient from which the sample is derived is then able to be identified as a good candidate to receive anti-HER2 antibody immunotherapy. In some embodiments, the patient is prescribed anti-HER2 antibodies. In other embodiments, the patient is administered a therapeutic dose or doses of anti-HER2 antibodies (e.g. chimeric, humanized, or fully human anti-HER2 antibodies).

Examples of antibodies which bind HER2 include, but are not limited to, MAbs 4D5 (ATCC CRL 10463), 2C4 (ATCC HB-12697), 7F3 (ATCC HB-12216), and 7C2 (ATCC HB12215) (see, U.S. Pat. No. 5,772,997; PCT Publication No. WO 98/77797; and U.S. Pat. No. 5,840,525, all of which are expressly incorporated herein by reference). Examples of humanized anti-HER2 antibodies include, but are not limited to, huMAb4D5-1, huMAb4D5-2, huMAb4D5-3, huMAb4D5-4, huMAb4D5-5, huMAb4D5-6, huMAb4D5-7, and huMAb4D5-8 (HERCEPTIN & commat;) as described in Table 3 of U.S. Pat. No. 5,821,337, which is expressly incorporated herein by reference; and humanized 520C9 (PCT Publication No. WO 93/21319, herein incorporated by reference). Examples of human anti-HER2 antibodies include, but are not limited to, those that are described in U.S. Pat. No. 5,772,997 and PCT Publication No. WO 97/00271, both of which are herein incorporated by reference.

III. Combined HER2/HER-2/neu and topoIIα Detection

The present invention provides methods for diagnosing and treating cancer, and in particular, methods for determining the susceptibility of subjects suspected of having breast cancer (or known to have breast cancer) to treatment with topoisomerase II inhibitors and treatment with anti-HER2 antibody therapy. Importantly, the present invention provides methods, compositions, and kits for detecting copy number for both topoIIα and HER-2/neu, or detecting HER2 expression (e.g. overexpression) and topoIIα copy number (e.g. amplification) which leads to improved diagnostic treatment procedures (e.g. for successfully treating breast cancer patients). For example, given the dangers associated with the co-administration of topoisomerase II inhibitors (such as anthracyclines) and anti-HER2 antibodies (e.g. HERCEPTIN), the present invention provides methods for selecting which treatment is likely to be useful for a particular patient. This is accomplished, in some embodiments, by determining a copy number for both topoIIα and HER2/neu in a tissue sample from a patient (e.g. breast cancer patient), or detecting a copy number for topoIIα and expression levels of HER2.

In some embodiments, the present invention provides methods for determining whether a subject suspected of having breast cancer would benefit from treatment with topoisomerase II inhibitors (e.g., anthracyclines). For example, the present invention provides diagnostic assays for detecting an amplified copy number of HER-2/neu and topoIIα in breast cancer cells of a candidate subject, and identifying whether the candidate subject is suitable for treatment with topoisomerase II inhibitors (e.g. without concomitant anti-HER2 antibody therapy), or treatment with anti-HER2 antibody therapy (e.g. without concomitant topoisomerase II inhibitor therapy such as anthracycline therapy). In other embodiments, the present invention provides methods for treating breast cancer by administering topoisomerase II inhibitors (e.g., anthracyclines) to subjects, with breast cancer cells with an amplified copy number of HER-2/neu and topoIIα. For ease in reading, this section is divided into the following sections: A. Breast Cancer; B. Treatment for Metastatic Breast Cancer; C. TopoIIα and TopoIIα; D. Detection of TopoIIα; E. HER-2 and HER-2/neu; F. Detection of HER2 and HER-2/neu; G.; HER-2/neu-TopoIIα Relationship; and H. HER-2/neu-TopoIIα Status as Diagnostic Marker.

A. Breast Cancer

Despite earlier diagnosis of breast cancer, about 1-5% of women with newly diagnosed breast cancer have a distant metastasis at the time of the diagnosis. In addition, approximately 50% of the patients with local disease who are primarily diagnosed eventually relapse with the metastasis. Eighty-five percent of these recurrences take place within the first five years after the primary manifestation of the disease.

On presentation, most patients with metastatic breast cancer have only one or two organ systems involved. As the disease progresses over time, multiple sites usually become involved. Indeed, metastases may be found in nearly every organ of the body at autopsy. The most common sites of metastatic involvement observed are locoregional recurrences in the skin and soft tissues of the chest wall, as well as in axilla, and supraclavicular area. The most common site for distant metastasis is the bone (30-40% of distant metastasis), followed by lung and liver. Metastatic breast cancer is generally considered to be an incurable disease. However, the currently available treatment options often prolong the disease-free state and overall survival rate, as well as increase the quality of the life. The median survival from the manifestation of distant metastases is about three years.

In some patients, advanced disease can be controlled with therapy for many years allowing good quality of life. This is particularly evident for those patients with hormone receptor positive disease and nonvisceral sites of metastases. It is contemplated that with better understanding of the molecular factors involved in the response to chemotherapy and increased efficiency of chemotherapy, regimens will substantially extend the survival for these patients, and in some patients, perhaps even extend survival to their otherwise natural life-span. However, despite these promises, the current reality is that treatment provides only temporary control of cancer growth for most patients with metastatic breast cancer.

B. Treatment for Metastatic Breast Cancer

Systemic drug therapy for advanced breast cancer is usually started with hormonal therapy due to its lower toxicity than the cytotoxic chemotherapies. The best candidates for hormonal therapy, based on their clinical features, are patients with a hormone receptor positive tumor (especially when both hormone receptors are positive), long term disease free survival, previous response to hormonal therapy, and non-visceral disease. Despite short second-line and even third-line responses to alternative hormonal therapies (e.g., second anti-estrogen or aromatase inhibitor) in advanced stage of breast cancer, nearly all patients finally become refractory to hormonal therapy and their disease progresses.

Due to its higher toxicity, cytotoxic chemotherapy is given to patients with disease refractory to hormonal therapy. In addition, it is frequently used as the first-line therapy for those with extensive visceral involvement of metastatic disease (e.g., lung or liver metastasis), with hormone receptor negative primary tumor, with extensive involvement of bone marrow, or with tumor that is so rapidly growing that the response to hormonal therapy can not be monitored. Combination chemotherapy for advanced breast cancer is generally considered more efficacious than single-agent therapy. However, randomized trials have shown that similar response rates can be achieved with single-agent therapy.

Advanced breast cancer is currently considered to be incurable and nearly all available chemotherapeutic drugs have been tested for use in its treatment. Among the large number of cytotoxic drugs, anthracyclines (which are topoII-inhibitors), especially doxorubicin and its derivative epirubicin, and taxanes are considered to be the most efficacious. The optimal schedules for the newer drugs, paclitaxel and docetaxel (taxanes), are yet to be established.

In addition to anthracyclines, other topoII-inhibitors include cytotoxic agents such as etoposide, amsacrine, and mitoxantrone. All these agents target the topoisomerase IIα enzyme (topoIIα) and are now routinely employed in the systemic treatment of hematological cancers and solid tumors. Generally, the chemotherapeutic regimens for the most curable malignancies, such as lymphomas and leukemias, as well as for breast cancer are based on such agents that act on topoIIα.

In the treatment of breast cancer, these compounds are not only given for patients with metastatic disease, but are also gaining popularity as a foundation for adjuvant chemotherapy regimens. Whether given alone or combined with other cytotoxic drugs, the objective response rate to anthracyclines generally ranges from 40% to 80% in metastatic breast cancer. However, the rate of complete response is approximately 5-15% and usually lasts for one to two years in these patients. The proportion of patients who achieve complete, prolonged (i.e., several years) remissions is below 1%. More typically, the response is partial (50% reduction in tumor mass) and its duration ranges from 6 to 12 months. Thus, there is still a large number of patients who do not receive objective, clinical response to these cytotoxic drugs. In these patients the disease progression may just be halted or continue to progress despite the treatment. About 40-60% of the breast cancer patients receiving anthracyclines have either stabilized or develop progressive disease during the therapy. Therefore, there is a need for reliable selection of patients who are likely to respond to therapy from those likely to have primary resistance to anthracyclines.

As important as it is to identify the patients likely to respond to therapy, it may be even more relevant to identify patients who are not likely to achieve any objective response to anthracyclines, because the tumors resistant to anthracyclines also acquire resistance to other classes of cytotoxic drugs during anthracycline therapy (i.e., the tumors become multi-drug resistant (MDR)). The MDR phenotype turns cancer cells resistant to virtually any form of cytotoxic chemotherapy (excluding the taxanes). Indeed, MDR tumor cells are even resistant to agents with no functional or mechanistic interaction with topoII-inhibitors.

The most recent breakthrough in the treatment of human malignancies has been the introduction of monoclonal antibodies which specifically target genes that are involved in the pathogenesis of cancer. The first such antibody targeting human oncogene is called Trastuzumab (HERCEPTIN, Genentech BioOncology, Roche), and was introduced to the treatment of breast cancer patients in 1997. HERCEPTIN specifically binds the extracellular domain of the HER-2 and abolishes growth factor signaling through HER-2 and other growth factor receptors attached to HER-2.

In clinical trials, HERCEPTIN was shown to be generally well tolerated with the most common adverse effects being chills and fever in approximately 40% of patients (mainly associated with the first infusion). However, when administered in conjunction with anthracyclines, HERCEPTIN resulted in an increased risk of cardiac dysfunction in patients. In particular, it has been reported that 27% of patients receiving combined therapy with HERCEPTIN and anthracyclines experienced cardiac dysfunction, while only 6% of patients receiving anthracycline therapy alone experienced cardiac dysfunction. Thus, the present invention provides methods for identifying candidate subjects that would benefit from anthracycline therapy, even though they may initially be viewed as HERCEPTIN therapy candidates. The present invention also provided methods (e.g. dual topoIIα and HER2/neu testing) to identify patients that should receive HERCEPTIN without also receiving anthracyclines (or other topoIIα inhibitors).

C. TopoIIα and TopoIIα

Topoisomerases are enzymes involved in resolving topological problems that arise during the various processes of DNA metabolism, including transcription, recombination, replication, and chromosome segregation during cell division. As a result of performing these vital functions, topoisomerases are necessary for the viability of all living organisms.

Topoisomerases are classified into “Type I” and “Type II” based on their catalytic activity. Type I enzymes introduce transient single-stranded breaks into DNA, pass a single intact strand of DNA through the broken strand, and re-ligate the break. Type II enzymes, in contrast, make transient double-stranded breaks in one segment of replicated DNA and pass an intact duplex through the broken double-stranded DNA.

Among different topoisomerase enzymes, type II DNA topoisomerases (topoII) are essential in the segregation of newly replicated chromosome pairs, chromosome condensation, forming chromosome scaffolds, and altering DNA superhelicity. The reaction of transporting the intertwined double-stranded DNA through a double-stranded break favors a “two-gate model”. In this model, topoII forms an ATP-operated clamp through which the first segment of DNA binds and which then captures the DNA segment to be transported. Once the transported segment has passed through the break in the bound DNA, it is allowed to leave the enzyme by another gate on the other side of the molecule, while the double-stranded break in the bound DNA is simultaneously re-sealed by the enzyme. Consistent with this biochemical model of the enzyme as an ATP-modulated clamp with two sets of jaws at opposite ends, connected by multiple joint, the crystal structure of topoII reveals a heart-shaped dimeric protein with a large central hole.

The eukaryotic topoII is a homodimeric enzyme that exists in two isoforms in human cells, the major, 170-kd topoIIα and 180-kd topoIIα. These two enzymes share considerable homology (72%) but are products of different genes located in chromosomes 17q21-q22 and 3p, respectively. The functions as well as the expression of these two genes are different. Whereas topoIIα expression is cell cycle-dependent, the β-isoform shows no cell cycle-phase dependency. The most abundant expression of topoIIα takes place at the G2/M-phase of the cell cycle and declines to minimum at the end of mitosis. The exact function of topoIIα is still largely unknown.

TopoIIα has raised considerable clinical interest since it is a molecular target for many antineoplastic and antimicrobial drugs. Among the cytotoxic drugs acting on inhibiting topoII are some of the most important anticancer drugs such as anthracyclines (e.g., doxorubicin, epirubicin, daunorubicin, idarubicin), epipodophyllotoxins (e.g., etoposide, teniposide), actinomycin and mitoxantrone. Although these anticancer drugs share no structural homology, they all act by trapping topoIIα in a covalently bound reversible complex with DNA, termed the ‘cleavable complex’. The stabilization of cleavable complexes prevents the religation of the double-stranded breaks. This converts topoIIα into a physiological toxin and introduces high levels of permanent double-stranded breaks that are ultimately detected by cell cycle checkpoint and culminate in cell death by apoptosis.

It has been shown in vitro that sensitivity to topoII-inhibitors correlates with the expression level of topoIIα in cancer cells. Cells with low nuclear concentrations of topoIIα protein form fewer topoII-mediated DNA strand breaks and are thus less sensitive to topoII-directed drugs than cells containing high amounts of topoIIα. This relationship was first established by comparing the chemosensitivity of different cell lines to their expression of topoII but more recently the relationship has been confirmed with more specific methods. These studies have shown that sensitive cell lines can be made resistant by transfection of either anti sense topoIIα mRNA or mutant topoIIα cDNA. The transfection of exogenous, wild-type topoIIα mRNA, in turn, reverses primary resistance to topoII-inhibitors into sensitivity.

D. Detection of TopoIIα

Detection of the amplification of the topoisomerase IIα (topoIIα) gene may be determined, for example, by employing in situ hybridization (e.g., FISH or CISH, See, Examples below). Probes for topoIIα may be obtained, for example, by screening a P1-library, and confirming the identity of the probe by performing PCR with topoIIα specific primers (See, Examples 1 and 10). BAC or PAC clones may also be used for TopoIIα probe preparation. In preferred embodiments, a TopoIIα probe that is capable of specifically detecting TopoIIα gene sequence (without falsely detecting HER2/neu) are employed. It should be noted that the TopoIIα probes briefly sold by Vysis (Downers Grove, Ill.), were unable to accurately discriminate between TopoIIα and HER2/neu. However, the present invention provides such specific probes (e.g., the Exemplary probe described in Example 10, and commercially available from Zymed Laboratories).

E. HER2 and HER-2/neu

The HER-2/neu oncogene (also known as erbB-2) encodes a 185-kDa transmembrane glycoprotein (HER2), which is a member of the family of epidermal growth factor (EGF) receptor tyrosine kinases (RTK). The HER-2 family of RTKs has four members: HER-1, HER-2, HER-3, and HER-4. The RTKs are cell-surface enzymes consisting of a single transmembrane domain separating an intracellular kinase domain from an extracellular ligand-binding domain. Ligand binding to the extracellular domain induces the formation of receptor dimers (homo- or preferentially hetero-), which are essential for activation of the intrinsic tyrosine kinase activity. This subsequently leads to a recruitment of target proteins, that initiate a complex signaling cascade.

Although a large number of putative candidate ligands (EGF, heparin binding EGF-like growth factor, transforming growth factor-\acute{α}, amphiregulin, betacellulin, epiregulin and a large family of different neuregulins among others) have been postulated to bind HER-2, none of these peptides binds HER-2 with high affinity. However, EGF-like ligands are bivalent. Thus, they are capable of binding their receptors at two different sites; namely high affinity as well as low affinity binding sites. Although HER-2 is not a high affinity receptor for any of the ligands shown to bind ErbBs, it is the preferred low affinity co-receptor for EGF-like ligands. Therefore, it emerges as the preferred dimer-mate for the three other ErbBs, once these primary receptors are occupied by their ligands. Thus, at least 20 growth factors can utilize HER-2 related signaling pathways.

HER-2 is vital in the induction of growth signal by the ligand occupied ErbBs, because in the presence of HER-2: 1) it is the preferred heterodimerization partner for all ligand-binding ErbB RTKs and 2) HER-2-containing heterodimers are also characterized by extremely high growth factor-induced signaling potency and mitogenesis. The high signaling potency of HER-2 containing heterodimers, in turn, is attributed to several specific features: 1) HER-2 reduces the rate of ligand dissociation from its high affinity receptor; 2) HER-2 induces lateral signaling by recruiting and activating other (unoccupied) ErbB receptors; and 3) HER-2 efficiently signals through protein kinases (such as MAP and Jun N-terminal), which are especially potent activators of mitosis. In addition, HER-2-containing receptor dimers are recycled from endosomes back to the cell surface instead of being degraded by lysosomes. Thus, these dimers may be overrepresented at the cell surface.

Due to these features, the HER-2 receptor has an oncogenic potential that may be activated through multiple genetic mechanisms including point mutations, truncation of the protein, and the amplification of the non-mutated proto-oncogene. However, gene amplification is by far the most common mechanism for the activation of the oncogenic potential of HER-2. The amplification of HER-2/neu happens in approximately 20 to 35% of invasive breast cancers and results in overexpression of the protein. Thus, the amplification of HER-2/neu increases the likelihood of HER-2 to form heterodimeric complexes with the other ErbBs. This, in turn, indicates that several dozen potentful ligands can take advantage of HER-2 dependent signaling pathways leading to the oncogenic activation of cells.

The association of HER-2/neu and the prognosis for breast cancer patients has been extensively studied (e.g., Ravdin and Chamness, Gene, 159:19-27, [1995]). Unfortunately, amplification of HER-2/neu has been found to be associated with poor clinical outcome. However, whether HER-2/neu is an independent prognostic factor is still controversial because both supportive and non-supportive results have been published (e.g., Ravdin and Chamness, supra).

The most common activation mechanism for HER-2/neu is by the amplification of the gene at 17q12-q21. The extra copies of HER-2/neu oncogene are deposited in cancer cells as extrachromosomal double minute chromosomes or within the chromosomes in homogeneously staining regions.

The predictive value of HER-2/neu has also been studied, although not as extensively as its prognostic value, in conjunction with both adjuvant chemotherapy and in chemotherapy for advanced breast cancer (e.g., McNeil, C., J. Natl. Cancer Inst., 91:100, [1999]). HER-2/neu appears to be a predictor for poor clinical outcome in adjuvant chemotherapy by conventional cyclophosphamide-methotrexate-fluorouracil-combination. The relationship of amplified HER-2/neu and topoII-inhibitor chemotherapy in breast cancer is more controversial. Most studies have linked amplified HER-2/neu to chemoresistance to topoII-inhibitors (See, e.g., Tetu et al., Mod. Pathol., 11:823 [1998]), but there are also clinical trials reporting either no association (See, e.g., Clahsen et al., J. Clin. Oncol., 16:470 [1998]), or even tendency for higher response rates among HER-2/neu-amplified breast tumors (See, e.g., Thor et al., J. Natl. Cancer Inst., 90:1346 [1998]). The results presented in Example 5 below support the conclusion that HER-2/neu amplification is not associated with clinical response to topoisomerase II inhibitors.

F. Detection of HER-2 and HER-2/neu

As noted above, HER-2/neu oncogene amplification and its concomitant protein overexpression are currently implicated as an important prognostic biomarker in breast carcinoma, and may also be a useful determinant of response to hormonal or cytotoxic chemotherapy. The clinical importance of HER-2/neu diagnostics has become even more significant with the increasing use of the new anti-cancer drug trastuzumab (HERCEPTIN, a humanized monoclonal antibody against the extracellular part of the HER-2/neu protein product). However, trastuzumab therapy is effective only in patients whose tumors contain amplification and/or overexpression of HER-2 (Shak. S., Semin. Oncol., 6:71 [1999]). Thus, HER-2 assays are now becoming an important part of breast cancer diagnostics, in parallel with assays of hormone receptors and tumor proliferation rate.

The earliest studies of HER-2 used Southern and Western blotting for detection of HER-2/neu gene amplification and HER-2 protein overexpression. However, these methods are not well-suited for routine diagnostics and have been replaced by immunohistochemistry and fluorescence in situ hybridization (FISH). In addition, a vast majority of HER-2 studies have been done using immunohistochemistry (IHC), which detects the HER-2 protein overexpression on the cell membrane. Without HER-2/neu oncogene amplification, the protein expression is generally low and undetectable by IHC. However, IHC is subject to a number of technical artifacts and sensitivity differences between different antibodies and tissue pretreatments. Standardized reagent kits have recently been introduced (e.g., HERCEP-TEST, DAKO Corp.), but mixed results have been reported from their methodological comparisons (Jiminez et al., Mod. Pathol., 13:37 [2000]). Other HER-2 commercially available antibodies include two monoclonal antibodies from Novocastra Laboratories, clone CB-11 and NCLB12, and the antibodies described above in section II.

Fluorescent in situ hybridization (FISH) quantifies the number of gene copies in the cancer cell nucleus. Since the initial experiments to detect HER-2/neu amplification by FISH, a number of reports have verified its accuracy both in freshly frozen and paraffin-embedded tumor material (Mitchell, M. S., Semin. Oncol., 26:108 [1999]). FISH is generally performed using either single-color (HER-2/neu probe only) or dual-color hybridization (using HER-2/neu and control probes (e.g., chromosome 17 centromere probes simultaneously), with the latter method making it easier to distinguish true HER-2/neu amplification from chromosomal aneuploidy. FISH using entire cells (e.g., cultured cells, pulverized tissue, or imprint touch specimens from tumors) is considered straightforward, but the use of tissue sections complicates the quantitative nature of FISH due to nuclear truncation (i.e., due to the slicing of the tissues during their preparation for staining). Commercially available FISH probes include Zymed's SPOT-LIGHT HER-2/neu probe (Zymed Laboratories, San Francisco, Calif.), and Vysis's LSI HER-2/neu SpectrumOrange probe (Vysis, Downer's Grove, Ill.).

The main difficulty in adopting FISH for clinical diagnostic use is the requirement for fluorescence microscopy. Evaluation of FISH samples generally requires a modern epifluorescence microscope equipped with high-quality 60× and 100× oil immersion objectives and multi-bandpass fluorescence filters. Moreover, because the fluorescence signals fade within a few weeks, the hybridization results usually must be recorded with expensive CCD cameras.

One aspect of the present invention circumvents many of these problems by providing methods and compositions for detecting HER-2/neu that are rapid and do not require the use of fluorescence microscopy. In particular, the present invention provides Chromogenic In Situ Hybridization (CISH) HER-2/neu detection probes and methods (See, Examples 7, 8, and 9) that allow enzymatic detection of HER-2/neu. As described in these examples, the present invention provides HER-2/neu probe libraries capable of detection by bright field microscopy. Such probes and detection reagents are commercially available from Zymed Inc. (South San Francisco, Calif.). Another advantage of the HER-2/neu probe is the ability to perform CISH and histopathology simultaneously on the same tissue sample (See, Example 9). A further advantage of using CISH is the ability to view CISH signal and cell morphology at the same time.

G. HER-2/neu-TopoIIα Relationship

The relationship between HER-2/neu and topoIIα amplification has been previously studied. Indeed, topoIIα has been found to be amplified in breast tumors with HER-2/neu amplification [e.g., Smith et al., Oncogene, 8:933 (1993)]. As TopoIIα and HER-2/neu are located so close to each other on chromosome 17, that a simple molecular mechanism for this phenomenon previously hypothesized involves amplification of the chromosomal segment bearing both genes [Murphy et al., Int. J. Cancer, 64:18-26 (1996), Hoare et al., Br. J. Cancer, 75:275 (1997)]. This should lead to similar gene copy numbers for HER-2/neu and topoIIα. However, during the development of the present invention, as detailed in Example 3, imbalanced copy numbers for HER-2/neu and topoIIα were found by employing fiber FISH analysis. As discussed in Example 3, the presence of two separate amplicons for closely situated genes such as HER-2/neu and topoIIα was unexpected.

The relationship between HER-2/neu and topoIIα amplification and the response of breast cancer cell lines to topoisomerase inhibitors has also been previously been studied. For example, one group reported that a breast cancer cell line with amplification of both HER-2/neu and topoIIα was the most sensitive to m-AMSA and mitoxantrone. [Smith et al., supra]. Subsequent to the breast cancer cell line work, the effect of topoisomerase inhibitors on primary breast cancer cells was evaluated in primary breast cancer cells determined to have amplified HER-2/neu and topoIIα [Jarvinen, et al., British Journal of Cancer, 77(12):2267 (1998)]. However, instead of confirming the results previously reported for breast cancer cell lines, the primary breast cancer cells with amplification of both HER-2/neu and topoIIα were not found to exhibit a positive response to topoisomerase inhibitors. Thus, the art would predict that the present invention would not work. Nonetheless, the surprising results obtained during the development of the present invention indicates that the methods described herein do work. In this regard, the results presented in the Examples below were unexpected.

H. HER-2/neu-TopoIIα Status as Diagnostic Marker

The present invention provides diagnostic markers for cancer (e.g., breast cancer). In particular, the present invention provide methods for determining whether a candidate subject is suitable for topoisomerase II inhibitor treatment or anti-HER2 immunotherapy by detecting copy number amplification of both HER-2/neu and topoIIα. In some embodiments, the present invention provides methods for identifying a candidate for topoisomerase II inhibitor treatment by providing a candidate subject suspected of having breast cancer cells and detecting a copy number for both HER-2/neu and topoIIα in the breast cancer cells. In this regard, the method allows identification of the candidate subject as suitable for treatment with a topoisomerase II inhibitor by demonstrating amplification of the copy number for both the HER-2/neu and the topoIIα. In some embodiments, the candidate subject has breast cancer cells comprising an amplified copy number for HER-2/neu. (e.g., HER-2/neu amplification was already determined). In other embodiments, the candidate subject is determined to have HER2 gene amplification, but not topoIIα gene amplification. These subjects, in some embodiments, are administered anti-HER2 immunotherapy (e.g. HERCEPTIN) without concomitant topoisomerase II inhibitors (e.g. such that the elevated risk of cardiovascular side effects from combined HER2 immunotherapy and anthracycline administration is avoided). In other words, subjects found to have an amplified HER2 gene copy number, but not an amplified topoIIα gene copy number, are identified as suitable for topoisomerase II inhibitor-free (e.g. anthracycline-free) anti-HER2 antibody therapy (i.e. the subject is not administered both topoisomerase II inhibitors and anti-HER2 antibodies around the same time in order to avoid, for example, cardiac problems found in patients given the combination therapy).

In certain embodiments, the detecting is performed with HER-2/neu and topoIIα specific probes (e.g., fluorescent in situ hybridization, chromogenic in-situ hybridization, or both FISH and CISH). While not limiting the present invention to any particular mechanism, and not necessary to the successful practice of the present invention, it is believed that detecting the nucleic acid of topoIIα instead of the expressed protein product (e.g., by immunohistochemistry) allows amplification of both HER-2/neu and topoIIα to serve as a diagnostic marker for breast cancer cells susceptible to treatment with topoisomerase II inhibitors. In particular, as demonstrated in Example 4, there is a lack of correlation between topoIIα gene status and immunohistochemical (IHC) detection. Consequently, assessment of topoIIα gene expression using IHC detection fails to yield a relationship between amplification of both topoIIα and HER-2/neu in regard to predicting the response of primary breast cancer cells to topoisomerase II inhibitors. Thus, the present invention provides a breast cancer marker for response to anthracycline based therapy by detecting copy number for both HER-2/neu (e.g., employing IHC or nucleic acid probes) and topoIIα (e.g., employing nucleic acid probes). As such, the present invention provides improved methods for identifying breast cancer patients suitable for treatment with topoisomerase II inhibitors, as well as patients that should not receive topoisomerase II inhibitors (e.g., anthracycline). In this regard, patients that are candidates for anti-HER2 immunotherapy (e.g. have increased HER2 expression and/or increased HER2 gene amplification), but do not have amplification of the topoIIα gene (e.g. unlikely to benefit from anthracycline administration) may be administered anti-HER2 antibodies without risking the side effects of anthracylines, and the increased risk of cardiovascular problems, by not administering anthracyclines to these patients.

Importantly, the present invention allows assessment of patients found to have HER-2/neu amplification (i.e., an indicator for HERCEPTIN treatment). Indeed, testing to determine whether anthracycline treatment is appropriate (amplification of both HER-2/neu and topoIIα) or if HERCEPTIN treatment is appropriate (only HER-2/neu amplification). This capability is of particular importance in view of the human trials that have identified serious risks associated with co-administering both anthracyclines and HERCEPTIN.

IV. Combined CISH and IHC

In some embodiments, the present invention provides methods, compositions, and kits for combined chromogenic in-situ hybridization (CISH) and immunohistochemistry (IHC). The combined CISH and IHC methods of the present invention allow for comprehensive and valuable information regarding a tissue sample (and patient status) to be determined. In certain embodiments, CISH and IHC are performed on the same tissue sample (e.g. on same part of tissue sample or adjacent sections). In other embodiments, CISH and IHC are performed on different tissue samples, but the tissue samples are from the same biological sample (e.g. from the same breast cancer biopsy sample or gastric cancer tissue sample). In particular embodiments, CISH and IHC are performed simultaneously or nearly simultaneously (e.g. on the same tissue sample or separate tissue samples from the same biological sample). In preferred embodiments, CISH is performed first and IHC is performed second (See, e.g. Example 9).

In other preferred embodiments, a biological sample is tested by both CISH and IHC (e.g. in order to confirm the presence or absence of HER2 gene amplification and HER2 over expression). In certain embodiments, a tissue sample (e.g. breast cancer biopsy sample or gastric cancer tissue sample) is tested for HER2 gene amplification by CISH and HER2 overexpression by IHC prior to administering (or recommending) anti-HER2 antibody therapy (e.g. HERCEPTIN therapy).

In certain embodiments, CISH and IHC are performed on the same tissue section. For example, the first few steps of CISH may first be performed on the tissue section (e.g. pretreatment, hybridization, and wash). Then, the second party of CISH and IHC may be performed at, or about, the same time. For example, different antibodies may be used. For example, the CISH antibodies may be raised in a first species (e.g. mouse), and the IHC antibodies by be raised in a second species (e.g. rabbit). Also, the detection enzymes used for CISH and IHC may be different, such that the signals can be evaluated individually. For example, the CISH antibodies may be conjugated to HRP, while the IHC antibodies may be conjugated to AP. In this regard, the CISH methods may use a substrate such as DAB, while the IHC methods may use a different substrate such as FAST RED.

V. Subtracted Probes

The present invention provides subtracted probes (e.g. subtracted probe libraries) useful for in-situ hybridization methods (e.g. FISH, CISH, etc.). In certain embodiments, the probe libraries are substantially free of repeating sequences (e.g. ALU and LINE elements). For example, in some embodiments, the probe libraries have at least 90% of the repeat sequences removed (e.g. the probe libraries comprise 10% or less of repeat sequences). In other embodiments, the probe libraries have at least 95% of the repeat sequences removed (e.g. the probe libraries comprise 5% or less of repeat sequences). In preferred embodiments, the CISH methods, kits, systems and compositions of the present invention are practiced with subtracted probe libraries. Importantly, in certain embodiments, the use of subtracted probes allows a clear signal to be obtained when performing CISH methods (e.g. on cancer biopsy samples).

In some embodiments, the subtracted probe libraries are prepared substantially as described in WO0026415 to Fletcher et al., or as described in U.S. Pat. No. 6,569,621 to Craig and Cremer, both of which are herein incorporated by reference. In certain embodiments, the subtracted probe libraries are performed according to the following procedure. First, clones (e.g. YACs, BACs, or PACs) are chosen that span a gene of interest, or that are on either side of a gene of interest in the case of probe pair libraries. Next, the clone is broken down (e.g. by sonication) into smaller pieces (e.g. 0.1-8 kb fragments) to form the probe library. Then, in certain embodiments, adapters are ligated on the ends of the fragments (or in some embodiments, adapters are not employed). Next, PCR is performed on the fragments (e.g. using primers specific for the adapters, or random primers). Next, gel size and purification is performed to select a library of fragments in a given range (e.g. 0.5-4 kb). After that, a subtraction step is performed with labeled repeat (driver) nucleic acid (e.g. biotin labeled COT-1 DNA). The labeled driver nucleic acid is then allowed to hybridize with the library of fragments (tracer nucleic acid). The driver nucleic acid will hybridize to fragments containing complementary repeat sequences (and generally not hybridize to fragments that do not contain these repeat fragments). The mixture is then exposed to a solid support (e.g. beads) conjugated to a second label specific for the label on the driver nucleic acid. In this regard, the driver nucleic acid and the fragments containing repeat sequences hybridized to the driver nucleic acid, are removed from the reaction solution. As a result, the remaining library of fragments has most of the repeat sequences physically subtracted out. The remaining subtracted library may be subjected to further rounds of PCR (e.g. 3 additional rounds of PCR), and then labeled with a desired label (e.g. digoxigenin). The subtracted probe library may then be used in in-situ hybridization procedures, and generally, does not require a blocking step (e.g. the probes dont have to be blocked with repeat sequences, and the tissue sample also does not have to be blocked with repeat sequences).

In some embodiments, the present invention provides a HER2 gene probe library (See, e.g., Example 7). In preferred embodiments, this probe library comprises 90%, preferably 95% repeat free fragments. In some embodiments, the HER2 gene library is specific for the HER2 gene, and is capable of detecting HER2 gene amplification. In particular embodiments, the present invention provides a topoIIα gene probe library (See, e.g., Example 10). In preferred embodiments, this probe library comprises 90%, preferably 95% repeat free fragments. In other embodiments, the topoIIα gene library is specific for the topoIIα gene (e.g. does not falsely detect HER2 gene amplification), and is capable of detection TopoIIα gene amplification. In additional embodiments, the present invention provides an EGFR probe library (See, e.g., Example 13). In preferred embodiments, this probe library comprises 90%, preferably 95% repeat free fragments. In some embodiments, the EGFR probe library is specific for the EGFR gene, and is capable of detecting EGFR gene amplification (e.g. by CISH or FISH). In other embodiments, the present invention provides an N-MYC probe library (See, e.g., Example 15). In certain embodiments, the N-MYC probe library comprises 90%, preferably 95% repeat free fragments. In some embodiments, the N-MYC probe library is specific for the N-MYC gene, and is capable of detecting N-MYC gene amplification.

In some embodiments, the present invention provides subtracted library probe pairs for detecting gene translocations. In certain embodiments, the probe pairs are “split-apart” probe pairs and are configured hybridize to the centromeric and telomeric regions out side of the breakpoints of targeted genes (e.g. ABL and SYT genes). Additional details on ABL split apart probe pairs, and disease detection are provided below in section VI. The breakpoints are located between the gap of the centromeric and telomeric probes such that all (or most) translocations are detected. Split-apart probe pairs, in a normal cell without translocation, show two pairs of dots (e.g. dot has two dots in juxtaposition). The two dots in each pair, for example, shows two different colors in CISH. Also with split-apart probes, a cell with translocation also shows 2 pairs of dots. One pair has two dots in juxtaposition representing the normal chromosome in the cell, the other pair dots are separated representing the translocated chromosome in the cell.

VI. ABL Probe Pairs and Detecting BCR-ABL Translocations

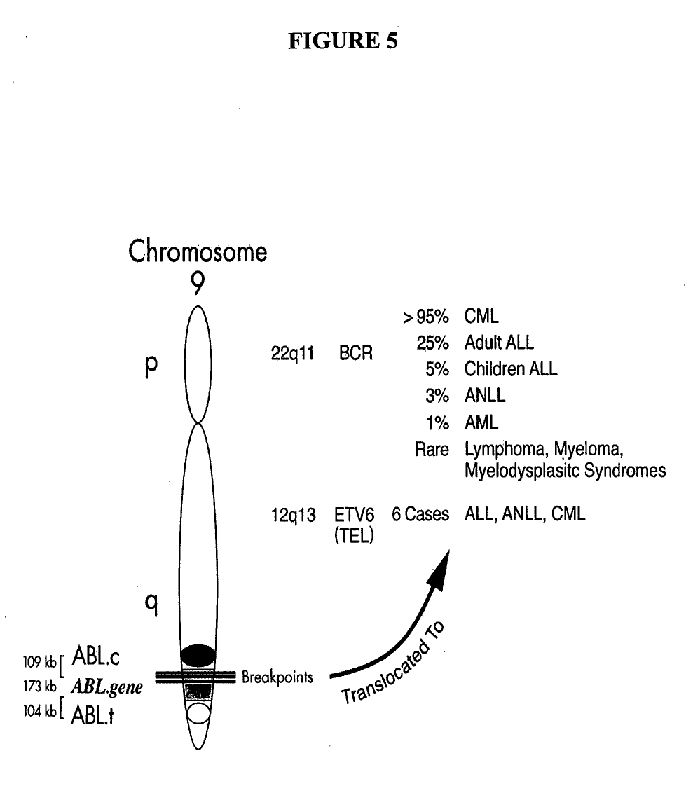

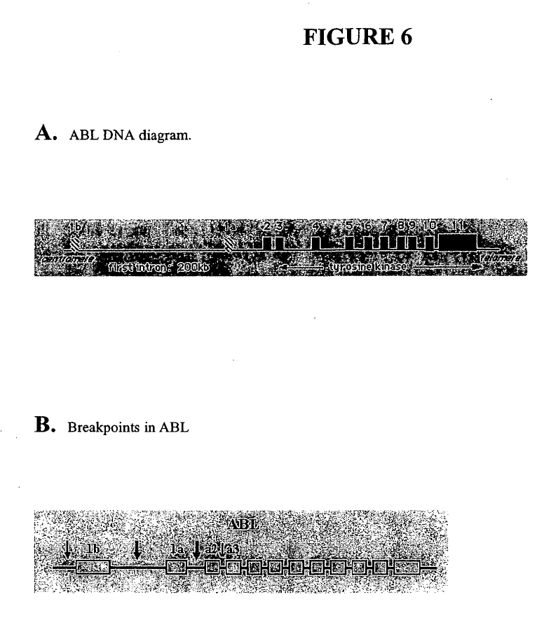

As mentioned above, the present invention provides ABL probe pairs that may be used, for example, to detected BCR-ABL translocations. One example of how to prepare the split-apart ABL probe pair is provided in Example 14. The labeled ABL probe pair can detect BCR-ABL translocation by ISH (e.g. CISH or FISH) on cells from, for example, chronic myeloid leukemia (CML). The staining pattern in these tumor cells is distinctively different from that in normal cells. Translocations involved in the ABL gene (FIG. 5) are found, for example, in CML, acute lymphoblastic leukemia (ALL), acute non-lymphocytic leukemia (ANLL), and acute myeloid leukemia (AML). Breakpoints in the ABL gene is variable over a region of about 200 kb (FIG. 6). The ABL translocation probe pairs of the present invention are, in some embodiments, able to detect all types of the ABL translocations reported so far. Examples of ABL translocations the ABL probe pairs of the present invention are able to detect are described below.

i. ABL (Abelson Murine Leukemia Oncogene) Gene and Protein

The ABL protooncogene spans about 230 kb of genomic DNA, has 12 exons (FIG. 6), and expressed as either 6 or 7 kb mRNA transcript, with alternatively spliced first exons, exon 1b and 1a, respectively, spliced to the common exons 2-11. Exon 1 b is approximately 200 kb 5-prime of exon 1a (FIG. 6). The very long intron is a target for translocation in leukemia (see, Bernards et al., 1987, Molec. Cell. Biol. 7:3231-6, herein incorporated by reference). Breakpoints in the ABL gene are variable over a region of about 200 kb (FIG. 6), often occurring between the two alternative exons 1b and 1a, sometimes 5′ of 1b or 3′ of 1a. The breakpoint in the intron between exons a2 and a3 of the ABL gene is rarely found.

The ABL gene maps to chromosome band 9q34.1. ABL as well as BCR gene regions have extremely high density, 39.4% and 38.83% respectively, of Alu homologous regions (See, Chissoe et. al, 1995, Genomics, 27:67-82). The 145 kD ABL protein is homologous to the tyrosine kinase (SH1) and regions 2 and 3 (SH2, SH3) of SRC (the chicken Rous sarcoma virus). ABL protein, like SRC, is a non-receptor tyrosine kinase and it has weak enzymatic activity. ABL protein is ubiquitously expressed and expression is located mainly in the nucleus to bind DNA but can migrate into the cytoplasm. Both ABL and transforming ABL proteins inhibit cell entry into S phase by a mechanism that requires nuclear localization and is p53 and pRb dependent (See, Welch and Wang, 1993, Cell, 75:779-790, herein incorporated by reference). Interaction of ABL protein with pRb can promote E2F1-driven transcription, for example, of Myc. Alterations of ABL by chromosomal rearrangement or viral transduction lead to malignant transformation, as in CML. Activity of ABL protein is negatively regulated by its SH3 domain, and deletion of the SH3 domain turns ABL into an oncogene.

ii. BCR (Breakpoint Cluster Region) Gene and Protein

The BCR gene spans about 130 kb of genomic DNA, has 23 exons and maps to chromosome band 22q11.2. It is proximal to EWS and NF2 genes, both in 22q12. Three breakpoint cluster regions have been characterized to date: major (M-bcr), minor (m-bcr) and micro (m-bcr). Breakpoint in M-bcr, a cluster of 5.8 kb, is between exons 12 and 16, also called b1 to b5 of M-bcr. Breakpoint in m-bcr is in a 35 kb region between exons 1 and 2. Breakpoint in m-bcr is in intron 19. The 160 kD BCR protein has serine/threonine protein kinase activity. BCR protein is widely expressed in many types of human haematopoietic and non-haematopoietic cells and cell lines.

iii. CML

CML is a malignant clonal disorder of pluripotent hematopoietic stem cells resulting in an increase of myeloid, erythoid, and platelet cells in peripheral blood, and myeloid hyperplasia in the bone marrow. CML is an insidious cancer. It starts out as a genetic flaw spurring overproduction of platelets and white blood cells. Early symptoms are surprisingly few and mild, such as chills and malaise. The typical symptoms of the disease are splenomegaly, fatigue, anorexia and weight loss. Over a few years, the genetically defective cells slowly accumulate more mutations. Eventually the proliferating malignant cells crowd out good cells and the body can no longer fight infection. Though the median age of CML patients is 53 years, the disease also occurs in children. The annual incidence of CML is 10/106 (from 1/106 in childhood to 30/106 after 60 yrs). The disease progresses from the benign chronic phase, usually through an accelerated phase, to the fatal blast crisis within 3-4 years. In contrast to the chronic phase, leukocytes in the blast crisis fail to mature and they resemble myeoblasts or lymphoblasts in patients with acute leukemias. This progression is likely related to the genetic instability induced by BCR-ABL, and is commonly associated with the acquisition of additional, and frequently characteristic, genetic changes. The Ph chromosome and BCR-ABL fusion, however, persist through all phases.

Approximately 4500 new cases of CML occur in the United States each year. The only cure for CML which afflicts 25,000 adults in the U.S. is a bone marrow transplant. But 80% of patients can't find a suitable donor or are too old to risk a transplant. The procedure costs about $150,000 and kills up to 25% of those who undergo it. Drug therapy with alpha-interferon only slows the disease and side effects are so severe that many can't bear it. Fortunately, a newly developed drug has been developed by Novartis called GLEEVECA (ST1571). GLEEVECA is a small molecule inhibitor of ABL, the first leukemia drug designed to attack the molecular machinery that drives the disease. In one experiment using ST1571, 56% of 290 patients who had given up on other therapies enjoyed a partial or complete elimination of cancer cells from their bone marrow. For some patients, traces of cancer can no longer be detected even with exquisitely sensitive DNA probes. Although ST1571 has produced complete responses in CML, resistance in some patients highlights the need for other drugs. According to research by investigators at Memorial Sloan-Kettering Cancer Center and Rockefeller University, a new drug called PD17 demonstrated significantly greater potency than ST1571 against BCR/ABL containing cell lines and CML patient's cells. PD17 is a member of a class of tyrosine kinase inhibitors originally synthesized by Parke Davis and shown to be potent inhibitors of src family kinases. In addition, another experiment showed that combination of ST1571 with adaphostin induced more cytotoxicity in vitro than either agent alone (See, Mow B M F, et al., Blood, 99:664-71, 2001, herein incorporated by reference).

iv. BCR-ABL Translocation

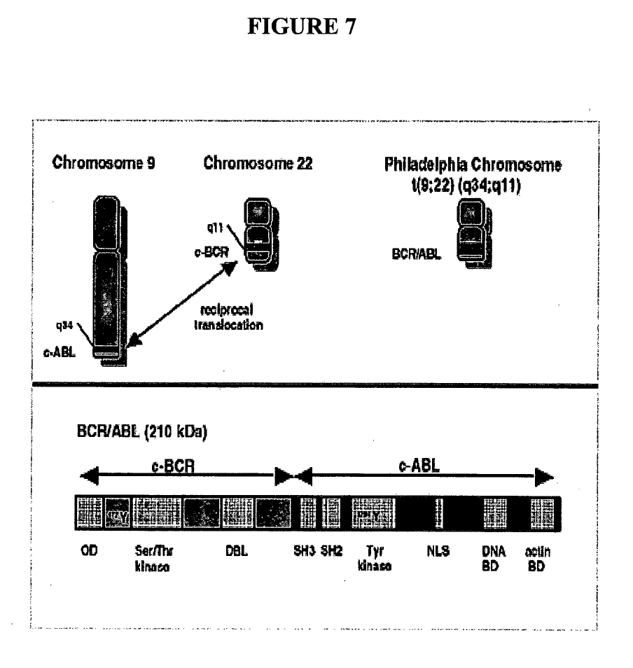

CML was the first malignancy shown to have an acquired and specific genetic abnormality, with the identification of the Philadelphia (Ph) chromosome in 1960, an abnormally shortened chromosome 22. It was demonstrated later (1973) that the Ph chromosome resulted from a reciprocal t(9;22) translocation (FIG. 7). The molecular correlates of this translocation were first identified in 1983, with subsequent recognition of the fusion of two distinct genes, BCR and ABL (See, De Klein et al., Nature, 330:765-767, 1982, herein incorporated by reference), resulting in the head-to-tail fusion of the BCR and ABL genes (See, Chissoe et. al, Genomics, 27:67-82, 1995, herein incorporated by reference) (FIG. 7).

The BCR/ABL fusion protein greatly increases ABL's tyrosine kinase activity. Its ability to cause CML was demonstrated by retrovirally-transfecting BCR-ABL into mice in 1990, which led to the induction of a CML-like syndrome (See, Scott et al., PNAS, 88:6506-6510, 1991, herein incorporated by reference). While the precise subcellular pathways through which these ultimate biological consequences are attained remain to be definitively dissected, the fact that BCR-ABL is indeed the cause of CML appears clear now, based on the clinical response to targeted tyrosine kinase (TK) inhibition with the drug imatinib mesylate (formerly known as ST1571), trade-named GLEEVACA.

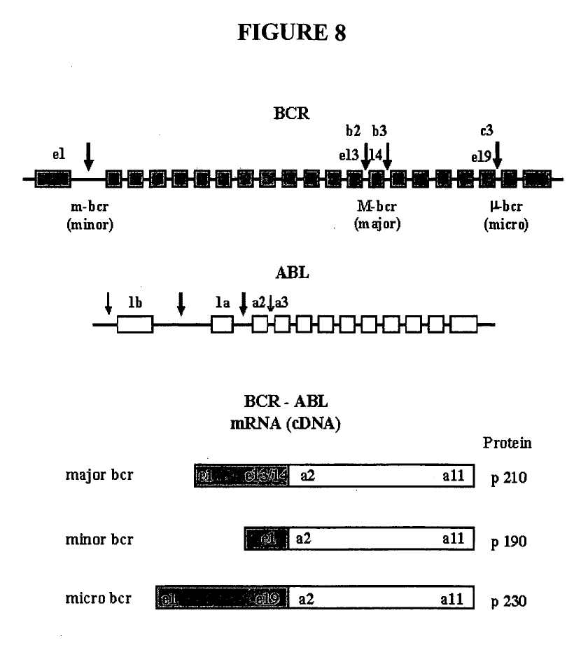

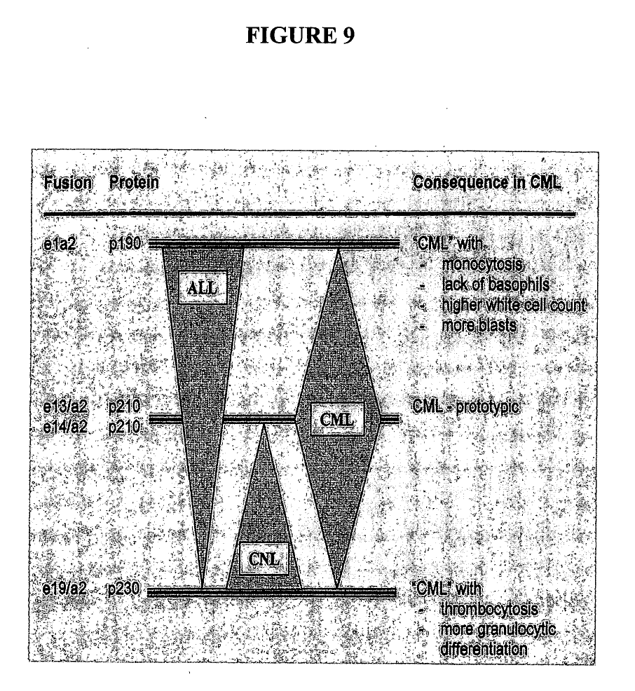

The BCR-ABL hybrid gene, the main product of the t(9;22)(q34;q11) translocation, is found in >95% of CML patients. The BCR-ABL translocation is not exclusive to CML and it is also present in a minority of some other oncohematological diseases, e.g. in about 25% of adult and 5% of children ALL, 1% AML, and very rarely in lymphoma, myeloma or myelodysplastic syndromes. The presence of the BCR/ABL gene does not generally have a diagnostic significance in these diseases but, at least in ALL, it has a prognostic importance, i.e. it is a negative prognostic factor. The fusion protein encoded by BCR-ABL varies in size, depending on the breakpoint in the BCR gene. Three breakpoint cluster regions (FIG. 8) have been characterized to date: major (M-bcr), minor (m-bcr) and micro (m-bcr) (Melo J V, Baillieres, Clin. Haematol. 10:203-222, 1997, herein incorporated by reference). The overwhelming majority of CML patients have a p210 BCR-ABL gene (M-bcr), whose mRNA transcripts have a b3a2 and/or a b2a2 junction (FIG. 9). The smallest of the fusion protein, p190BCR-ABL (m-bcr breakpoint), is principally associated with Ph-positive-ALL (Fainstein et al., Nature, 330:386-388, 1987, herein incorporated by reference). CML resulting from p230 BCR-ABL gene (m-bcr breakpoint) is also rare. The micro breakpoint has been associated mainly with a mild form of CML, defined as Philadelphia chromosome-positive neutrophilic-chronic myeloid leukemia (Ph-positive CML-N). Exceptional CML cases have been described with BCR breakpoints outside the three defined cluster regions, or with unusual breakpoints in ABL resulting in BCR-ABL transcript with b2a3 or b3a3 junctions, or with aberrant fusion transcripts containing variable lengths of intronic sequence inserts (Melo, supra).

Approximately 5-10% of patients with CML have deletion of the 5′ region of ABL and the 3′ region of the BCR gene on 9q+chromosome. The deletions at 5′ region of ABL gene, in many cases, can span several megabases. The evidence suggests that these large deletions are associated with a poor prognosis of CML. ETV6-ABL translocation, t(9;12)(q34;q13) are reported in 6 cases of ALL, ANLL and CML (Andreasson P, et al., Genes Chromosome Cancer, 20:299-304, 1997; Hannemann J R, et al., Genes Chromosomes Cancer, 21:256-259, 1998, both of which are herein incorporated by reference). The breakpoints involved in EWS translocation in chromosome 22 is distal to that in translocation (8;22)-positive Burkitt lymphoma and that in translocation (9;22)-positive chronic myeloid leukemia.

iv. Split Apart ABL Probes

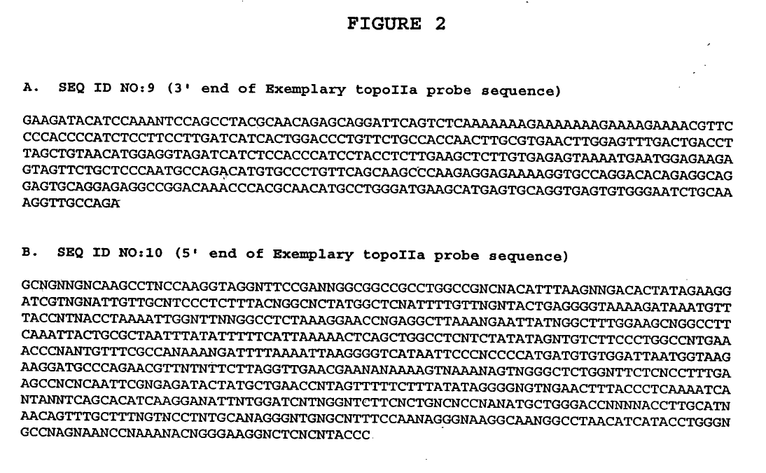

In some embodiments, the present invention provide split apart ABL probe pairs that are able to hybridize to both the centromeric and telomeric regions outside of the ABL gene. In preferred embodiments, the ABL probe pair comprises a probe set configured to hybridize to a region that is centromeric of the ABL gene, and a probe set that is configured to hybridize to a region that is telomeric of the ABL gene. Preferably, the probe sets comprise subtracted nucleic acid fragments (e.g. less than 90 or 95% or repeat sequences are present), and are detectably labeled. The ABL probe pairs of the present invention may be employed to detect ABL translocations (see above and FIG. 5) in order to diagnose a patient suspected of having this type of disease. Example 14 provides one example of ABL split apart probe pairs may be generated. In preferred embodiments, the ABL probe pair is configured such that all the breakpoints in the ABL gene are located between the gap of centromeric and telomeric probes. The ABL probe pair of the present invention may be used in, for example, in situ hybridization methods (e.g. CISH and FISH) in order to screen patient samples for ABL rearrangements (e.g. See, FIG. 2). The ABL probes of the present invention should also allow localization of previously uncharacterized translocation partners.

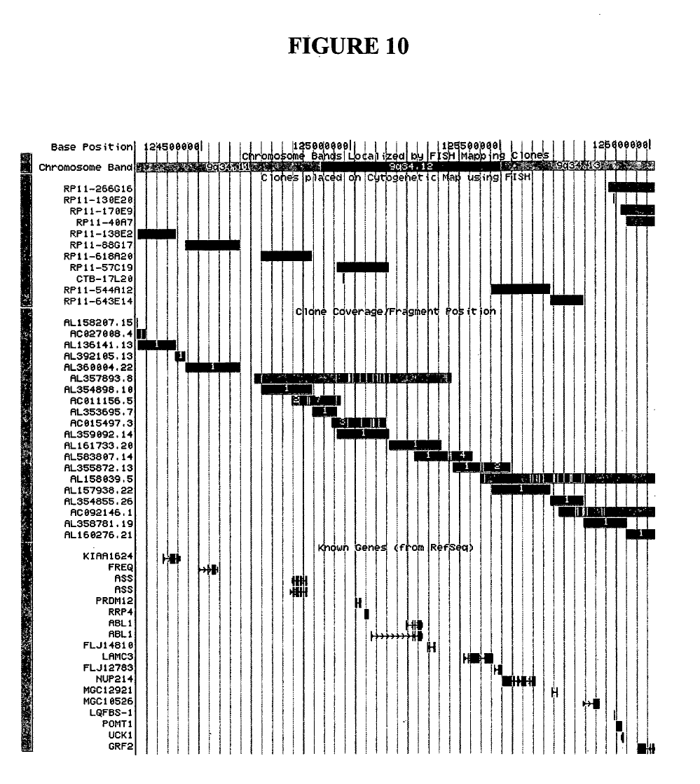

The ABL probe pairs may be generated, for example, as described in Example 14. Also, additional starting clones (e.g. BACs, YACs) selected using computer databases (e.g. human genome sequence information available on the internet) to select sequences on the telomeric and centromeric sides of the ABL gene. For example, FIG. 10 provides a printout of the UCSC genome browser for the ABL gene that may be employed to identify suitable clone sequences to generate the ABL split apart probe pair. Preferably, repeat sequences are removed from both probe sets (See, e.g. Example 14), such that cell samples do not need to be blocked prior to in situ hybridization. Also, in preferred embodiments, the centromeric and/or telomeric probe sets (e.g. comprising fragments 0.1 to 8 kb in length) have a combined hybridization length of at least 50 kb, preferably 100 kb, more preferably 200 kb, and most preferably at least 250 kb. In certain embodiments, the ABL.c (centromeric probe set) is approximately 250 kb in length (e.g. 200-300 kb), and the ABL.t (teleomeric probe set) is approximately 250 kb in length (e.g. 175-325 kb in length).

In certain embodiments, the ABL probe pair is provided in a kit. For example, in some embodiments, the kit comprises an ABL telomeric probe set, an ABL centromeric probe set, and instruction for employing the probe pair (e.g. to detect disease related to ABL rearrangement, such as those listed in FIG. 5). In further embodiments, the kits comprise reagents necessary for performing FISH or CISH.

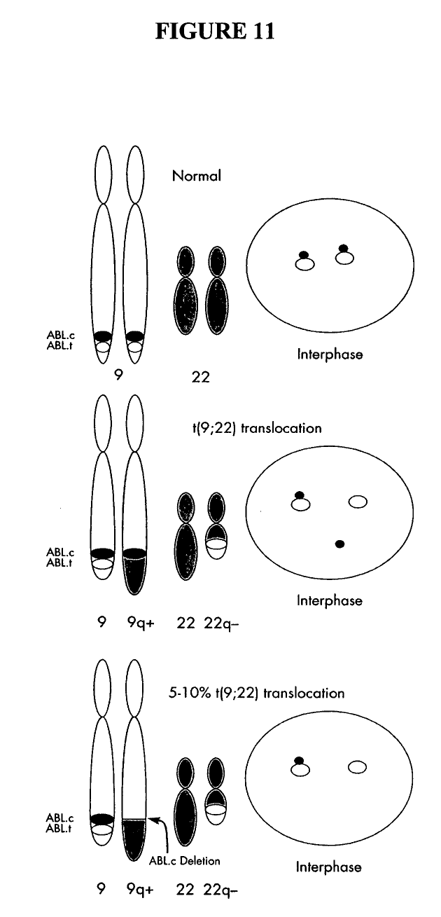

Interpreting the results of in situ hybridization on a cell sample (e.g. patient sample) may be performed as described above for the split apart probes of the present invention. For example, FIG. 11 shows graphically how results look for “normal” (probe pairs next to each other) and translocation (one probe pair split apart). FIG. 11 also shows the results that may be present in about 5-10% of CML cases that have a deletion that will, at least in some cases, result in loss of chromosomal material centromeric to the chromosome 9 breakpoint. ABL.c might be deleted in up to 5-10% of CML. The Zymed probe an advantage over the traditional bring-together probes to detect fusion genes, e.g. BCR/ABL probe from Vysis since the ABL split apart assay reveals both translocation and associated deletion.

In some embodiments, the patient sample (e.g. cell biopsy sample) is treated with the ABL probe pairs of the present invention, an ABL translocation is detected, and then the patient is identified as suitable for treatment with GLEEVACA, PD17, or other suitable treatment. In other embodiments, the patient sample is treated with the ABL probe pairs of the present invention, ABL translocation is detected, and then the patient is administered GLEEVACA, PD17, or other suitable treatment.

VII. MALT1 Probe Pair

In some embodiments, the present invention provide split apart MALT1 probe pairs that are able to hybridize to both the centromeric and telomeric regions outside of the MALT1 gene. In preferred embodiments, the MALT1 probe pair comprises a probe set configured to hybridize to a region that is centromeric of the MALT1 gene, and a probe set that is configured to hybridize to a region that is telomeric of the MALT1 gene. Preferably, the probe sets comprise subtracted nucleic acid fragments (e.g. less than 90 or 95% of repeat sequences are present), and are detectably labeled. The MALT1 probe pairs of the present invention may be employed to detect MALT1 translocations in order to diagnose a patient suspected of having, for example, MALT lymphomas. Example 17 provides one example of how MALT1 split apart probe pairs may be generated and employed. In preferred embodiments, the MALT1 probe pair is configured such that all the breakpoints in the MALT1 gene are located between the gap of centromeric and telomeric probes. The MALT1 probe pair of the present invention may be used in, for example, in situ hybridization methods (e.g. CISH and FISH) in order to screen patient samples for MALT1 rearrangements. In certain embodiments, the centromeric MALT1 subtracted probe library is approximately 500-575 kb in length (e.g. about 500 kb, about 530 kb, or about 550 kb). In other embodiments, the teleomeric MALT1 subtracted probe library is approximately 530-610 kb in length (e.g. about 540 kb, about 570 kb, or about 600 kb).

The MALT1 gene (Mucosa Associated Lymphoid Tissue gene) is located on chromosome 18q21 and is involved in t(11;18) and t(14;18) seen in MALT lymphomas. The MALT1 subtracted Probe Pair can be used in a screening mode to help identify and group patients with aggressive MALT lymphomas, and to further determine the course of treatment. t(11; 18) is a marker to group patients who will not respond to H pylori eradication (see, Liu et al, Gastron., 122:1286-94, 2002, herein incorporated by reference).

In 1983, Isaacson and Wright introduced MALT lymphoma to characterize primary low-grade gastric B-cell lymphoma (Isaacson and Wright, Cancer, 52:1420-6, 1983, herein incorporated by reference) and has since been extended to characterize other extranodal low-grade B-cell lymphomas of the salivary gland, lung, and thyroid, which account for approximately 5% of all non-Hodgkin's lymphomas (Dierlamm et al., Hematol. Oncol., 18:1-13, 2000). By the World Health Organization, these lymphomas have been classified and designated as ‘extranodal marginal zone B cell lymphoma of mucosa-associated lymphoid tissue’. Gastric MALT lymphoma is the most common and best characterized. To acquire gastric MALT lymphoma, Helicobacter pylori (H. pylori) must survive the gastric mucosa, which is protected by gastric acid and a thick layer of mucous, by secreting urease to neutralize the local pH. The infection leads to the accrual of lymphoid tissue that contains B-cell follicles resulting in lymphoid tissue with morphological features of MALT (Du et al., Lancet Oncol., 3:97-104, 2002). As an acquired reaction to H pylori infection, gastric MALT lymphoma arises from the mucosal lymphoid tissue. It is localized in the antrum or distal body of the stomach. Gastric MALT lymphoma can migrate to the small intestine, salivary gland, and splenic marginal zone. Transformation from MALT lymphoma to diffuse large B-cell lymphoma (DLBCL) has been seen in some cases.

In regard to MALT translocations, t(11;18)(q21;21) is the most frequent chromosomal aberration occurring in 30-40% MALT lymphomas. The two genes involved in this translocation are the mucosa-associated lymphoid tissue (MALT) gene, which is classified as a paracaspase, and the API2 gene, which is classified as an apoptosis inhibitor. The resulting API2-MALT1 fusion transcript is comprised of the N-terminal region of API2 and the C-terminal region of MALT, and is crucial in lymphocyte activation and survival. Liu et al found this translocation to be exclusive to gastric MALT lymphoma that did not respond to H pylori eradication (Liu et al., Lancet, 2001, 357:39-40, herein incorporated by reference).

This translocation is a reliable marker for aggressive gastric MALT lymphomas and aids in grouping patients who will not respond to H pylori eradication, therefore requiring chemotherapy or radiotherapy. t(1;14)(p22;q32) is believed to be exclusive to MALT lymphomas of the stomach and lungs occurring in less of than 5% of MALT lymphoma cases. This translocation involves BCL10 gene and the immunoglobulin heavy chain gene. As a result of the translocation, BCL10 protein is over-expressed and localized to the nucleus whereas normal BCL10 expression is found in the cytoplasm of lymphoid cells.

t(14;18)(p32;q21) has not been well studied as mentioned by Remstein et al (9). With this translocation, the MALT1 gene is juxtaposed to the IGH locus. Cases proven to have this translocation have shown not only strong cytoplasmic expression of MALT1 but also of BCL10 (10).

VIII. Chromosome 19q Deletion Probe and Chromosome 1p Deletion Probe

In certain embodiments, the present invention provides a chromosome 19q deletion subtracted probe library, a chromosome 1p deletion subtracted probe library, and a probe pair composed of chromosome 19q deletion and 1p deletion subtracted probe libraries. Preferably, the probe libraries comprise subtracted nucleic acid fragments (e.g. less than 90 or 95% or repeat sequences are present), and are detectably labeled. The chromosome 19q probe library of the present invention may be employed to detect deletions of chromosome 19q, particularly 19q13 (e.g., 19q 13.3 to 19q13.4) in order to diagnose a patient suspected of having a disease such as various glial neoplasms. Example 18 provides one example how chromosome 19q subtracted probe libraries may be generated. The chromosome 1p subtracted probe library of the present invention may be used to select treatment for a patient with a disease, such as cancer. The chromosome 19q and 1p subtracted probe libraries of the present invention may be used in, for example, in situ hybridization methods (e.g. CISH and FISH) in order to screen patient samples for chromosome 19q and/or 1p deletions. In certain embodiments, the chromosome 19q subtracted probe library is approximately 150-200 kb in length (e.g. about 150 kb, about 180 kb, or about 190 kb). In other embodiments, the chromosome 1p subtracted probe library is approximately 200-600 kb in length.

Chromosome 19q deletion occurs in 75% of oligodendrogliomas, 45% of mixed oligoastrocytomas, and 40% astrocytomas (Smith et al., Oncogene, 1999, 18:4144-52, herein incorporated by reference). In these glial neoplasms, a 150 kb minimal region of common deletion (D19S241E-D19S596) spanning 19q13.3-13.4 region has been found (Smith et al., Genomics, 2000, 64:44-50). Loss of an entire copy of 19q is more commonly found in oligodendroglial tumors whereas partial deletion of 19q is more common in astrocytic gliomas. In oligodendrogliomas, deletion of 19q is associated with prolonged overall survival, whereas astrocytomas with 19q deletion is not a statistically significant predictor of overall survival. The chromosome 19q deletion probe of the present invention may be used to aid in the diagnosis of these conditions.

Combined 1p/19q loss has been associated with chemosensitivity and with prolonged survival in patients with oligodendrogliomas (Gelpi et al, Mod. Pathol. 2003, 16:708-15). As such, in certain embodiments, the chromosome 19q probe of the present invention and the chromosome 1p probe of the present invention are used together as a probe pair. These molecular alterations are commonly associated in older children and adults but not in younger children. Pure oligodendrogliomas are more chemosensitive than mixed oligodendrogliomas. This favorable association is not found in patients with astrocytoma or mixed oligoastrocytomas.

As previously reported in oligodendrogliomas, loss of 1p without concomitant loss of 19q is uncommon (Kraus et al., J. Neuropathol. Exp. Neurol. 1995, 54:91-5). Similarly, a tight association was found in oligodendroglial neoplasms of 1p and 19q losses since 96.9% of the cases with 1p alteration also had a 19q alteration whereas only 5.7% had either a 1p or 19q alteration (see Gelpi et al., supra). More importantly, in 1p/19q cases studied, the loss of 1p and/or 19q is a result of the deletion of the whole chromosome arms (Cowell et al., J. Neuropathol., Exp. Neurol., 2004, 63:151-8).

Therefore, since histological classification is limited by the subjective differences in cellular morphology of gliomas with hybrid oligodendroglial/astrocytic features and there are no specific immunohistochemical markers available, there is a need for 1p/19q genetic analysis—which is provided by the chromosome 19q and 1p probes herein. These probes are important for determining appropriate treatment since both histological grading and molecular analysis combined yield more prognostically useful information than either one alone (Fuller et al., J. Neuropathol. Exp. Neurol., 2003, 62:1118-28), and because combined 1p/19q loss is associated with chemosensitivity and with prolonged survival in patients with oligodendrogliomas.

IX. Chromosome 18q Deletion Probe

In certain embodiments, the present invention provides a chromosome 18q deletion subtracted probe library. Preferably, the probe library comprises subtracted nucleic acid fragments (e.g. less than 90 or 95% or repeat sequences are present), and is detectably labeled. The chromosome 18q probe library of the present invention may be employed to detect deletions of chromosome 18q, particularly 18q21 as well as the entire long arm of chromosome 18 in order to diagnose a patient suspected of having a disease such as various cancers (e.g. colorectal, ovarian, cervical, lung, head and neck, and pancreatic cancers). Example 19 provides one example how chromosome 18q subtracted probe libraries may be generated. The chromosome 18q subtracted probe libraries of the present invention may be used in, for example, in situ hybridization methods (e.g. CISH and FISH) in order to screen patient samples for chromosome 18q deletions. In certain embodiments, the chromosome 18q subtracted probe library is approximately 500-575 kb in length (e.g. about 500 kb, about 530 kb, or about 550 kb).

The chromosome 18q probe library targets a particular region on chromosome band 18q21. This probe can be used to identify patients carrying a loss (deletion) of chromosome 18q (long arm), which has been associated with poor prognosis in many human cancers. As a prognostic marker, patients can be grouped and given appropriate therapy.

Chromosomal deletions are common events observed in human cancer. Loss of chromosome arm 18q is among the most frequent cytogenetic alterations seen in colorectal, ovarian, cervical, lung, head and neck, esophageal, and pancreatic cancers (Takebayashi et al., Genes Chromosomes Cancer, 2004, 41:145-54, herein incorporated by reference). Deletion of chromosome 18q21 or the entire long arm has been associated with poor prognosis, tumor progression, and may be linked to the loss of tumor suppressor genes such as DCC (deleted in colorectal cancer) and SMAD4/DPC4 (deleted in pancreatic carcinoma).

Chromosome 18q serves as a prognostic marker as shown by several studies that have linked the loss of Chr.18q with poor survival. It has been demonstrated that chromosome 18q sequences were frequently lost in 73% of colorectal carcinomas (Vogelstein et al., N. Engl. J. Med., 1988, 319:525-32) and predicted poor outcome in patients with stage II colorectal cancer. It has also been shown that approximately 60% of head and neck carcinoma cases have a loss of chromosome 18q and is also linked to poor survival (Takebayashi et al., Cancer Res., 2000, 60:3397-403). As a prognostic marker, this will improve the selection of patients for adjuvant chemotherapy or radiation therapy and select patients who will benefit from the therapy.

X. NF2 (Chromosome 22q) Deletion Probe

In certain embodiments, the present invention provides an NF2 (chromosome 22q) deletion subtracted probe library. Preferably, the probe library comprises subtracted nucleic acid fragments (e.g. less than 90 or 95% or repeat sequences are present), and is detectably labeled. The NF2 subtracted probe library of the present invention may be employed to detect deletions of the NF2 gene or chromosome band 22q (e.g. long arm) in order to diagnose a patient suspected of having a disease such as various tumors (e.g. meningiomas and ependymomas), or identifying patients who are predisposed to develop spinal schwannomas and meningiomas. Example 21 provides one example how NF2 (chromosome 22q) subtracted probe libraries may be generated. The NF2 subtracted probe libraries of the present invention may be used in, for example, in situ hybridization methods (e.g. CISH and FISH) in order to screen patient samples for NF2 gene or chromosome 22q deletions. In certain embodiments, the NF2 subtracted probe library is approximately 300 kb in length (e.g. about 100-500 kb, or about 200-400 kb).

Neurofibromatosis Type 2 (NF2) and NF2 tumor suppressor gene NF2 is an autosomal dominant disorder characterized by the development of bilateral vestibular schwannomas of the eighth cranial nerve and by other brain tumors, including meningiomas and ependymomas (Martuza & Eldridge, N. Engl. J. Med., 1988, 318:684-688). Affected individuals are also predisposed to develop spinal schwannomas and meningiomas (Parry et al., Am. J. Med. Genet., 1994, 52(4):450-61). Genetic linkage and deletion mapping analyses of sporadic and familial NF2-associated tumors showed that inactivation of the tumor suppressor gene in chromosome 22q12, NF2 gene, was involved in the etiology of this disorder (Rouleau et al., Nature, 1987, 329:246-248). The NF2 gene is a tumor suppressor gene (TSG) which encodes a 595-amino acid protein called merlin or schwannomin, a membrane-organizing protein. Previous schwannoma studies have reported diverse frequencies (30-80%) of 22q deletions, involving the NF2 tumor suppressor gene (Bruder et al., Hum. Mol. Genet., 2001, 10:271-282).

Meningiomas are benign tumors of the central nervous system. They are usually sporadic but can also occur associated with the NF2. Monosomy 22 or deletion of 22q have been described in sporadic meningiomas at a high frequency, around 50-70%. Inactivation of the NF2 gene is involved in the pathogenesis of a proportion of meningiomas. Ependymomas arise from the ependymal cells at different locations throughout the brain and spinal cord. NF2 patients are predisposed to the development of ependymomas (Martuza et al.). In adults, allelic loss of chromosome 22q occurs 23-60% of the cases studied, and occur preferentially in intramedullary spinal ependymomas. Some of these tumors also show an altered NF2 gene.

Allelic loss on chromosome 22q are frequently observed in the tumors other than nervous system neoplasms characteristic of the NF2 disorder, such as mesothelioma (Lee and Testa, 1999, Int. J. Oncol., 14:181-188), carcinomas of the colon (Rustgi et al., 1995, Cancer Genet. Cytogenet, 84:24-26), ovary, and breast. Inactivating mutations coupled with allelic loss occur at the NF2 locus is frequent in mesotheliomas. Monosomy 22 or deletion of 22q has also been described in follicular thyroid adenomas and is especially common (46%) in widely invasive follicular carcinoma. LOH of 22q was identified in 29% of pancreatic endocrine tumors. High frequency of loss of heterozygosity (LOH) (77%) on 22q and mutation of NF2 gene were observed independently of c-kit mutation in gastrointestinal stromal tumor (GIST). Since c-kit mutation occurs only in one-third of GIST, there may be other molecular mechanisms involved, e.g. NF2 and maybe other TSGs on 22q.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); DS (dextran sulfate); and C (degrees Centigrade).

### Example 1

### TopoIIα and HER-2/neu Gene Copy Numbers in Breast Cancer Cell Lines

This examples describes the characterization of topoIIα and HER-2/neu gene copy numbers in nine breast cancer cell lines by dual color fluorescent in situ hybridization (FISH) assays. The nine breast cancer cell lines assayed were: BT-474, DU4475, MCF-7, MDA-157, MDA-361, SK-BR-3, ZR-75-1, UACC-812, and UACC-893. A normal human lymphocyte cell line was also used. All cell lines were obtained from the American Type Culture Collection (ATCC, Rockville, Md.). The breast cancer cell lines were grown using recommended culture conditions and harvested at confluency to obtain interphase nuclei from cells that were predominantly in the G1-phase of the cell cycle. The cells were subsequently fixed in methanol:acetic acid (3:1) and placed on microscope slides (see, Tanner et al., Cancer Res., 54:4257 [1994]).

Dual-color FISH experiments were done as known in the art (See e.g., Tanner et al., supra), employing probes for HER-2/neu, topoIIα, and chromosome 17. The HER-2/neu probe employed was P1 clone (RMC17P077) obtained from the Resource for Molecular Genetics (Berkeley, Calif.). A P1 probe for topoIIα was obtained by screening a P1-library (Genome Systems Inc., St. Louis, Mo.). The specificity of the HER-2/neu and topoIIα probes was confirmed by PCR with primers amplifying fragments of HER-2/neu, topoIIα, retinoic acid receptor alpha, and thyroid receptor alpha 1. The following primer sequences were used for topoIIα, 5′-GCCTCCCTAACCTGATTGGTTT-3′ (SEQ ID NO:1), and 5′-CTGAAGAACCCTGAAAGCGACT-3′ (SEQ ID NO:2), resulting in the generation of a 259 base pair PCR product. For HER-2/neu, the following primers were used, 5′-CTGGCTCCGATGTATTTGATG-3′ (SEQ ID NO:3), and 5′-CCTGCCCATAAGTCTCTCTGCT-3′ (SEQ ID NO:4), resulting in the generation of a 210 base pair PCR product. For retinoic acid receptor alpha, the following primers were used, 5′-GATTAGCCTGCCCTCTTTGG-3′ (SEQ ID NO:5) and 5′-CAGAAGGGAGGCAGACAGTC-3′ (SEQ ID NO:6), resulting in the generation of a 148 base pair PCR product. For thyroid hormone receptor alpha 1, the following primers were used, 5′-GCTCATGGTGTCAGGAGGATG-3′ (SEQ ID NO:7), and 5′-GCAGGAATAGGTGGGATGGAG-3′ (SEQ ID NO:8), resulting in the generation of a 196 base pair PCR product. The PCR conditions were optimized for each primer pair for corresponding gene using PTC-100 thermocycler (MJ Research Inc, Watertown, Mass., USA). Approximately 100 ng of each template probe and 25 pmol of corresponding primers were used in a 25 ul reaction volume in a standard reaction mixture recommended for use with DYNAZYME II thermostable DNA polymerase (Finnzymes Oy, Espoo, Finland).

A chromosome 17 pericentromeric probe (p17H8) was used to determine the copy number of chromosome 17. A gene/locus specific probe (HER-2/neu or topoIIα) was hybridized together with the 17 centromere probe. The probes were labeled with biotin-14-dATP and digoxigenin-11-dUTP. The HER-2/neu and topoIIα probes were also hybridized together (one labeled with biotin, another with digoxigenin). After hybridization, the bound probes were detected with avidin-FITC (for the biotin-labeled probe) and anti-digoxigenin rhodamine. Slides were counterstained with 0.2 mm 4,6-diamidino-2-phenylindole (DAPI) in an antifade solution (Vectashield, Vector Laboratories, Burlingame, Calif.).

Hybridization signals were evaluated using an Olympus BX50 epifluorescence microscope equipped with a 63× oil-immersion objective (numeric aperture 1.4). A dual band-pass fluorescence filter (Chromotechnology; Brattleboro, Vt.) was used to separately and simultaneously visualize the FITC and rhodamine signals. Approximately 50 non-overlapping nuclei with intact morphology based on DAPI counterstaining were scored to determine the number of hybridization signals for each of the three probes (i.e., topoIIα, HER-2/neu, and 17 centromere probes). Control hybridizations to normal lymphocyte interphase nuclei were done to ascertain that the probes recognized a single-copy target and that the hybridization efficiencies of the probes used were similar. In these experiments, amplification of HER-2/neu and topoIIα were indicated, if the average ratio of HER-2/neu or topoIIα signals, relative to chromosome 17 centromere signals was 1.5 or more. TopoIIα was considered deleted, in this example, if the ratio was <0.7. The results of this dual color FISH assay are presented in Table 4.

TABLE 4
Absolute and Relative Numbers of topoIIα and HER-2/neu in Breast
Cell Lines

| | | HER-2/neu | | TopoIIα |
|---|---|---|---|---|
| | HER-2/neu | copy number | TopoIIα | copy number |
| | copy number | Relative | copy number | Relative |
| | Absolute | to 17 | Absolute | to 17 |
| Cell Line | (mean ± SD) | centromere | (mean ± SD) | centromere |
| Lymphocytes | 2.0 ± 0.4 | 1.0 | 2.1 ± 0.4 | 1.1 |
| BT-474 | 53 ± 6.2 | 8.0* | 4.2 ± 0.6 | 1.0 |
| DU-4475 | 4.3 ± 0.9 | 1.1 | 4.0 ± 0.4 | 1.0 |
| MCF-7 | 2.7 ± 0.8 | 0.7 | 3.9 ± 0.9 | 1.0 |
| MDA-157 | 3.4 ± 1.1 | 0.9 | 4.0 ± 0.8 | 1.0 |
| MDA-361 | 14 ± 2.3 | 3.5* | 1.9 ± 0.7 | 0.5** |
| SK-BR-3 | 44 ± 6.1 | 7.1* | 9.2 ± 4.8 | 1.5* |
| UACC-812 | 41 ± 7.5 | 10* | 27 ± 5.6 | 6.7* |
| UACC-893 | 66 ± 12 | 32* | 2.3 ± 0.7 | 1.1 |
| ZR-75-1 | 3.3 ± 1.0 | 1.2 | 3.6 ± 0.8 | 1.3 |

*gene amplification of 1.5 or greater;
**physical deletion of less than 0.7.

Of the nine breast cancer cell lines studied, five showed high-level amplification of the HER-2/neu oncogene by FISH. Two of these (UACC-812 and SK-BR-3) showed simultaneous amplification of topoIIα. TopoIIα amplification was found at a low-level of amplification in SK-BR-3, while a high-level of topoIIα amplification was found in UACC-812 cells. The MDA-361 cell line had HER-2/neu amplification with a physical deletion of topoIIα. In the two cell lines with simultaneous amplification of both HER-2/neu and topoIIα (i.e., SK-BR-1 and UACC-812), the copy number of the two genes was not the same. This was unexpected, given the close proximity of these two genes on chromosome 17 and the simple molecular mechanism of amplification of the chromosomal segment carrying these two genes previously suggested (See, Murpy et al., Int. J. Cancer, 64:18 [1996]; and Hoare et al., Br. J. Cancer, 75:275 [1997]), that would yield an identical copy number for the two genes.

### Example 2

### TopoIIα and HER-2/neu Gene Copy Numbers in Primary Breast Cancer Samples

This example describes the characterization of the copy number for HER-2/neu and topoIIα in primary breast cancer samples. One hundred and thirty-six (136) freshly frozen primary breast tumors were derived from the tumor bank at the University of Lund (Lund, Sweeden). HER-2/neu status was previously determined by Southern blotting in 74 of the primary tumor samples (50 samples with reported amplification and 24 samples with reported normal levels of HER-2/neu). Dual color FISH assays were performed on these samples as described above (See, Example 1), in order to detect the HER-2/neu status of each sample.

FISH detection revealed that 47 of the 50 tumor samples with HER-2/neu amplification as determined by Southern blot also showed amplification by FISH. Also, four low-level HER-2/neu amplifications were identified by FISH in the 24 samples reported to have normal levels of HER-2/neu by Southern Blotting. In the 62 remaining samples, 19 amplifications and one physical deletion were detected by FISH. The total number of HER-2/neu amplifications found, therefore, was 70 out of 136, with an average gene copy number per cell of 21.7±12.2.

The gene copy numbers of topoIIα was then determined by FISH (See, e.g., Example 1) on the 70 primary breast cancer samples determined to have HER-2/neu amplification. Twenty-nine of these tumors (41%) were found to have simultaneous amplification of topoIIα and HER-2/neu (with a mean of 12.7±6.4 and 19.6±10.3 gene copies/cell respectively). In these 29 tumors with amplification of both HER-2/neu and topoIIα, the mean number of HER-2/neu copies was higher than that of topoIIα in 15 tumors (52%), the copy numbers were equal in only 10 tumors (34%), and the topoIIα copy number exceeded the HER-2/neu copy number in 4 tumors (14%). The fact that the copy number of the two genes was not the same in all of the tumor samples (only the same in 34%) was unexpected. This result is unexpected given the close proximity of these two genes on chromosome 17 and the simple molecular mechanism of amplification of the chromosomal segment carrying these two genes.

### Example 3

### Characterization of TopoIIα-HER-2/neu Amplification by Fiber FISH

This example describes the characterization of topoIIα-HER-2/neu amplification by fiber FISH in the UACC-812 cell line. Mechanically extended DNA fibers were prepared from UACC-812 cells by first embedding the cells in 0.9% agarose (See, Heiskanen et al., Genomics, 30:31 [1995]). A small piece of the agarose block was placed on a poly-L-lysine-coated (Sigma) microscope slide and heated on a 95° C. hot plate for 20 seconds. The melted agarose was spread along the microscope slide mechanically with another microscope slide and air dried for 30 minutes. This resulted in the extension of the DNA fibers. The fiber-FISH (for topoIIα and HER-2/neu) was carried out according to the same procedure as described in Example 1 above for FISH. However, proteinase K digestion of the target DNA was omitted and hybridization efficiency was increased by applying denatured probes on the denatured target DNA and re-denaturing them together on a hot plate at approximately 95° C. for 1.5 minutes.

The results of this fiber-FISH analysis revealed that amplified HER-2/neu and topoIIα gene copies were localized exclusively in overlapping clusters in five marker on chromosomes, although chromosomal regions with HER-2/neu signals were also seen. Fiber-FISH was used to characterize the amplicon at high resolution. Surprisingly, HER-2/neu and topoIIα signals were found in separate DNA fibers. Signals for both genes were repeated with themselves, but not with each other, indicating two different tandem repeat-like amplification units. The successive signals for both HER-2/neu and topoIIα were at a constant length from each other, suggesting that the same region was repeatedly amplified. For confirmation of the separate amplicons for HER-2/neu and topoIIα genes, individual nuclei from which separate DNA fibers with either repeated HER-2/neu or topoIIα signals originated were found.

### Example 4

### TopoIIα Gene Status does not Correlate with Immunohistochemistry

This example describes the lack of correlation between topoIIα gene status and immunohistochemical (IHC) detection of protein. In particular, 34 primary breast cancer samples were assayed for topoIIα gene status (employing FISH), and for the presence of topoIIα protein (employing antibody detection). The FISH detection was carried out as described in Example 1. Immunohistochemical analysis started with 5 um sections of the primary breast cancer samples that were cut and mounted on SuperFrost slides and dried overnight at 37° C. The sections were then dewaxed and rehydrated. Antigen retrieval of paraffin embedded, formalin fixed tissue sections was done by heating in a microwave for 2-7 minutes in citrate buffer (pH 6.0). TopoIIα monoclonal antibodies Ki-S4 (Kellner et al., J. Histochem. Cytochem, 45:251 [1997]) were incubated with the breast cancer sample for 25 minutes at room temperature. The bound antibodies were visualized using a streptavidin-biotin-peroxidase kit (Vector Labs, Burlingame, Calif.) with diaminobenzidine as the chromogen. Methyl green was used for counterstaining. Immunoreaction was quantitated with a CAS200 image analysis system. The obtained scores were tabulated as a percentage of immunopositive nuclei.

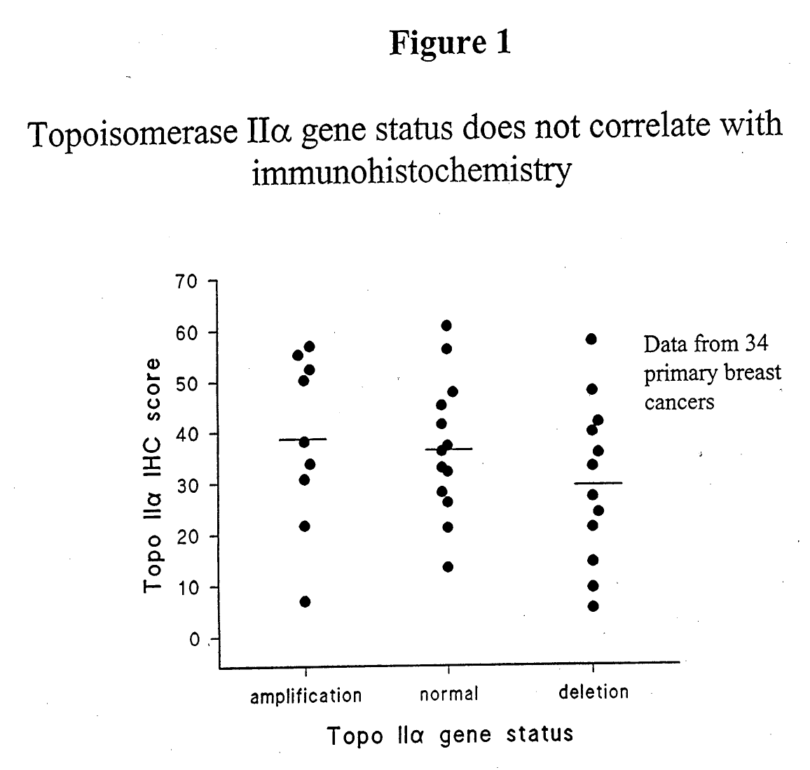

The result of the IHC and FISH detection in these breast cancer samples is presented in FIG. 1. The dramatic and unexpected results presented in this Figure indicate that the presence of TopoIIα in the samples as determined by IHC does not correlate with the gene copy status of topoIIα as determined by FISH. FIG. 1 indicates that the presence of topoIIα in the breast cancer samples was essentially independent of topoIIα gene status. In other words, these results demonstrate that topoIIα gene copy number cannot be effectively determined by relying on IHC techniques.

### Example 5

### HER-2/neu Amplification is not Significantly Associated with Clinical Response to Chemotherapy

This example describes the characterization of HER-2/neu copy number in 191 primary breast cancer tissue samples and the lack of association of HER-2/neu copy number with clinical response to chemotherapy. In particular, the 191 breast cancer tissue samples were obtained from patients who took part in a previously reported prospective randomized trial, where single agent epirubicin chemotherapy was compared with an epirubicin-based combination regimen (CEF—cyclophosphamide, epirubicin, and 5-fluorouracil) as first-line chemotherapy for advanced breast cancer (Joensuu et al., J. Clin. Oncol., 16:3720 [1998]). Briefly, patients eligible for this previous study were required to have distantly metastasized breast carcinoma, with the presence of distant metastases confirmed histologically, cytologically, or radiologically. Patients who had received prior cytotoxic chemotherapy for metastatic disease or anthracyclines in the adjuvant setting were not eligible for the study. Patients with brain or leptomeningeal metastases, those with the World Health Organization (WHO) performance status greater than 2, and those older than 70 years at randomization were also excluded. Clinical examination, imaging and laboratory examinations were carried out before randomization and during follow-up.

In this previous study, patients assigned to combination chemotherapy received CEF (cyclophosphamide 500 mg/m², epirubicin 60 mg/m², and 5-fluorouracil 500 mg/m²) intravenously at 3-week intervals as first-line chemotherapy, and MV (mitomycin C 8 mg/m², combined with vinblastine 6 mg/m²) at 4-week intervals as second-line chemotherapy. Patients assigned to the single agent arm were treated weekly with single-agent epirubicin at 20 mg/m² as first-line therapy. After disease progression or reaching a maximum cumulative dose of epirubicin, single-agent mitomycin C 8 mg/m² was given 4 times weekly as second-line therapy. Local radiotherapy for painful metastatic lesions, bisphosphonate therapy, and anti-nausea medication were allowed at any time during the study. Responses to first-line chemotherapy were evaluated during regular follow-up visits to the oncology clinic. The clinical response was classified into 4 categories; “complete response” (CR), “partial response” (PR), “no change in disease progression” (NC), and “progressive disease” (PD) according to the WHO criteria (Miller et al., Cancer, 47:207-214 [1981]).

The response rates, reported in this previous study, to CEF (CR+PR, 55%) and to single-agent epirubicin (CR+PR, 48%) were statistically not different (p=0.21) in this trial, and overall survival was also similar. Because epirubicin was the only topoisomerase II inhibitor agent in both first-line treatments and because its cumulative dose was similar in both arms (471 mg/m² in the CEF arm and 444 mg/m² in the single-agent epirubicin arm), the two treatment groups were combined and analyzed as a single group for predictive correlations in the present example. Of the 303 patients randomized in the trial, archival paraffin-embedded and histopathologically representative samples (containing >50% carcinoma cells) from the primary tumor were available from 196 patients. HER-2/neu FISH was carried out on 191 of these samples as described below.

FISH was performed using a digoxigenin-labeled probe for HER-2/neu obtained from Zymed Inc. (South San Francisco, Calif.). Pretreatment of paraffin sections was carried out using a SPOT-LIGHT FFPE reagent kit from Zymed Inc. Briefly, sections were de-paraffinized and incubated in Pretreatment Buffer in a temperature-controlled microwave oven (at 92° C. for 10 min). Enzymatic digestion was carried out with FFPE digestion enzyme (10 to 40 min at room temperature). The slides were washed with PBS and dehydrated in graded dilutions of ethanol. The HER-2/neu probe was then applied to the slides. The slides were denatured on a hot plate (94° C.) for 3 min and hybridized overnight at 37° C. After hybridization, the slides were stringency washed with 0.5×SSC (5 min at 75° C.), followed by three washes in PBS/0.2% Tween20. The HER-2/neu probe was detected with anti-digoxigenin rhodamine (diluted 1:300, Roche-Boehringer, Mannheim, Germany). Nuclei were counterstained with 0.1 uM 4,6-diamidino-2-phenylindole (DAPI) in an antifade solution (Vectashield, Vector Laboratories, Burlingame, Calif.).

Hybridizations were evaluated using an Olympus BX50 epifluorescence microscope. Signals from at least 50 to 200 non-overlapping nuclei with intact morphology were evaluated to determine the mean number of signals/cell for each probe. Absolute copy numbers for HER-2/neu were then determined. Amplification of HER-2/neu was defined, in this example, as the presence of 6 or more copies of HER-2/neu in over 50% of nuclei. All analyses were carried out in a blinded fashion (i.e. without knowing the clinical response or survival). HER-2/neu gene amplification, as defined in this example, was observed in 61 of the 191 tumors tested (i.e., 31.9%).

Amplification of HER-2/neu was found to be associated with a negative hormone receptor status and p53 overexpression, but there was no significant association between the presence of HER-2/neu amplification and the primary tumor size, axillary lymph node status or the dominant site of metastasis. HER-2/neu amplification was significantly associated with a short distant disease-free interval, and overall cancer-specific survival.

In regards to HER-2/neu status and previously reported response to epirubicin-based chemotherapy, no significant correlation was found. A comparison of HER-2/neu status and response to epirubicin-based chemotherapy is presented in Table 5.

TABLE 5
Association of HER-2/neu Gene Status and Response to Chemotherapy

| | | | | Progres- | Not |
|---|---|---|---|---|---|
| Response to | Complete | Partial | | sive | evalu- |
| Chemotherapy | response | response | No change | disease | able |
| No HER-2/neu | 6 (4.6%) | 61 (47%) | 36 (28%) | 23 (18%) | 4 (3%) |
| amplification | | | | | |
| HER-2/neu | 7 (11%) | 18 (30%) | 11 (18%) | 18 (30%) | 7 (11%) |
| amplification | | | | | |

These results demonstrate that there is no significant correlation between the HER-2/neu amplification status and the clinical response to first-line chemotherapy. This is evidenced by the fact that the prevalence of HER-2/neu amplification was not significantly different between responders (CR or PR) and non-responders (NC or PD) (p=0.42).

### Example 6

### Predictive Value of TopoIIα and HER-2/neu Amplification in Primary Tumors Cells-FISH TopoIIα Detection

This example describes the predictive value of dual amplification of topoIIα and HER-2/neu in regards to clinical response to topoisomerase II inhibitor chemotherapy. In particular, FISH was used to determine the topoIIα gene copy number for the 61 tumor samples (i.e., primary cells) determined to have HER-2/neu amplification in Example 5 (See, Table 5).

PAC clones probe for topoIIα were obtained by PCR-based screening of a PAC library. A chromosome 17 pericentromeric probe (p17H8) was used as a reference probe to determine the overall copy number of chromosome 17. The specificity of the topoIIα probe was confirmed by PCR with topoIIα specific primers. This topoIIα probe does not contain HER-2 DNA sequence since there is no amplification using 3 pairs of HER-2 specific primers covering 5′ end, middle, and 3′ end of HER-2. The PCR-analysis showed that the topoIIα probe did not recognize sequences from HER-2/neu. The pericentromeric probe for chromosome 17 was labeled with fluorescein-5-dUTP and the topoIIα probe with digoxigenin-11-dUTP by standard nick-translation. A mixture of the topoIIα and 17 centromere probes (30 ng and 10 ng, respectively) was diluted in 10 ul of hybridization buffer (2× standard saline citrate (SSC), 50% formamide, 10% dextran sulfate), and applied to the slides under coverslips.

In this Example, control hybridizations to non-malignant breast tissue and normal peripheral blood lymphocytes were also carried out to ascertain the relative hybridization efficiencies of topoIIα and 17 centromere. The sensitivity of FISH in the detection of aberrations of topoIIα when using paraffin sections was validated with a separate set of 15 tumors in which freshly frozen tumor material had been analyzed previously by FISH. TopoIIα amplification was defined, in this example, as a copy number ratio of 1.5 or more, and deletion was defined, in this example, as a ratio of 0.7 or less

TopoIIα amplification (as defined in this Example) was found in 21 (34%) tumors, 27 (44%) had no topoIIα copy number alterations, and 13 (21%) showed topoIIα deletion (as defined in this example). The median number of topoIIα gene copies per cell in tumors with amplification was 14 (the median number for HER-2/neu gene copies was 25/cell). In tumors with topoIIα deletion, the median number of gene copies was 2.3 (4.3 for chromosome 17 centromere; the average copy number ratio was 0.53).

In regards to topoIIα gene status in HER-2/neu positive breast cancer samples and previously reported response to epirubicin-based chemotherapy, a significant correlation was found. A comparison of topoIIα to gene status in the HER-2/neu positive samples (gene amplification) and response to epirubicin-based chemotherapy is presented in Table 6.

TABLE 6
Association of TopoIIα Gene Aberrations with Clinical Response To
Chemotherapy in 61 HER-2/neu Positive Breast Cancer Samples

| | | | | Progres- | |
|---|---|---|---|---|---|
| Response to | Complete | Partial | | sive | Not |
| Chemotherapy | response | response | No change | disease | evaluable |
| TopoIIα | 7 | 8 | 2 | 2 | 2 |
| amplification | | | | | |
| Unaltered | 0 | 8 | 5 | 10 | 4 |
| TopoIIα | 0 | 2 | 4 | 6 | 1 |
| Deletion | | | | | |

These results indicate that topoIIα aberrations were strongly associated with clinical response to first-line epirubicin-based chemotherapy. Significantly, all seven patients who had a complete response to anthracycline chemotherapy had a primary tumor with topoIIα and HER-2/neu amplification. Fifteen (79%) of the 19 evaluable patients with topoIIα and HER-2/neu amplification achieved either a complete or partial response to chemotherapy (i.e., were identified as suitable for treatment with topoisomerase II inhibitors). In contrast, only 8 of the 23 (35%) evaluable patients with an unaltered topoIIα status and 2 of the 12 (17%) patients who had cancer with topoIIα deletion responded to epirubicin-containing chemotherapy. Also, the duration of response was significantly longer in patients with topoIIα amplification than in those with deletion or with unaltered topoIIα (median 10 vs. 5 months, p=0.01).

TopoIIα alterations were not associated with the length of long term disease-free survival following breast surgery, (i.e., not influenced by chemotherapy that was given for metastatic disease). In agreement with the association to favorable clinical response, topoIIα amplification together with HER-2/neu amplification was significantly associated with improved post-chemotherapy survival as compared to patients who had cancer with an unaltered topoIIα gene copy number or topoIIα deletion (median 20 vs. 11 months).

### Example 7

### Generating HER2/neu Subtracted Probe Library

This example describes the generation of an exemplary HER2/neu subtracted probe library. This exemplary probe is useful in in-situ hybridization techniques such as CISH and FISH.

1. Selection of the BAC Clone for Detection of HER2 Gene Amplification.

Two BAC clones (312L7 and 359J8) were identified by PCR screening of a human BAC library (obtained from Research Genetics). These two BAC clones contain both the 5′ and 3′ ends of HER-2 gene. The 5′ and 3′ ends of the two BAC clones were sequenced, and a BLAT search using these sequences was performed. In the UCSC Genome Browser Dec. 22, 2001 Freeze, it was determined that BAC clone 3127L is located at 39829991-39946716 (117 kb), and the BAC clone 359JB is located at 39890898-40010140 (119 kb). The HER2 gene is located at 39915101-39943629. As such, both BAC clones contain the HER2 gene. FISH using each clone showed both of them bind specifically to the HER-2 gene locus on chromosome band 17q21 and absence of chimerism. The FISH signal generated using the two clones together was larger than that generated using either clone on its own.

2. Preparation of Tracer DNA.

The tracer DNA, which is used to generate a library of HER2 probe for ISH, was prepared by sonication of 10 ug of the purified BAC clone DNA to 0.1-8 kb and size-fractionated on 1% agarose gel. The 0.5-4 kb fractions were cut from the gel, purified using QIAquick gel extraction kit (QIAGEN, Santa Clarita, Calif.), blunt ended and ligated to the adapter. The T1/T2 adapter was constructed by annealing polyacrylamide gel electrophoresis (PAGE)-purified oligos T1 5′-CTG AGC GGA ATT CGT GAG ACC-3′ (SEQ ID NO: 18) (“sense” oligo), and T2, 5′-PO4 GGT CTC ACG AAT TCC GCT CAG TT-3′ (SEQ ID NO:19) (“antisense” oligo).

Adapter ligated fragments (100 ng) were then PCR amplified, in multiple 25 ml reactions, using the T1 sequence as primer. PCR cycling conditions were 94° C. for 30 sec, 60° C. for 30 second and 72° C. for 3 min for 30 cycles, followed by 72° C. for 10 minutes. Amplified fragments were size-fractioned (0.5-4 kb fractions) on 1% agarose gel, then purified using QIAquick gel extraction kit.

3. Preparation of Biotin-Labeled Driver DNA

Human high molecular weight Cot-1 (3 mg) was blunt ended and ligated to the D40/D41 adapter constructed by annealing PAGE-purified oligos (SEQ ID NOS: 15 and 16 respectively). Adapter ligated fragments (100 ng) were then PCR amplified, in multiple 25 ul reactions, using 5′end biotin-labeled D40 (D40B, SEQ ID NO:17) sequence as primer. PCR cycling conditions were 94° C. for 30 sec, 60° C. for 30 sec and 72° C. for 3 min for 30 cycles, followed by 72° C. for 10 min. The PCR product was purified by phenol:chloroform:isoamyl. The pellet was dried and drive DNA carefully re-dissolved at 1.5-2.5 mg/ml in EE buffer (10 mmol/L 2hydroxyethyl] piperazine-N′-3-propanesulfonic acid (NaEPPS), 1 mmol/L EDTA, pH 8.0).

4. Subtraction Hybridization

Genomic subtractive hybridization removed sequences from a tracer DNA population by hybridizing with a molar excess of driver DNA. The driver DNA is chemically modified, with a biotin, such that it may be selectively removed from solution along with driver-tracer hybrid molecules. Briefly, HER2 tracer DNA was repeatedly hybridized with 40-fold excess of biotin-labeled Driver DNA containing the repetitive sequences (Alu and LINE elements). Consequently, repetitive sequences presenting the HER2 region were quantitatively removed. The detailed methods are set forth below.

Subtraction was performed by mixing 250 ng of tracer DNA with 10 mg of biotin-labeled driver DNA, 2 mg of T1, 5 mg of yeast tRNA as carrier. This mixture was denatured at 100° C. for 2 min, lyophilized, re-dissolved in 5 ml of EE buffer/1 mol/L NaCl, then incubated at 65° C. for 24 to 48 hours. Biotinylated molecules (including tracer-driver hybrids) were removed using avidin-polystyrene beads. Remaining unbiotiylated tracer fragments were precipitated in ethonal before proceeding with the next round of subtraction. Each of three rounds of subtraction was performed as described above. After the third round, remaining tracer fragments were amplified by PCR using the T1 sequence as primer.

5. Probe Library Preparation

After three rounds of subtraction and 3 rounds of PCR, the HER2 DNA probe library was labeled with digoxigenin (DIG) using random octamer primer kit (Gibco BRL/Life Technologies). The final nucleotide concentrations for DIG labeling were 0.2 mmol/L dCTP, dGTP, dATP, 0.13 mmol/L dTTP, and 0.07 mmol/L Dig-11-dUTP (Boehringer Mannheim/Roche). Residual primers and unincorporated nucleotides were removed by S-200HR spin column chromatography (Amersham Pharmacia Biotech Inc.). The purified products were precipitated in ethanol and dissolved in a solution containing 50% formamide, 10% dextran sulfate, and 2×SSC (0.3 mol/L sodium chloride, 0.03 mol/L sodium citrate, pH 7.0).

### Example 8

### Chromogenic In-Situ Hybridization Detection of HER2/neu

This example describes performing chromogenic in-situ hybridization with the HER2/neu probe library generated in Example 7, as well as general procedures for evaluating CISH results. CISH was done on 4 μm-thick tissue sections mounted on Superfrost/plus microscope slides (Fisher, Pittsburgh, Pa.). The slides were baked 2-4 hours at 65° C. and then deparaffinized 10 minutes in Xylene (2 times) and 5 minutes in ethanol (3 times). Air-dried tissue sections were placed in a plastic Coplin jar containing the CISH Pretreatment Buffer (0.1M Tris/0.05 M EDTA, pH 7.0, SPOT-Light Tissue Pretreatment Kit, Zymed), and loosely capped. They were heated at 199° F. for 15 min in the microwave with a temperature probe (GE Profile Sensor convection). The temperature probe was placed in a separate plastic Coplin jar without a cap. As a result of capping the Coplin jar, the tissue sections reached a boiling temperature (100 degrees Celsius), which was evidenced by the solution in the jar boiling when the microwave was stopped and the jar examined.

The slides were washed immediately with deionized water after heat pretreatment. Enzyme digestion was followed by covering the section with prewarmed 37° C. pepsin (0.0625% pepsin, pH 2.3, SPOT-Light Tissue Pretreatment Kit, Zymed) and by incubating at 37° C. for 3±1 minutes. The slides were then washed with deionized water, dehydrated with graded ethanol, and air-dried. The ready-to-use DIG-labeled HER2 probe (See, Example 7) or biotin-labeled chromosome 17 centromeric probe (SPOT-LIGHT Chromosome 17 Centromeric Probe, Zymed Laboratories, Inc.) was applied to the center of the coverslip. The coverslip was placed with probe side down on the tissue sample. 15 μl or 20 μl of the probe was used for 22×22 mm or 24×32 mm coverslips according to the size of the tissue sections to be covered. After sealing the edges of the coverslips with rubber cement, the tissue sections and the probes were denatured at 94° C. for 5 minutes by placing the slides in the slide block of the PCR machine (MJ research, Watertown, Mass.). Hybridization was done in the same slide block at 37° C. overnight. The stringent wash was done with 0.5× standard saline citrate at 75-80° C. for 5 minutes.

Next, the endogenous peroxidase activities were blocked in 3% H₂O₂ diluted with methanol for 10 minutes. The unspecific staining was blocked by applying the Cas-Block™ (0.25% casein, 0.2% gelatin, and 10 mM PBS, pH 7.4) on the tissue section and by incubating for 10 minutes. After blotting off the Cas-Block™, FITC conjugated mouse anti-DIG antibody was applied on the tissue section and incubated for 45 minutes at room temperature. After three times washing, each 2 minutes with PBS and Tween 20, HRP conjugated sheep anti-FITC antibody was applied on the tissue section and incubated for 45 minutes at room temperature, followed by DAB development for 30 minutes. The biotin-labeled chromosome 17 centromere probe was detected with sequential incubation with HRP conjugated streptavidin for 45 minutes at room temperature and DAB development (CISH Centomere Detection Kit, Zymed) for 30 minutes. Tissue sections were counterstained with hematoxylin, dehydrated, and coverslipped. Positive controls were included in each staining run.

Evaluation of CISH results. CISH results were evaluated using a bright field microscope (Nikon, E400) equipped with 10×, 20×, and 40× dry objectives with 10× oculars (see Table 7). For evaluation of HER2 CISH results please see Table 8. An individual HER2 gene or chromosome 17 centromere signal appears as a small, single dot. Targeted HER2 gene amplification is typically seen as large DAB-stained clusters or many dots in the nucleus.

TABLE 7
Signal Visualization

| Magnification | CISH Signal |
|---|---|
| 10× | Individual signals are barely visible and may be |
| | missed. |
| 20× | Individual signals are small but clearly discernible. |
| 40× | Individual signals are easily identified. |
| 60× or 100× | Not necessary |

TABLE 8
Exemplary Criteria of HER2 gene status by CISH

| | |
|---|---|
| Amplification | >10 copies or large clusters of HER2 gene (amplicon) per |
| | nucleus in >50% of cancer cells. |
| Low | 6-10 copies of HER2 gene or small cluster of HER2 |
| Amplification | gene (amplicon) per nucleus in >50% of |
| | cancer cells. |
| | Biotin-labeled Spot-Light chromosome 17 centromeric |
| | probe may be applied for CISH to confirm that 6-10 |
| | copies of HER2 gene (<5% cases) were due to |
| | HER2 gene amplification but not chromosome |
| | 17 polysomy. |
| No | 1-5 copies of HER2 gene per nucleus in >50% of cancer |
| Amplification | cells. |
| | 3-5 copies of HER2 gene per nucleus is due to |
| | chromosome 17 polysomy. There is no need for |
| | chromosome 17 centromeric CISH. |
| | Occasionally, it is found that HER2 has 3-5 copies and |
| | chromosome 17 centromere has 1-2 copies in >50% of |
| | cancer cells (HER2/chr.17cen ratio is ≧2), it is due to |
| | what sometimes was seen by CGH of duplication |
| | of chromosome arm 17q. |

The CISH staining results are clearly seen using a 40× objective in tissue sections which are counterstained with, for example, hematoxylin. An individual gene or chromosome centromere signal appears as a small, single dot. Targeted gene amplification is typically seen as large DAB-stained clusters or many dots in the nucleus or mixed clusters and multiple dots (≧6 dots per nucleus). Tumors with no targeted gene amplification show typically 1 to 5 dots per nucleus. 3-5 dots per nucleus in more than 50% of tumor cells are due to chromosome polysomy.

### Example 9

### Chromogenic In Situ Hybridization (CISH) Detection of HER-2/neu

This example describes chromogenic in situ hybridization (CISH) detection of HER-2/neu in primary breast cancer samples, as well as a comparison between CISH, FISH, and IHC detection of HER-2/neu gene copy number or HER-2 protein. One-hundred and fifty-seven (157) tumor samples were employed in this example, and were collected prospectively at the Jules Bordet Institute.

CISH was performed on 5 mm thick archival formalin-fixed paraffin-embedded tissue sections. In brief, the sections were de-paraffinized and incubated in pretreatment buffer in a temperature-controlled microwave oven (at 92° C. for 15 minutes, using a SPOT-LIGHT FFPE reagent kit from Zymed Inc., (South San Francisco, Calif.). The sections were then washed three times with deionized water. Enzymatic digestion was done by applying 100 ul of FFPE digestion enzyme on to slides (10-15 min at room temperature). The slides were then washed with PBS and dehydrated with graded ethanols. The ready-to-use digoxigenin-labeled HER-2/neu probe (Zymed, consisting of two contig BAC clones) was applied onto slides which were covered under 14×14 mm coverslips (10 ul probe mixture/slide). The slides were denatured on a hot plate (94° C.) for 3 min, and the hybridization was carried out overnight at 37° C. After hybridization, the slides were washed with 0.5×SSC (standard saline citrate; 5 min at 75° C.), followed by three washes in PBS/0.025% Tween20 (at room temperature). The HER-2/neu probe was detected with sequential incubations with anti-digoxygenin-fluorescein, anti-fluorescein-peroxidase and diaminobenzidine according to manufacturer's instructions (Zymed Inc.). Tissue sections were lightly counterstained with hematoxylin and embedded.

The CISH hybridizations were evaluated using an Olympus BX50 microscope equipped with 40× and 60× dry objectives using 10×22 widefield oculars. Unaltered gene copy number was defined, in this example, as 1 to 5 signals per nucleus. Low level amplification was defined, in this example, as 6 to 10 signals per nucleus in over 50% of cancer cells, or when a small gene copy cluster was found. Amplification of HER-2/neu was defined, in this example, when a large gene copy cluster in over 50% of carcinoma cells, or numerous (>10) separate gene copies were seen. Images were captured using a Pixera PVC100C digital camera (Pixera Corp., Los Gatos, Calif.).

In this example, FISH was done as previously described (Grancberg, et al., Am. J. Clin. Pathol., 113:675 [2000]). In brief, a fresh tumor sample of 0.5 cm³ of a freshly made imprint touch preparation were obtained immediately after surgery. Cells from tumor pieces were mechanically disintegrated, centrifuged and treated with 0.075M KCl for 1 h at 37° C. After washing in methanol:acetic acid (3:1), the cells were spread onto microscope slides. The slides were denatured in 70% formamide/2×SSC (pH 7) at 73° C. for 10 min. After dehydration in an ethanol series, 10 ul of the probe (LSI HER-2/CEP17, Vysis Inc., Downers Grove, Ill.) was denatured (73° C. for 5 min) and applied onto slides. The hybridization was carried out overnight at +37° C. in a moist chamber. The samples were washed in 0.4×SSC (at 73° C., 2 min), followed by 0.4×SSC/0.1% Nonidet P-40 (2 min at room temperature) to remove excess probes. Nuclei were counterstained with 4′,6-diamino-2 phenylindole dihydrochloride (DAPI, 1 mg/ml) in an antifade embedding solution (p-phenylene-diamine dihydrochloride).

Hybridization signals were enumerated in at least 150-250 morphologically intact and non-overlapping nuclei. A Leica DMRB epifluorescence microscope equipped with a 100× oil immersion objective and a triple bandpass filter was employed for simultaneous detection of Spectrum Green, Spectrum Orange and DAPI (filter from ChromaTechnology, Tucson, Ariz.). Her-2/neu amplification was determined as a ratio of HER-2/neu and chromosome 17 centromere signal counts. Ratios below 2 were defined, for this example, as “no amplification,” those between 2 and 5, were defined for this example, as “low level amplification,” and those above 5, were defined for this example, as “high level amplification.”

Immunohistochemistry (IHC) of HER-2 was done on tissue sections adjacent to those used in the CISH detection described above. The sections were de-paraffinized followed by antigen-retrieval in 0.01 M citrate buffer (pH 7.3, 94° C. for 20 min, using a temperature-controlled microwave oven). After blocking for non-specific antibody binding (using the blocking reagent Histostain Plus kit), the sections were incubated overnight (at 4° C.) with a monoclonal antibody to the intracellular domain of HER-2 protein (clone CB-11, Novocastra Laboratories, Newcastle UK). A standard avidin-biotin-peroxidase complex (ABC) technique was used for visualization, with diaminobenzidine as the chromogen (Histostain Plus-kit, Zymed Laboratories, San Francisco, Calif.). Intense cell membrane immunoreaction present in over 50% of cancer cells was designated as “3+” staining and was considered as overexpression of HER-2. Staining present in a smaller proportion of cells or that with lower intensity was designated as “2+” staining. The controls consisted of three cell lines (SK-BR-3; >30 gene copies of HER-2/neu, MDA-MB-453; 8 gene copies of HER-2/neu, and ZR-75-1, 2 gene copies of HER-2/neu) were fixed overnight with 10% formalin and pelleted as a normal paraffin block.

Results obtained by CISH and FISH performed on cells prepared from a fresh tumor sample were correlated. In a series of 157 unselected breast cancers, the prevalence of HER-2/neu amplification was determined to be 23.6% by FISH and 17.2% by CISH. There were 120 tumors with no amplification and 27 with amplification by both methods (Table 9). FISH identified HER-2/neu amplification in 10 tumors which were negative by CISH (5 gene copies or less) (Table 9). The kappa coefficient (measuring agreement between the methods, 0=no agreement, 1=perfect agreement) was 0.81 (95% confidence interval 0.69-0.92).

TABLE 9
Comparison Between CISH and FISH Detection of HER-2/neu
Copy Number

| | CISH - No amplification | CISH - Amplification |
|---|---|---|
| FISH - No | 120 (76.4%) | 0 (0%) |
| amplification | | |
| FISH - Amplification | 10 (6.4%) | 27 (17.2%) |

HER-2/neu gene amplification by CISH and FISH was also compared with HER-2 protein overexpression detected by immunohistochemistry (using monoclonal antibody CB-11) (Table 5). Immunohistochemistry was somewhat less sensitive but generally in good agreement with FISH and CISH. The prevalence of HER-2 overexpression was 19.7% as determined by immunohistochemistry. There were 11 tumors positive by FISH but negative by IHC, but only 2 such tumors positive by CISH. Only one of the immunohistochemically weakly positive (2+) tumors were found to be amplified using CISH or FISH.

TABLE 10
FISH and CISH HER-2/neu Analysis Compared to IHC HER-2 Analysis

| | IHC - Negative | IHC - Weakly | IHC - positive |
|---|---|---|---|
| | (0 or +1) | positive (2+) | (3+) |
| FISH - No | 115 | 4 | 1 |
| amplification | | | |
| FISH - Amplification | 11 | 1 | 25 |
| CISH - No | 124 | 5 | 1 |
| amplification | | | |
| CISH - Amplification | 2 | 0 | 25 |

As described above, the agreement between CISH with FISH was generally very good. However, there were 10 tumors (6.4%) defined, in this example, as amplified by FISH but not amplified (as defined in this example) by CISH (See, Table 9). One explanation for this difference is the sample materials. FISH was done on fresh tissue material, whereas CISH was conducted using paraffin-embedded samples, which are technically more difficult to hybridize. A second explanation, examining the discordant tumors in detail (See Table 11), it appears that all but one tumor (that was negative by CISH) was scored as having a borderline ‘low level’ amplification in FISH (copy number ratio 2 to 5). Moreover, eight of these tumors were negative by immunohistochemistry (one had 2+staining). Thus, the discrepancies may simply reflect the fact that the threshold for determining low level amplification as used in this example may not always clearly detect HER2 overexpression.

TABLE 11
Results of HER-2/neu CISH, FISH, and IHC in Cases with Disagreement

| Tumor | | | |
|---|---|---|---|
| No. | FISH | CISH | IHC |
| #22 | Low level amplification | Not amplified | Negative (0 or 1+) |
| #41 | Low level amplification | Not amplified | Negative (0 or 1+) |
| #52 | Low level amplification | Not amplified | Negative (0 or 1+) |
| #54 | Low level amplification | Not amplified | Negative (0 or 1+) |
| #88 | High level amplification | Not amplified | Negative (0 or 1+) |
| #106 | Low level amplification | Not amplified | Weakly positive |
| | | | (2+) |
| #123 | Low level amplification | Not amplified | Negative (0 or 1+) |
| #126 | Low level amplification | Not amplified | Negative (0 or 1+) |
| #127 | Low level amplification | Not amplified | Negative (0 or 1+) |
| #135 | Low level amplification | Not amplified | Negative (0 or 1+) |

### Example 10

### Exemplary TopoIIα Probe and Other TopoIIα Probes

This Example describes an Exemplary TopoIIα probe useful for detecting TopoIIα copy number in, for example, FFPE tissue sections, fresh tissue sections, cell preparations, and metaphase chromosome spreads using in situ hybridization detection methods such as FISH and CISH. This Example also describes procedures for constructing similar probes.

The Exemplary TopoIIα probe described in this Example is available from Zymed Laboratories (South San Francisco, Calif., Cat. No. 84-0600) as a library of fragments ranging in size from about 0.5 to 4 kb in size. The nucleic acid sequence of the Exemplary TopoIIα probe is an approximately 170 kb sequence from human chromosome seventeen (17) that encompasses the TopoIIα gene, but does not contain the HER2/neu gene. FISH experiments revealed that the probe binds specifically to the topoIIα gene locus on chromosome band 17q11-21 and absence of chimerism. PCR with HER2/neu specific primers demonstrated that the sequence of the Exemplary TopoIIα probe does not contain the HER2/neu gene.

i) Selection of PAC Clone for Detection of TopoIIα Gene Amplification

In order to isolate the PAC clone for topoIIα, PAC clones probes for topoIIα were obtained by PCR-based screening of a PAC library. A chromosome 17 pericentromeric probe (p17H8) was used as a reference probe to determine the overall copy number of chromosome 17. The specificity of the topoIIα probe was confirmed by PCR with topoIIα specific primers. This topoIIα probe does not contain HER-2 DNA sequence since there is no amplification using 3 pairs of HER-2 specific primers covering 5′ end, middle, and 3′ end of HER-2. The PCR-analysis showed that the topoIIα probe did not recognize sequences from HER-2/neu.

Sequencing the ends of the Exemplary probe revealed that this sequence is bounded on the 3′ end by the sequence shown in FIG. 2A (SEQ ID NO:9), and bounded on the 5′ end by the sequence shown in FIG. 2B (SEQ ID NO:10). Comparison with the published human genome sequence in chromosome 17q 11-21 region in Gene Bank revealed that the sequence of the Exemplary probe is located about 500 kb downstream of the HER2/neu gene.

The sequence of the Exemplary probe may be constructed, for example, by employing the 3′ and/or 5′ ends of the Exemplary probe sequence (i.e. SEQ ID NOS:9 and 10). For example, these sequences may be used to screen a library of human sequences, such that a clone containing this sequence is found and isolated. This clone can be further manipulated by standard molecular biology techniques such that sequences similar to, or identical to, the Exemplary probe sequence are generated. SEQ ID NOs:9 and 10 may also be employed to screen human gene sequence databases (e.g. at chromosome 17) such that the sequences between SEQ ID NOs:9 and 10, and near SEQ ID NOs:9 and 10, may be determined (and then used to generate sequences that are the same or similar to the Exemplary probe sequence using standard molecular biology techniques). Preferably, if sequences similar to the Exemplary probe sequence are generated, the length of the resulting sequence is selected such that it is between 100 kb and 1 megabase (total length of the library of fragments that make up the probe) and is capable of hybridizing to human chromosome 17 (e.g., at a region that contains the TopoIIα gene and not the HER2/neu gene).

To confirm that the Exemplary probe contained the TopoIIα gene sequence, a PCR test was conducted. In particular, the Exemplary probe sequence was used as a template and Two topoIIα primers were used (TopoIIα: 5-′GCC TCC CTA ACC TGA TTG GTTA-3′, SEQ ID NO:11; and TopoIIαB: 5′-CTC AAG AAC CCT GAA AGC GACT-3′, SEQ ID NO:12). The PCR reaction was performed in a volume of 25 ul containing 100 ng of Tracer DNA, 20 pmols of each primer, 1×KlenTaq DNA polymerase (Clonetech), and 200 uM of each dNTPs (Roche). The PCR was performed for 30 cycles of 94 degrees Celsius for 1 minute. The resulting gel revealed a clear TopoIIα PCR product (259 bases). This same type of PCR test may be used on other TopoIIα probe sequences that are generated to confirm that the TopoIIα gene is encompassed by the probe.

ii) Preparation of Tracer DNA

The Exemplary topoIIα probe (or “tracer DNS) may be used to generate a library of fragments with a total length of 100 kb to 1 megabase (e.g. 170 kb total length), with the repetitive sequences substantially removed from this library as described below. In this example, the trace DNA, was prepared by sonication of 30 ug of purified PAC clone DNA to 0.1-8 kb and size-fractionated on 1% agarose gel. The 0.5-4 kb fractions were cut from the gel, purified using QIAquick gel extraction kit (QIAGEN, Santa Clarita, Calif.), blunt ended and ligated to the TopoIIα1/TopoIIα2 adapter. The TopoIIα1/TopoIIα2 adapter was constructed by annealing polyacrylamide gel electrophoresis (PAGE)-purified oligos TopoIIα1 5′-(PO4) GCT ACG GTC TGC TCA GGA CAG TT-3′ (“antisense” oligo, SEQ ID NO:13), and TopoIIα2 3′-CGA TGC CAT ACG AGT CCT GTC-5′ (“sense” oligo, SEQ ID NO:14). Adapter ligated fragments (100 ng) were then PCR amplified, in multiple 25 ml reactions, using the TopoIIα2 sequence as primer. PCR cycling conditions were 94° C. for 30 sec, 60° C. for 30 sec and 72° C. for 3 min for 30 cycles, followed by 72° C. for 10 min. Amplified fragments were size-fractioned (0.5-4 kb fractions) on 1% agarose gel, then purified using QIAquick gel extraction kit.

iii) Preparation of Biotin-Labeled Driver DNA

Fragments of high molecular weight Cot-1 (3 mg) ranging in size from 0.4 and 2 kb were gel purified, blunt ended, and ligated to a D-40/D-41 adapter constructed by annealing PAGE-purified oligos 5′AATTCTTGCGCCTTAAACCAAC (D-40) SEQ ID NO:15 and 5′GTTGGTTTAAGGCGCAAG (D-41) SEQ. ID. NO: 16. Adapter ligated fragments (100 ng) were then PCR amplified, in multiple 25 ml reactions, using 5′end biotin-labeled D40 (5′ (biotin) AATTCTTGCGCCTTAAACCAAC (D-40B) SEQ. ID. NO:17) sequence as primer. PCR cycling conditions were 94° C. for 30 sec, 60° C. for 30 sec and 72° C. for 3 min for 30 cycles, followed by 72° C. for 10 min. The PCR product was purified by phenol:chloroform:isoamyl. The pellet was dried and drive DNA carefully re-dissolved at 1.5-2.5 mg/ml in EE buffer (10 mmol/L 2hydroxyethyl] piperazine-N′-3-propanesulfonic acid (NaEPPS), 1 mmol/L EDTA, pH 8.0).

iv) Subtraction Hybridization

Genomic subtractive hybridization removed sequences from a tracer DNA population by hybridizing with a molar excess of driver DNA. The driver DNA is chemically modified, e.g. with a biotin, such that it may be selectively removed from solution along with driver-tracer hybrid molecules. Briefly, TopoIIα tracer DNA was repeatedly hybridized with 40-fold excess of biotin-labeled Driver DNA containing the repetitive sequences (Alu and LINE elements). Consequently, repetitive sequences present in the TopoIIα region were quantitatively removed. The detailed methods are set forth below.

Subtraction was performed by mixing 250 ng of tracer DNA with 10 mg of biotin-labeled driver DNA, 2 mg of TopoIIα2, 5 mg of yeast tRNA as carrier. This mixture was denatured at 100° C. for 2 min, lyophilized, redissolved in 5 ml of EE buffer/I mol/L NaCl, then incubated at 65° C. for 24 to 48 hours. Biotinylated molecules (including tracer-driver hybrids) were removed using avidin-polystyrene beads as described. Remaining unbiotiylated tracer fragments were precipitated in ethonal before proceeding with next round of subtraction. Each of three rounds of subtraction was performed as described above. The subtraction process resulted in at least 95% of the repetitive sequences being removed from the probe library. After the third round, remaining tracer fragments were amplified by PCR using the TopoIIα2 sequence as primer.

v). Subtracted Nucleic Acid Probe Library Final Preparation

After three rounds of subtraction and 3 rounds of PCR, the TopoIIα subtracted nucleic acid probe library was labeled with digoxigenin (DIG) using the random octamer primer kit (Gibco BRL/Life Technologies). The final nucleotide concentrations for DIG labeling were 0.2 mmol/L dCTP, dGTP, dATP, 0.13 mmol/L dTTP, and 0.07 mmol/L Dig-11-dUTP (Boehringer Mannheim/Roche)/Residual primers and unincorporated nucleotides were removed by S-200HR spin column chromatography (Amersham Pharmacia Biotech Inc.). The purified products were precipitated in ethanol and dissolved in a solution containing 50% formamide, 10% dextran sulfate, and 2×SSC (0.3 mol/L sodium chloride, 0.03 mol/L sodium citrate, pH 7.0).

While the Exemplary probe topoIIα library is labeled with digoxigenin (DIG). The Exemplary probe sequence, or other probes with the same or similar sequences, can be labeled with any type of detectable label (e.g., such that the probe can be detected during in situ hybridization procedures such as FISH or CISH). Also, the Exemplary probe's specificity has been demonstrated by CISH detection methods (data not shown) on the mammary gland adenocarcinoma MCF-7 (ATCC# HTB-22) which does not have TopoIIα gene amplification or deletion, and mammary gland adenocarcinoma cells MDA-MB-361 cell (ATCC# HTB-27), which has the TopoIIα gene deleted.

### Example 11

In Situ Hybridization Methods with the Exemplary TopoIIα Probe

This example describes in situ hybridization methods (CISH and FISH) that may be used with TopoIIα probes, such as the Exemplary TopoIIα Probe described in Example 8. In particular, this Example describes in situ hybridization methods with the Exemplary probe in Formalin-Fixed, Paraffin-Embedded (FFPE) Tissue Samples, as well as Cell Sample/Metaphase Chromosome samples. Finally, this example describes a quality control procedure that may be used with any of these methods.

A. Single-Color CISH For Detection of DIG Labeled Exemplary TopoIIα Probe on FFPE Tissue Sections

I. Pretreatment

| | |
|---|---|
| 1. Deparaffinization | |
| Xylene | 10 Min × 2 |
| 100% EtOH | 5 Min × 3 |
| Air dry slides | |
| 2. Heat treatment | |

(boil the slide by using microwave with temperature probe, or pressure
cooker or hot plate)

| | |
|---|---|
| Tris-EDTA buffer, pH 7.0 | 15 Min, 96-100° C. |

(SPOT-Light Tissue Heat Pretreatment Buffer, Cat.# 00-8401)

| | |
|---|---|
| dH₂O | 2 Min × 3 |
| 3. Pepsin digestion: | |
| Pepsin at 37° C. | 3 Min. |

Note: different concentrations of pepsin and incubation times (1-10 min)
may be required depending on tissue fixation and type of tissue. Excessive
digestion will cause loss of nuclei and chromosome structure, while
inadequate digestion may result in loss of signal.

| | |
|---|---|
| dH₂O | 2 min × 3 |
| 4. Dehydration with graded alcohol | |
| 70% EtOH | 2 min |
| 85% EtOH | 2 min |
| 95% EtOH | 2 min |
| 100% EtOH | 2 min |
| 100% EtOH | 2 min |
| 5. Air dry slides | |
| 6. Label slides with pencil | |

II. Option 1: Co-Denaturation and Hybridization:
(Use PCR Machine with Slide Block, or Heating Block with Temperature Digital Display and Humidity Slide Chamber and 37° C. Incubator)
1. Add probe: add 12-15 ul of probe to the center of 22×22 mm coverslip, or 20 ul of probe to the center of 24×32 mm coverslip.
2. Coverslip: coverslip slide at appropriate tissue sample area.
3. Seal with rubber cement: seal edges of coverslip with thin layer of rubber cement for preventing evaporation during incubation.
4. Denaturation at 94° C. for 5 min: place the slides in a slide block of PCR machine, or on a heating block with temperature digital display.
5. Incubation at 37° C. overnight: leave the slides in the slide block of PCR machine or place the slides in a dark humid box in a incubator.

Option 2: Separate Denaturation (e.g., when PCR Machine or Heating Block are not Available)

| | |
|---|---|
| 1. Denature tissue in fresh made denaturing buffer at | 5 min. |
| 75° C. | |

Denaturing buffer: 4 ml 20 × SSC (20 × SSC buffer = 0.3M
Sodium Citrate, with 3M NaCl, ph 7.0), 8 ml ddH₂O, 28 ml formamide.
(For more than one slide samples, add 1° C. per slide. For example,
if 2 slides are used, set temperature to 76° C.).

| | |
|---|---|
| 2. Dehydration with graded alcohol | |
| 70% EtOH | 2 min, at −20° C. |
| 85% EtOH | 2 min, at −20° C. |
| 95% EtOH | 2 min, at −20° C. |
| 100% EtOH | 2 min, at RT |
| 100% EtOH | 2 min, at RT |
| 3. Air dry slides. At the same time process step 4. | |
| 4. Denature labeled probe | 75° C., 5 min. |
| 5. Place denatured probe in ice immediately. | |
| 6. Add probe: add 12-15 ul of denatured probe to | |
| the center of 22 × 22 mm coverslip. | |
| 7. Coverslip slides at appropriate tissue sample area. | |
| 8. Incubation: place slides in a dark humid box at | |
| 37° C. for overnight (more than 14 hours). | |

III. Stringency Wash:

| | |
|---|---|
| 1. After hybridization, carefully remove rubber cement | |
| and coverslip. | |
| 2. Stringency wash: Wash slides in 0.5 × SSC at | 75° C. for 5 min. |

(Add 1° C. per slide for more than 2 slides, but do not go higher than
80° C.)

| | |
|---|---|
| 3. dH₂O wash: | 2 min × 3 |

IV. Immunodetection:

| | |
|---|---|
| 1. 3% H₂O₂ in absolute Methanol: (for Peroxidase | 10 min |
| Quenching) | |
| 2. 1 × PBS (10 mM)/Tween 20 (0.025%) wash: | 2 min × 3 |
| 3. Add blocking reagent 2 drops/slide at RT | 10 min |
| (CAS-Block; 0.25% casein, 0.2% gelatin, and 10 | |
| mM PBS, pH 7.4) | |

Note: use enough reagents to cover all the area of tissue.

| | |
|---|---|
| 4. Blot off blocking reagent, DO NOT RINSE. | |
| 5. Add FITC-anti-dig antibody 2 drops/slide at RT | 45 (30-60) min |

Note: use enough reagents to cover all the area of tissue.

| | |
|---|---|
| 6. 1 × PBS/Tween 20 (0.025%) wash | 2 min × 3 |

If FISH is desired, add 1 drop of VECTASHIELD Mounting Medium
with DAPI (Vector, Cat. No. H-1200) on the section, then coverslip.
Incubate for 10 min at RT in a dark chamber box before performing
fluorescent microscopy. After analysis is done, remove coverslip, wash
slide in 1 × PBS/Tween 20 (0.025%) 3 times, each time
2 min. Continue to next step.

| | |
|---|---|
| 7. Add HRP-anti-FITC 2drops/slide at RT | 45 (30-60) min |

Note: use enough reagents to cover all the area of tissue.

| | |
|---|---|
| 8. PBS/Tween (0.025%) wash | 2 min × 3 |
| 9. Add DAB, 3 drops/slide, | 30 min |

Note: use enough reagents to cover all the area of tissue.

(Make DAB signal by adding 1 drop of each reagent A (CAS-BLOCK),
B (FITC-Sheep anti-Digoxigenin) and C (HRP-Goat anti-FITC) to 1 ml
dH₂O, then mix well)

| | |
|---|---|
| 10. Wash with running tap water: | 2 min. |

V. Counterstaining and Coverslipping

| | |
|---|---|
| 1. Counterstain with hematoxylin | 6 sec-1 min. |

Time of counterstaining is dependent on tissues used. Dark
counterstaining is not recommended as it may obscure the positive signal.

| | |
|---|---|
| 2. Wash with running tap water | 2 min |
| 3. Dehydrate with graded EtOH | 2 min each |
| (70%, 85%, 95%, 100%, 100%) | |
| 4. Xylene | 2 min × 2 |
| 5. Coverslip with Histomount (Cytoseal 6.0,cat. # 8310-16, | |
| Stephen Scientific). | |

V. Microscopy

Visualize probe in cells with a bright field microscope.

B. Cell Sample or Metaphase Chromosome Sample

Fix cell sample on HISTOGRIP or Superfrost/Plus coated (or other) glass slide.

Pretreatment

1. Immerse slides in 2×SSC buffer (20×SSC buffer=0.3M Sodium Citrate, with 3M NaCl, ph 7.0) at 37 degrees Celsius for 60 minutes.
2. (Optional) Pretreat cells with SPOT LIGHT Cell Pretreatment Reagent (or other Pepsin composition in acidic buffer) for 5 minutes at 37 degrees Celsius. Incubation time may be from 1-10 minutes depending on cell type and slide-making conditions. Excessive pepsin digestion will cause loss of nuclei and chromosome structure. Inadequate digestion may result in loss of signal.
3. Wash in dH₂O for 3×2 minutes at room temperature (RT).
4. (Optional) Immerse slides in 10% buffered formalin for 1 minute at RT.
5. Wash in dH₂O for 3×2 minutes at RT.
6. Dehydrate slides in 70%, 85%, 95%, and 100% ethanol for 2 minutes each, and then air dry.

Slides are now ready for ISH procedure (alternatively, slides can be stored in 70% ethanol at −20 degrees Celsius.

Denaturation and Hybridization

1. Add 15 ul of Exemplary topoIIα probe (probe) to the center of the sample and cover with a 22×22 mm coverslip (use more probe for bigger sample and larger coverslip).
2. Seal edges of coverslip with thin layer of rubber cement to prevent evaporation of probe solution during incubation.
3. Denature the slides on a hot plate or slide warmer at 80 degrees Celsius for 3 minutes (2-5 minute range), or in the slide block of a PCR thermal cycler.
4. Place slide in a dark humidity box or in the slide block of a PCR thermal cycler for 16-24 hours at 37 degrees Celsius.
   Stringency Wash
1. Remover rubber cement and coverslip.
2. Immerse slides in 0.5×SCC buffer, using a Coplin jar, for 5 minutes at 72 degrees Celsius (note—this temperature is based on one slide, but each slide causes a 1 degree Celsius drop in solution temperature. Therefore, if there is more than one slide, adjust the water bath temperature accordingly. For example, if washing 4 slides, adjust the water bath temperature to 75 degrees Celsius. Do not go higher than 80 degrees Celsius.).
3. Wash slides in PBS/Tween 20 buffer (1 part Tween-20, 3900 parts 0.1 M PBS) for 3×2 minutes at RT.

Perform Immunodetection and Counterstaining-Coverslipping as described above in part A above.

C. Quality Control Procedures

Quality control over the accuracy of the above procedures may be assured by using some or all of the controls described below.

Positive (Amplification) Tissue Control: External positive control materials for clinical research should be fresh autopsy/biopsy/surgical specimens fixed, processed, and embedded as soon as possible in the same manner as the patient sample(s). Specimens processed differently from the specimen sample(s) validate reagent performance, and do not verify tissue preparation. Positive tissue controls are indicative of correctly prepared tissues and proper staining techniques. One positive tissue control for each set of test conditions should be included in each run.

Tissues used for the positive control materials should be selected from specimens with well-characterized levels of TopoIIα gene. Approximately 5-10% of breast cancer tissue has TopoIIα gene amplification and may be a useful source of positive control tissue.

Known positive controls should be utilized for monitoring the correct performance of processed tissues and test reagents, rather than as an aid in interpreting sample results. If the positive tissue controls fail to demonstrate positive staining, results with the specimen samples should be considered invalid.

Negative or Normal (Diploid) Tissue Control: Human diploid tissue samples normally have two TopoIIα gene copies in each cell. Therefore, a true negative tissue sample is not available. However, normal tissue can be used as a negative control for gene amplification or deletion. Use a negative tissue control (known to be diploid) fixed, processed, and embedded in the same manner as the sample(s) with each staining run. This will verify the specificity of the ISH probe, and provide an indication of non-specific background staining (false positive staining).

A negative tissue control that is separate from the sample is known as an ‘external’ negative control. If an external negative tissue control is not available then a normal section of the sample can serve as an ‘internal’ negative tissue control.

Reagent (No-Probe) Control: A reagent control is run on a section of sample specimen without the probe. The reagent control is useful in evaluating the possibility of nonspecific staining, particularly when performing ISH in tissue sections. The reagent control should be stained in the same way as the test samples except that hybridization buffer, that does not contain the probe, should be used during the hybridization step. Slide pretreatment, denaturation, and immunodetection should be performed under the same conditions as test samples.

### Example 12

### Predictive Value of TopoIIα and HER-2/neu Amplification in Primary Tumors Cells-CISH TopoIIα Detection

This example describes the predictive value of dual amplification of topoIIα and HER-2/neu in regards to clinical response to topoisomerase II inhibitor chemotherapy. In particular, CISH was used to determine the topoIIα gene copy status for the same primary tumor (breast cancer) patient samples determined to have HER-2/neu amplification as described in Example 6. However, the paraffin block material was exhausted for 16 tumors, so only 45 patient samples were used in this Example, instead of the full 61 tumor samples tested by FISH in Example 6 (See, Table 6).

Slides were de-paraffinized and incubated in 0.1 M Tris-HCl (pH 7.3) in a temperature-controlled microwave oven (at 92 degrees Celsius for 10 minutes, followed by cooling down for 20 minutes at room temperature). After a wash with PBS, enzymatic digestion was done by applying 100 ul of digestion enzyme on to slides for 10-15 min at room temperature (Digest-All III solution, which is a 0.25% pepsin enzyme solution, sold by Zymed Inc., South San Francisco, Calif.). The slides were then washed with PBS and dehydrated with graded ethanols. The ready-to-use digoxigenin-labeled DNA probe for topo IIα (i.e. the Exemplary topoIIα probe described in Example 8, available from Zymed Labs.) was applied onto slides which were covered under 18×18 mm coverslips (10 ul probe mixture/slide). The slides were denatured on a thermal plate (at 94 degrees Celsius for 3 minutes), and the hybridization was carried out overnight at 37 degrees Celsius. After hybridization, the slides were washed with 0.5×SSC (standard saline citrate; 5 min at 75 C), followed by three washes in PBS. The results of this Example are shown in Table 12 below. A comparison of the results using FISH (table 6) and CISH (table 12) to detect topoIIα status is presented in table 13 below.

TABLE 12
Association of TopoIIα Gene Aberrations with Clinical Response To
Chemotherapy in 45 HER-2/neu Positive Breast Cancer Samples

| Response to | | | Not | |
|---|---|---|---|---|
| Chemotherapy | CR or PR | NC or PD | evaluable | Total |
| No amp. | 7 | 16 | 3 | 29 |
| TopoIIα | 12 | 4 | 3 | 19 |
| amplification | | | | |
| Total | 19 | 20 | 6 | 45 |

P-value = 0.0095 (excluding “NE” = response not evaluable);
CR = complete response
PR = partial response;
NC = no change in disease status;
PD = progressing disease.

TABLE 13
Comparison of TopoFish (Table 6) v. TopoCish (Table 12)

topoFISH

| topoCISH | normal-or-del | amp | TOTAL |
|---|---|---|---|
| no amp | 25 | 1 | 26 |
| amp | 4 | 15 | 19 |
| TOTAL | 29 | 16 | 45 |

kappa coefficient k = 0.767 (considered as “excellent agreement”)

### Example 13

### Generation of EGFR Subtracted Probe Library

This example describes the generation of an EGFR subtracted probe library. This probe library was generated substantially as described in Example 7, except where otherwise specified.

1) Selection of the BAC and PAC Clone for Detection of EGFR Gene Amplification.

Two BAC clones (343B1 and 339F13), one PAC clone (1091E12) were identified by human genome project (obtained from Research Genetics). These three clones were confirmed to contain EGFR gene by PCR, and FISH using each clone showed all of them bind specifically to the EGFR gene locus on chromosome band 7p12 with absence of chimerism. A BLAT search of the UCSC genome browser Aug. 6, 2001 freeze indicated that the three clones overlapped, and combined to span a 303 kb distance from 59711021-60014071, that encompassed the EGFR gene.

2). Preparation of Tracer DNA.

The PAC and BAC clones were manipulated in the same manner as described in Example 7 above. The primers for adapter used for the EGFR probe library are as follows:

(SEQ ID NO:20)
EGFR.A 5′-(PO4) ACC GTA GGA CTC TGC TGG CGA TT-3′ (“antisense” oligo),
and
(SEQ ID NO:21)
EGFR.B 5′-TCG CCA GCA GAG TCC TAC GGT-3′ (“sense” oligo)

3). Preparation of Biotin-Labeled Driver DNA

Same as Example 7.

4). Subtraction Hybridization

Same as HER2 probe (Example 7) except T1 primer was replaced by EGFR B primer.

5). Probe Preparation

Same as HER2 probe (See, Example 7).

### Example 14

### ABL Subtracted Split-Apart Probe Pair

This example describes the generation of an ABL split-apart subtracted probe pair library. This probe library was generated substantially as described in Example 7, except where otherwise specified. Importantly, this probe pair is designed to detect chromosome translocations in a unique “split-apart” manner. Conventional BCR/ABL probe pairs are located on different chromosomes in the “normal” state (e.g. non-cancerous), and are only located side by side when translocation occurs. The spit-apart ABL probes of the present example are designed such that that pair is located on the same chromosome in the “normal” state (e.g. non-cancerous), and are only located on separate chromosomes when translocation has occurred in the sample being tested.

1) Selection of the BAC Clones for Detection of BCR/ABL Translocation.

Two BAC clones, RP11-618A20 (accession No. AL354898.10) and RP11-17L7 (accession No. AL353695.7) were used to generate the ABL.c (centromeric side) probe (about 258 kb in size.). FIG. 10 shows the UCSC genome browser for the ABL gene, which may be used to selected additional or alternate clones that may be used to generate the ABL.c and ABL.t probes of the present invention. Also, two BAC clones, RP11-143H2O (accession No. AL355872.13) and RP11-544A12 (accession No. AL157938.22) were used to generate the ABL.t (telomeric side) probe (about 250 kb in size). FISH using these clone showed all of them bind specifically to chromosome band 9q34 with an absence of chimerism.

2). Preparation of Tracer DNA.

The BAC clones were manipulated in the same manner as described in Example 7 above. The primers for adapter:

(SEQ ID NO:22)

ABL.cA 5′-(PO₄) ATC GGT GTA GCC TGA ATG GAG TT-3′

(SEQ ID NO:23)

ABL.cS 5′- GTC CAT TCA GGC TAC ACC GAT-3′

(SEQ ID NO:24)

ABL.tA 5′-(PO₄) CAT CAT TCG GTC AGA GGC ACT TT-3′

(SEQ ID NO:25)

ABL.tS 5′-AGT GCC TCT GAC CGA ATG ATG-3′

3). Preparation of Biotin-Labeled Driver DNA

Same as Example 7.

4). Subtraction Hybridization

Same as HER2 probe (Example 7) except T1 primer was replaced by ABL.cS and ABL.tS primers for ABL.c and ABL.t probe respectively.

5). Probe Preparation

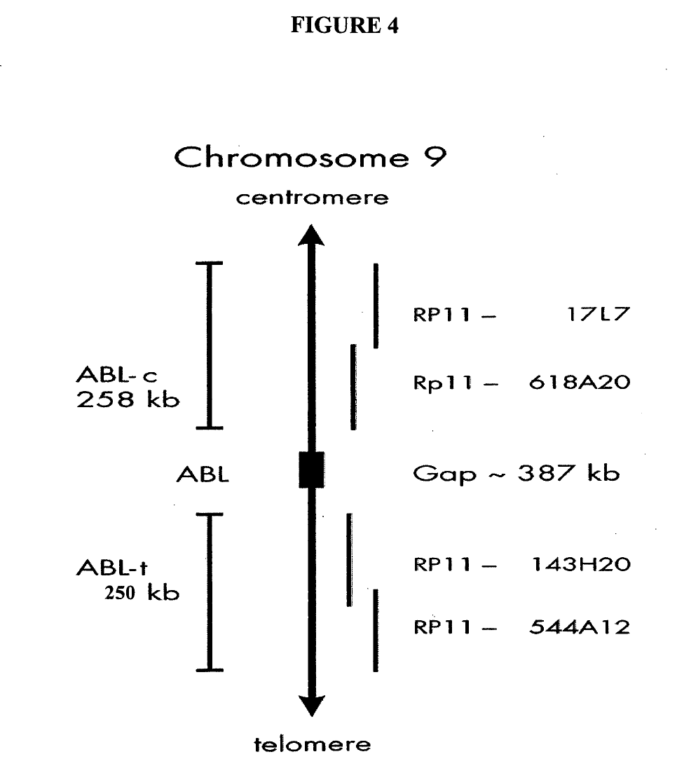

Same as HER2 probe (See, Example 7). The probe pair (centromeric and telemoric probes) give about a 109 kb gap on the centromeric side and a 104 kb gap on the telomeric side of the ABL gene (See FIGS. 4 and 5). This labeled probe pair, for example, may be used to detect ABL translocations (e.g. BCR-ABL translocation) by in situ hybridization techniques (e.g. FISH and CISH). For example, translocations in the ABL gene are found in CML, acute lymphoblastic leukemai (ALL) acute non-lymphocytic leukemia (ANLL), and acute myeloid leukemia (AML) (See, section VI above). In addition these probes can detect variant ABL translocations, such as TEL-ABL, which are found in other types of leukemia.

### Example 15

### Generation of N-MYC Subtracted Probe Library

This example describes the generation of an N-MYC subtracted probe library. This probe library was generated substantially as described in Example 7, except where otherwise specified.

1) Selection of the BAC Clone for Detection of N-Myc Gene Amplification.

Two BAC clones (2014F22 and 2121A13339F13) were identified by Caltech OncoBAC screening effort and human genome project (obtained from Research Genetics). These two clones were confirmed to contain N-Myc gene by PCR. FISH using each clone showed all of them bind specifically to the N-MYC gene locus on chromosome band 2p24.3 with an absence of chimerism. The FISH signal generated using the two clones together was larger than that generated using either clone on its own.

2). Preparation of Tracer DNA.

The BAC clones were manipulated in the same manner as described in Example 7 above. The primers for adapter:

(SEQ ID NO:26)
AF3S 5′-TCT TCA CGA CAC GAC AGC CAG-3′ (“sense” oligo)

(SEQ ID NO:27)

AF3a 3′-TT AGA AGT GCT GTG CTG TCG GTC (PO₄)-5′ (“antisense” oligo)

3). Preparation of Biotin-Labeled Driver DNA

Same as Example 7.

4). Subtraction Hybridization

Same as HER2 probe (Example 7) except T1 primer was replaced by AF3S primer.

5). Probe Preparation

Same as HER2 probe (Example 7).

### Example 16

### Generation of SYT Subtracted Probe Pair Library

This example describes the generation of an SYT (Synovial Sarcoma) subtracted probe pair library. This probe library was generated substantially as described in Example 7, except where otherwise specified. Importantly, this probe pair is designed to detect chromosome translocations in a unique “split-apart” manner. Conventional SYT probe pairs are located on different chromosomes in the “normal” state (e.g. non-cancerous), and are only located side by side when translocation occurs. The spit-apart SYT probes of the present example are designed such that that pair is located on the same chromosome in the “normal” state (e.g. non-cancerous), and are only located on separate chromosomes when translocation has occurred in the sample being tested.

1. Selection of the BAC Clones for Detection of SYT Translocation.

Clones from both sides of the SYT (SS18) gene are employed to generate a split-apart subtracted probe pair library. For example, one or more of the following clones may be used to generate the SYT.c (centromere side probe): RP11-885J19 (See, accession No. AP001326); RP11-689N18 (See accession No. AP001121); RP11-5F22 (See, accession No. AC011268); RP11-326M20 (See accession No. AC019306); RP11-540M4 (See, accession No. AC007768). Preferably the first four clones listed are employed, and optionally the fifth clone is added. In order to generate the SYT.t (telomeric side probe) one or both of the following clones may be employed; RP11-802C10 (accession No. AP002752), and RP11-774F2 (accession No. AP001451).

2. Preparation of Tracer DNA.

The clones were manipulated in the same manner as described in Example 7 above. The primers for adapter:

(SEQ ID NO:28)

SYT.c1 5′-ATG CGT CCA CCT TGA CCTTAC-3′

(SEQ ID NO:29)

SYT.c2 5′-(PO₄) GTA AGG-TCA AGG TGG ACG CAT-TT-3′

(SEQ ID NO:30)

SYT.tS 5′-ATA GCC CCG AAT CAG GTG GAA-3′

(SEQ ID NO:31)

SYT.tA 5′-(PO₄)-TTC CAC CTG ATT CGG GGC TAT TT-3′

3. Preparation of Biotin-Labeled Driver DNA

Same as Example 7.

4. Subtraction Hybridization

Same as HER2 probe (Example 7) except T1 primer was replaced by SYT.c1 and SYT.tS primers for SYT.c and SYT.t probe respectively.

5. Probe Preparation

Same as HER2 probe (Example 7).

### Example 17

### Generation and Testing of MALT1 Probe Pair

This Example describes the generation of centromeric and teleomeric subtracted probe pair libraries for detecting MALT1 translocation, as well as the use of these probe pair libraries in FISH and CISH methods.

The MALT1 probe pair is composed a centromeric subtracted probe library (MALT.c) and a teleomeric subtracted probe library (MALT.t). These two subtracted probe libraries were generated as follows. Three BAC clones for each probe library were identified using the UCSC human genome browser. The web address for the UCSC human genome browser is genome.ucsc.edu, with the freeze date for this Example, and the Examples below that employ BACs is April of 2003. The accession numbers listed in this Example, and the Examples below, can be retrieved by entering the complete clone name on the UCSC Human Genome Browser and clicking on the clone name or by the NCBI Clone Registry at ncbi.nlm.nih.gov/projects/genome/clone/.

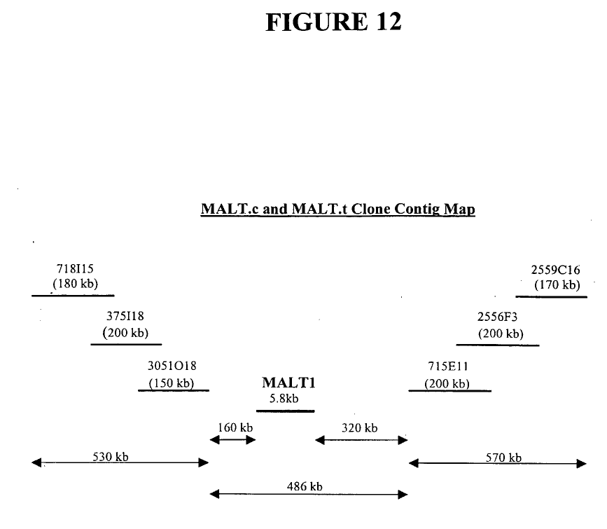

As shown in FIG. 12, BAC clones RP11-718I15 (AC107896), RP11-37518 (BAC end alignments: AZ515667, AZ515668), and CTD-3051O18 (BAC end alignments: AQ734929, AQ735055) were used for MALT.c, and BAC clones CTD-2559C16 (BAC end alignments: AQ427033, AQ427034), CTD-2556F3 (BAC end alignments: AQ393497, AQ425711), and RP11-715E11 (BAC end alignments: AQ517653, AQ513015) were used for MALT.t. The sequences of all six clones are shown in Table 15.

As shown in this diagram in FIG. 12, the centromeric and teleomeric probe libraries are located at considerable distance from the MALT1 gene such that all known break points are able to be detected.

Both probe libraries were subjected to subtraction procedures (to remove repetitive sequences) as described in previous Examples (see, e.g., Example 7). After the tracer DNA is broken down into 0.5 to 4 kb fragments, the adapters added to the fragments (for PCR) are as follows: MALT.c used SPT2A 5′(PO4)-CTG GCT ATC GGC TAT TCG TGC TT-3′ (SEQ ID NO:32), and SPT2S 5′-GCA CGA ATA GCC GAT AGC CAG-3′ (SEQ ID NO:33), while MALT.t used MALT.TA 5′(PO4)-GTC AAG CGT CAT ACT GGC GTT TT-3′ (SEQ ID NO:34) and MALT.TS 5′-AAC GCC AGT ATG ACG CTT GAC-3′ (SEQ ID NO:35). Once 3 rounds of subtraction and 3 rounds of PCR were completed the DNA was labeled using random primed labeling which produces about 0.2-0.5 kb fragments. This procedure resulted in a MALT.c subtracted probe library that is about 530 kb in length when hybridized to the target and a MALT.t subtracted probe library about 570 kb in length when hybridized to the target.

The two subtracted probe libraries were then assayed using FISH methods to confirm their specificity. The two subtracted probe libraries, labeled with digoxigenin, were used in FISH procedures on normal lymphocyte metaphase preparations along with a chromosome 18 centromere control (available from Zymed Labs). The results of the FISH procedure showed that the two subtracted probe libraries bound to chromosome 18 with no translocation. The FISH results also revealed an absence of chimerism (i.e. both MALT.c and MALT.t only bound to their intended target regions, not to other non-intended regions).

The specificity of the MALT1 subtracted probe libraries was also confirmed by BAC end sequencing. Two of the MALT.c clones (375I18 and 3051O18) and two of the MALT.t clones (2556F3 and 715E11) were BAC end sequenced using Sp6 and T7 primers. The sequences were blasted using the UCSC Blat Program (//genome.ucsc.edu) showing specific hybridization to the 5′ and 3′ ends of the BAC clones. MALT.c clone 718I15 and MALT.t clone 2559C16 were not BAC end sequenced but were combined with the other two clones for the centromeric and telomeric MALT clones showing specific hybridization without chimerism Next, the MALT.c and MALT.t subtracted probe libraries were used in various CISH procedures. CISH was performed generally as described in previous Examples, except for modifications such as: i) using a enzyme digestion solution composed of 0.075% pepsin at a pH of 2.0, ii) using an enzyme digestion temperature of room temperature for 10 minutes; and iii) using a stringency wash temperature of at least 75° C., but not exceeding 80° C. When a normal (non-MALT lymphoma) cell line was assayed, the MALT.c and MALT.t probes (represented by red and brown dots) were found together in the cells, correctly indicating no translocation. When a MALT lymphoma tissue was assayed, the MALT.c and MALT.t probes were separated (the red and brown dots were separated) confirming that this probe pair can correctly detect MALT translocations.

### Example 18

### Generation and Testing of Chromsome 19q Deletion Probe

This Example describes the generation of a subtracted probe library for detecting a deletion in chromosome 19q, as well as the use of this subtracted probe library in FISH and CISH methods.

The chromosome 19q subtracted probe library was generated as follows. One BAC clone, CTD-2571L23 (accession no. AC008745.7; see Table 15), was selected. This BAC clone was identified using the UCSC human genome browser. This BAC clone is approximately 181 kb in size and spans region 19q13.33.

The tracer DNA from this one BAC clone, after being broken down into 0.5 to 4.0 kb fragments, was subject to subtraction procedures (to remove repetitive sequences) as described in previous Examples (see, e.g., Example 7). The following adapters are added to the tracer DNA fragments for PCR: 19p19q.S 5′-GCA GCA TTA CGC CTT TCC GAG-3′ (SEQ ID NO:36) and 19p19q.A 5′-(PO4)CTC GGA AAG GCG TAA TGC TGC TT-3′ (SEQ ID NO:37). Once 3 rounds of subtraction and 3 rounds of PCR were completed the DNA was labeled using random primed labeling which produces about 0.2-0.5 kb fragments. This procedure resulted in a chromosome 19q subtracted probe library that is about 181 kb in length when hybridized to the target sequence.

The 19q subtracted probe library was then assayed using FISH methods to confirm its specificity. The 19q subtracted probe library, labeled with digoxigenin, was used in a FISH procedure on normal lymphocyte metaphase preparations along with a chromosome 19 control labeled with biotin. The results of the FISH procedure showed that the subtracted probe library bound to chromosome 19. The FISH results also revealed an absence of chimerism (i.e. the probe 19q subtracted probe library only bound to its intended target regions, not to other non-intended regions).

The specificity of the chromosome 19q subtracted probe library was also confirmed by PCR. Two chromosome 19q primers were employed, 19QF: 5′-GCT ACA GTG ACA GAT TTT AGA GCA-3′ (SEQ ID NO:38) and 19QR: 5′-TGA AAG TTT TAG AAG CCT CCA T-3′ (SEQ ID NO:39) were employed for PCR. The PCR reaction was performed in a volume of 25 μl containing 100 ng of Tracer DNA, 0.1 uM of each primer, 1×KlenTaq DNA polymerase (Clontech), and 200 μM of each dNTP (Roche). PCR was performed for a total of 30 cycles at 94° C. for 30 sec., 60° C. for 1 min., and 72° C. for 1 min. These primers were found to amplify the expected 219 PCR product, demonstrating that the 19q probe contains the chromosome 19q13.3 sequence.

Next, the 19q subtracted probe library was used in various CISH procedures. CISH was performed as described in previous Examples, except for modifications such as: i) using an enzyme digestion solution composed of 0.075% pepsin at a pH of 2.0, ii) using an enzyme digestion temperature of room temperature for 10 minutes; and iii) using a stringency wash temperature of at least 75 C, but not exceeding 80 C. When a normal breast tissue sample was assayed, the digoxigenin labeled 19q probe showed two dots as expected for normal 19q gene copy number, correctly indicating no deletion. When a oligodendroglioma tissue sample was assayed by CISH, only one dot per cell was observed, which correctly reveals the deletion of 19q in these cells.

### Example 19

### Generation and Testing of Chromosome 18q Deletion Probe

This Example describes the generation of a subtracted probe library for detecting a deletion in chromosome 18q, as well as the use of this subtracted probe library in FISH and CISH methods.

The chromosome 18q subtracted probe library was generated as follows. Three BAC clones (RP11-718I15, RP11-375I18, and CTD-3051O18), which are the same clones as used for the MALT.c probe (see Example 17) were used to generate chromosome 18q probe. These BAC clones were identified using the UCSC human genome browser.

The tracer DNA from BAC clones, after being broken down into 0.5 to 4.0 kb fragments, was subject to subtraction procedures (to remove repetitive sequences) as described in previous Examples (see, e.g., Example 7). The following adapters are added to the tracer DNA fragments for PCR: SPT2A 5′(PO4)-CTG GCT ATC GGC TAT TCG TGC TT-3′ (SEQ ID NO:40), and SPT2S 5′-GCA CGA ATA GCC GAT AGC CAG-3′ (SEQ ID NO:41). Once 3 rounds of subtraction and 3 rounds of PCR were completed the DNA was labeled using random primed labeling which produces about 0.2-0.5 kb fragments. This procedure resulted in a probe size of about 530 kb when hybridized to the target sequence.

As described in Example 17, the 18q subtracted probe library (same as MALT.c) was assayed using FISH methods, which confirmed its specificity and demonstrate an absence of chimerism.

The 18q subtracted probe library was used in various CISH procedures. CISH was performed as described in previous Examples, except for modifications such as: i) using an enzyme digestion solution composed of 0.075% pepsin at a pH of 2.0, ii) using an enzyme digestion temperature of room temperature for 10 minutes; and iii) using a stringency wash temperature of at least 75 C, but not exceeding 80 C. When a normal breast tissue sample was assayed, the digoxigenin labeled 18q probe showed two dots as expected for normal 18q gene copy number, correctly indicating no deletion. When a colon carcinoma tissue sample was assayed by CISH, only one dot per cell was observed, which correctly reveals the deletion of 18q in these cells.

### Example 20

### Generation and Testing of Chromosome 1p Deletion Probe

This Example describes the generation of a subtracted probe library for detecting a deletion in chromosome 1p, as well as the use of this subtracted probe library in FISH methods.

The chromosome 1p subtracted probe library was generated as follows. Two BAC clones, CTD-2307E2 (BAC end alignments: AQ014983, AQ014988) and CTD-2504F18 (BAC end alignments: AQ262709, AQ262715) were selected to generate the actual subtracted probe library. These sequences are shown in Table 15. Two other BACs were identified as useful, CTD-02270N9 (BAC end alignments: AQ085830, AQ164023) and CTD-3071L13, but were not used to generate the subtracted probe library in this Example. All of these BAC clones were identified using the UCSC human genome browser.

The tracer DNA from the two BAC clones, after being broken down into 0.5 to 4.0 kb fragments, was subject to subtraction procedures (to remove repetitive sequences) as described in previous Examples (see, e.g., Example 7). The following adapters are added to the tracer DNA fragments for PCR: SPT3A 5′(PO4)-GTC GCT ATC GGC TAA TCG TGC TT-3′ (SEQ ID NO:42) and SPT3S 5′-GCA CGA TTA GCC GAT AGC GAC-3′ (SEQ ID NO:43). Once 3 rounds of subtraction and 3 rounds of PCR were completed the DNA was labeled using random primed labeling which produces about 0.2-0.5 kb fragments.

The 1p subtracted probe library was then assayed using FISH methods to confirm its specificity. The 1p subtracted probe library, labeled with digoxigenin, was used in a FISH procedure on normal lymphocyte metaphase preparations along with a chromosome 1 control. The results of the FISH procedure showed that the subtracted probe library bound to chromosome 1. The FISH results also revealed an absence of chimerism (i.e. the probe 1p subtracted probe library only bound to its intended target regions, not to other non-intended regions).

The specificity of the two probes in the 1p subtracted probe library (CTD-2307E2 and CTD-2504F18) as well a third BAC (CTD-2270N9) was also confirmed by BAC end sequencing using Sp6 and T7 primers. The sequences were blasted using the UCSC Blat Program (//genome.ucsc.edu) showing specific hybridization to the 5′ and 3′ ends of the BAC clones.

### Example 21

### Generation and Testing of NF2 (Chromosome 22q) Deletion Probe

This Example describes the generation of a subtracted probe library for detecting a deletion of the NF2 gene or chromosome 22q (long arm), as well as the use of this subtracted probe library in FISH and CISH methods.

The NF2 subtracted probe library was generated as follows. Three BAC clones 256D12 (173 kb; accession no. AC005529.7), CTD-2021M22 (92 kb; BAC end alignments: B65398, AQ229542), and CTD-3170A23 (122 kb; BAC end alignments: AQ149574, AQ179393) were used to generate the NF2 (Chr.22q) subtracted probe library of 300 kb in size, which covers the entire NF2 gene. These BAC clones were identified using the UCSC human genome browser. The sequences of these three clones are shown in Table 15.

The tracer DNA from these BAC clones, after being broken down into 0.5 to 4.0 kb fragments, was subject to subtraction procedures (to remove repetitive sequences) as described in previous Examples (see, e.g., Example 7). The following adapters are added to the tracer DNA fragments for PCR: AF2A 5′ (PO4)-ACG GTC TTG TAC CCG TGC TTG TT-3′ (SEQ ID NO:44), and AF2S 5′-CAA GCA CGG GTA CAA GAC CGT-3′ (SEQ ID NO:45). Once 3 rounds of subtraction and 3 rounds of PCR were completed the DNA was labeled using random primed labeling which produces about 0.2-0.5 kb fragments. This procedure resulted in a NF2 subtracted probe library that is about 300 kb in length when hybridized to the target sequence.

The specificity of the 3 BAC clones used to generate the NF2 subtracted probe library was confirmed by BAC end sequencing. The three clones (256D12, 3170A23, 2021M22) were BAC end sequenced using Sp6 and T7 primers. The sequences were blasted using the UCSC Blat Program (genome.ucsc.edu), which showed specific hybridization to the 5′ and 3′ ends of the BAC clones.

The NF2 subtracted probe library was then assayed using FISH methods to confirm its specificity. FISH was performed on normal lymphocyte preparation. FISH showed specific hybridization to chromosome 22 using: A) DIG-labeled, pre-subtraction 2021M22 (green signals) and biotin-labeled, pre-subtraction 3170A23 (red signals), and B) DIG-labeled, post-subtraction (three clones combined) probe. The FISH results also revealed an absence of chimerism (i.e. the NF2 subtracted probe library only bound to its intended target regions, not to other non-intended regions).

Next, the NF2 subtracted probe library was used in various CISH procedures. CISH was performed as described in previous Examples, except for modifications such as: i) using an enzyme digestion solution composed of 0.075% pepsin at a pH of 2.0, ii) using an enzyme digestion temperature of room temperature for 10 minutes; and iii) using a stringency wash temperature of at least 75 C, but not exceeding 80 C. When a normal tissue sample known to have no deletion in meningioma was assayed, the digoxigenin labeled NF2 (chromosome 22q) subtracted probe library showed two dots per nucleus as expected, correctly indicating no deletion. When schwannoma samples were assayed by CISH, only one dot per nucleus was observed, which correctly revealed the deletion of the NF2 gene.

### Example 22

### CISH and IHC HER2 Testing in Gastric Carcinoma Samples

This example describes testing gastric carcinoma samples by both CISH and immunohistochemistry (IHC) methods. The frequency of HER2 gene amplification in gastric carcinoma and its correlation with protein overexpression was investigated in 97 formalin-fixed, paraffin-embedded (FFPE) gastric cancers by CISH (see CISH HER2 examples in previous Examples) and IHC (TAB250 antibody was used with standard IHC protocols). In order to grade CISH samples, the following guidelines were used:

| | |
|---|---|
| Amplification | High amplification: >10 dots, or large clusters, or mixture of multiple dots |
| | and large clusters of the HER2 gene present per nucleus in >50% cancer |
| | cells. |
| | A large cluster is an irregularly shaped group of signals, which is greater |
| | than 5 times the diameter of one single dot. A single dot from the tumor or |
| | normal epithelial cells from the same slide should be used as a reference. |
| | Low amplification: >5 dots to 10 dots, or small clusters, or mixture of |
| | multiple dots and small clusters of the HER2 gene present per nucleus in |
| | >50% cancer cells. |
| | A small cluster is an irregularly shaped group of signals, which is 3 to 5 |
| | times the diameter of one single dot. A single dot from the tumor or normal |
| | epithelial cells from the same slide should be used as a reference. |
| No | Polysomy: 3-5 dots of the HER2 gene present per nucleus in >50% of |
| Amplification | cancer cells. |
| | A single dot has a smooth, rounded edge in normal or tumor cells. |
| | Diploid: 1-2 dots of the HER2 gene present per nucleus in >50% of cancer |
| | cells. |
| | A single dot has a smooth, rounded edge in normal or tumor cells. |

Among 97 samples, 58 were from a gastric cancer tissue array (Zymed). CISH was successful in 94 cases (97%). HER2 gene amplification was detected in 14 cases (15%), which is similar as reported before (see, e.g, Tanner et al., Ann Oncol. 2005 February; 16(2):273-8). Among them, 4 cases showed HER2 gene heterogeneity (amplification and not amplification). HER2 protein overexpression (2+ and 3+) was detected in 11 cases. All of them had HER2 gene amplification (Table 13). These results indicate that HER2 gene amplification and concomitant protein overexpression occurs in 15% of gastric cancers, and it often occurs in well and moderate differentiated papillar (26%) and tubular types (21%) (See Table 14, which presents the HER2 CISH results in different histology grades). HER2 gene heterogeneity is a common phenomenon (29%) in gastric cancers with HER2 gene amplification. This Examples indicates that CISH methods (e.g. with light microscope detection) could be used to determine if patients should receive anti-HER2 treatment, such as HERCEPTIN. Such testing is likely to be particularly important in Asia as the incidence of gastric carcinoma is higher in Asia than other parts of world.

TABLE 13
HER2 CISH and IHC results

HER2 CISH

| | | Amplified | Not Amplified |
|---|---|---|---|
| IHC | Positive (2+, 3+) | 11 | 0 |
| | Negative (0, 1+) | 3 | 77 |

TABLE 14
HER2 CISH results in different histology grade (WHO)

| | Histology type | | | | | | | | | |
|---|---|---|---|---|---|---|---|---|---|---|
| | Papillar | | | Tubular | | | | | | |
| | 30 | | | 47 | | | | Signet | | |
| | Well | Moderate | Poor | Well | Moderate | Poor | Mucinous | ring | Undifferentiated | Total |
| Differentiation | 14 | 9 | 7 | 19 | 10 | 18 | 4 | 7 | 6 | 94 |
| HER2 | 4 | 2 | 1 | 5 | 1 | 0 | 0 | 1 | 0 | 14 |
| Amplified | | | | | | | | | | |
| % | 29% | 22% | 14% | 26% | 10% | 0% | | | | |

| | | | | | | |
|---|---|---|---|---|---|---|
| % | 23% | 13% | 0% | 14% | 0% | 15% |

### Example 23

### Dual Color TopoIIα and Chromosome 17 CISH Detection

Detecting both TopoIIα and chromosome 17 centromere on the same slides using dual color CISH procedure could be performed as follows. One could perform tissue pretreatment as described in previous examples. After denaturation, hybridization, stringency wash, blocking of endogenous peroxidase, and Cas-Block™, a cocktail of a DIG-labeled TopoIIα probes (e.g. as described in Example 10) and biotin-labeled chromosome 17 centrome probes (available from Zymed Laboratories) could be detected by sequential incubation of mouse anti-DIG antibody for 30 minutes, a mixture of polymerized HRP conjugated goat anti-mouse and AP-streptavidin for 30 minutes (Zymed), DAB chromogen development for 30 minutes, and Fast Red chromogen development 3 times for 10 minutes each. Tissue sections could be counterstained with hematoxylin, covered with Clear Mount (Zymed), incubated at 37° C. for 2 hours, and then coverslipped. The results of this dual color CISH procedure could be visualized using a 40×, 60×, and 100× oil immersion lenses.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, immunology, chemistry, and molecular biology or related fields are intended to be within the scope of the following claims.

We claim:

1. A method for performing chromogenic in-situ hybridization, comprising;
a) exposing a biological sample to a enzyme digestion solution at about room temperature, wherein said enzyme digestion solution comprises 0.050%-0.1% pepsin,
b) contacting said biological sample with a labeled subtracted probe library configured for detecting a target region, under conditions such that said labeled subtracted probe library hybridizes to said target region in said biological sample,
c) adding a detection molecule linked to an enzyme to said biological sample under conditions such that said detection molecule binds; i) to said labeled subtracted probe library, or ii) an intermediate molecule linked to said subtracted probe library, and
d) adding a calorimetric substrate to said biological sample.

2. The method of claim 1, further comprising a step prior to step a) of preheating said biological sample in a pretreatment buffer at a temperature of at least 98 degrees Celsius.

3. The method of claim 1, wherein said enzyme digestion solution comprises between 0.060% pepsin and 0.80% pepsin, and the method further comprises step f) detecting said target region.

4. The method of claim 1, wherein said contacting comprises a stringency wash step, and wherein said stringency wash step is performed at a temperature of at least 75° C., but not exceeding 80° C.

5. The method of claim 1, wherein said exposing step is conducted for about 10 minutes.

6. The method of claim 1, wherein said detection molecule linked to an enzyme comprises an antibody conjugated to a polymer, wherein a plurality of enzymes are linked to said polymer.

7. A method for performing chromogenic in-situ hybridization, comprising;
a) preheating a biological sample in a pretreatment buffer at a temperature of at least 98 degrees Celsius,
b) exposing said biological sample to a enzyme digestion solution,
c) contacting said biological sample with a labeled subtracted probe library configured for detecting a target region under conditions such that said labeled subtracted probe library hybridizes to said target region in said biological sample,
d) adding a detection molecule linked to an enzyme to said biological sample under conditions such that said detection molecule binds; i) to said labeled subtracted probe library, or ii) an intermediate molecule linked to said subtracted probe library, and
e) adding a calorimetric substrate to said biological sample.

8. The method of claim 7, further comprising step f) detecting said target region.

9. The method of claim 7, wherein said target region is selected from the group consisting of: chromosome 19q, chromosome 18q, chromosome 22q, and chromosome 1p.

10. The method of claim 9, further comprising step f) detecting the presence of chromosome 19q deletion, chromosome 18q deletion, chromosome 22q, or chromosome 1p deletion in said biological sample.

11. The method of claim 9, further comprising step f) detecting the presence of normal diploid expression of chromosome 19q, chromosome 18q, chromosome 22q, or chromosome 1p in said biological sample.

12. The method of claim 7, wherein said detecting comprising visualizing said calorimetric substrate with a bright-field microscope.

13. The method of claim 7, wherein said temperature is from 98 degrees Celsius to 100 degrees Celsius.

14. A method for performing chromogenic in-situ hybridization, comprising;
a) preheating a biological sample in a pretreatment buffer at a temperature of at least 98 degrees Celsius,
b) exposing said biological sample to a enzyme digestion solution,
c) contacting said biological sample with a labeled subtracted probe pair library comprising: i) a first subtracted probe library configured to hybridize to a first region of chromosome 18 that is centromeric with respect to the MALT gene; and ii) a second subtracted probe library configured to hybridize to a second region of chromosome 18 that is teleomeric with respect to said MALT gene, under conditions such that said labeled subtracted probe pair library hybridizes to said first and second target regions in said biological sample,
d) adding a detection molecule linked to an enzyme to said biological sample under conditions such that said detection molecule binds; i) to said labeled subtracted pair probe library, or ii) an intermediate molecule linked to said subtracted probe pair library, and
e) adding a colorimetric substrate to said biological sample.

15. The method of claim 14, further comprising step f) detecting said first and second target regions.

16. The method claim 14, further comprising step f) detecting the presence of said first and second subtracted probe libraries in close proximity within the cells in said biological sample, thereby indicating no translocation.

17. The method claim 14, further comprising step f) detecting the presence of said first and second subtracted probe libraries spaced apart within the cells in said biological sample, thereby indicating translocation.

18. The method of claim 15, wherein said detecting comprising visualizing said calorimetric substrate with a microscope.

19. The method of claim 18, wherein said microscope is a bright-field microscope.

20. The method of claim 18, wherein said visualizing is performed with said microscope at a magnification of 35× to 45×.
